(19) United States
(12) Patent Application Publication
(10) Pub. No.: US 2013/0171240 A1
(43) Pub. Date: Jul. 4, 2013

(54) DRUG CARRIER AND DRUG CARRIER KIT FOR INHIBITING FIBROSIS

(71) Applicant: Nitto Denko Corporation, Osaka (JP)

(72) Inventors: Yoshiro Niitsu, Sapporo-shi Hokkaido (JP); Junji Kato, Sapporo-shi Hokkaido (JP); Yasushi Sato, Sapporo-shi (JP); Joseph E Payne, Oceanside, CA (US); John A Gaudette, Poway, CA (US); Zheng Hou, San Diego, CA (US); Victor Knopov, Oceanside, CA (US); Richard P Witte, San Diego, CA (US); Mohammad Ahmadian, Carlsbad, CA (US); Loren A Perelman, San Diego, CA (US); Yasunobu Tanaka, Osaka (JP); Violetta Akopian, Oceanside, CA (US)

(73) Assignee: NITTO DENKO CORPORATION, Osaka (JP)

(21) Appl. No.: 13/786,883

(22) Filed: Mar. 6, 2013

Related U.S. Application Data
(63) Continuation-in-part of application No. 13/439,330, filed on Apr. 4, 2012.
Application No. 13/439,330 is a continuation of application No. 11/793,736, filed on Apr. 8, 2008, now Pat. No. 8,173,170.
Application No. 11/793,736 is a continuation-in-part of application No. 13/492,424, filed on Jun. 8, 2012.
(60) Provisional application No. 61/494,840, filed on Jun. 8, 2011.

(30) Foreign Application Priority Data
Dec. 22, 2004 (JP) 2004-382791

(51) Int. Cl. A61K 47/18; A61K 31/713; A61K 47/24; A61K 9/127

(52) U.S. Cl. CPC A61K 47/183; A61K 9/127; A61K 31/713; A61K 47/24; A61K 47/18
USPC 424/450; 514/772; 514/785; 514/788; 514/44 A; 514/44 R

(57) ABSTRACT

Disclosed is a stellate cell-specific drug carrier comprising a stellate cell-specific amount of a retinoid derivative and/or a vitamin A analogue, and a drug carrier component other than the retinoid derivative and/or a vitamin A analogue. Also disclosed in a medicine comprising the stellate cell-specific drug carrier, and a drug in an amount effective for controlling the activity or growth of stellate cells.

### CROSS-REFERENCE TO RELATED APPLICATIONS

This application is a continuation-in-part of U.S. Ser. No. 13/439,330, filed Apr. 4, 2012, which is a continuation of U.S. Ser. No. 11/793,736, filed Apr. 8, 2008, now U.S. Pat. No. 8,173,170, issued May 8, 2012, which is a national stage filing under 35 U.S.C. §371 of international application PCT/JP2005/023619, filed Dec. 22, 2005. This application is also a continuation-in-part of U.S. Ser. No. 13/492,424, filed Jun. 8, 2012, which claims the benefit of U.S. Provisional Application No. 61/494,840 filed Jun. 8, 2011. The disclosures of all of the above are hereby incorporated by reference in their entireties for all purposes.

### SEQUENCE LISTING

The present application is being filed along with a Sequence Listing in electronic format. The Sequence Listing is provided as a file entitled KUZU1_{—}010P2_SEQ, created on Mar. 5, 2013, which is 5 KB in size. The information in the electronic format of the Sequence Listing is incorporated herein by reference in its entirety.

### BACKGROUND OF THE INVENTION

1. Field of the Invention

The present invention relates to a drug carrier used in a drug delivery system (DDS) for stellate cells, a medicine containing same, and a kit for preparing said medicine and, in particular, to a medicine and a kit for preparing same wherein an active ingredient is a drug for controlling the activity or growth of stellate cells, and especially a drug targeted at an extracellular matrix constituent molecule secreted by stellate cells, or at one or more molecules having the function of producing or secreting an extracellular matrix constituent molecule. The present invention is further directed to the use of fat-soluble vitamin compounds to target and enhance activity of therapeutic molecules, including siRNA.

2. Description of the Related Art

Fibrosis of the liver is caused by, though not limited to, hepatic stellate cells (HSC) being activated as a result of, for example, viral hepatic disease due to hepatitis B or C virus, nonalcoholic steatohepatitis, malnutrition-related diabetes, parasites, infectious diseases such as tuberculosis or syphilis, intrahepatic congestion due to heart disease, or wound healing of tissue injury, etc. inside the liver accompanying a disorder in the passage of bile, etc., and the excessively produced and secreted extracellular matrix (ECM) such as a plurality of types of collagen molecules and fibronectin being deposited on interstitial tissue. The final stage of hepatic fibrosis is hepatic cirrhosis, and since hepatic failure, hepatocellular carcinoma, etc. are caused, in order to prevent them and/or inhibit the progress thereof, there is a desire for the development of a drug carrier and drug carrier kit for inhibiting at least hepatic fibrosis.

Furthermore, in the pancreas, chronic pancreatitis develops as a result of pancreatic fibrosis by the same mechanism as that for hepatic fibrosis (Madro A et al., Med Sci Monit. 2004 July; 10(7): RA166-70; Jaster R, Mol Cancer. 2004 October 6; 3(1): 26.). However, effective means for inhibiting the progress of pancreatic fibrosis or chronic pancreatitis has not yet been found.

As effective means for inhibiting fibrosis of the liver or the pancreas, there is a possibility that stellate cells are one of the important target candidates (Fallowfield J A, Iredale J P, Expert Opin Ther Targets. 2004 October; 8(5): 423-35; Pinzani M, Rombouts K. Dig Liver Dis. 2004 April; 36(4): 231-42.). In the process of fibrosis, stellate cells are activated by cytokine from Kupffer cells or infiltrating cells and transformed into activated cells, and there is marked production of extracellular matrix (ECM). Stellate cells are known as storage cells for vitamin A, and belong to the myofibroblast family. On the other hand, stellate cells produce matrix metalloproteinase (MMP), its inhibitory factor (TIMP), a cytokine such as TGF-β or PDGF, and a growth factor such as HGF, and play a main role in hepatic fibrosis. Activated stellate cells increase contractile ability and are involved in the regulation of blood flow and, furthermore, they increase the expression of various types of cytokine receptors and become highly sensitive to cytokine.

With regard to therapeutic methods for fibrosis that have been attempted up to the present date, the control of collagen metabolism, promotion of the collagen degradation system, inhibition of activation of stellate cells, etc. can be cited. They include inhibition of TGFβ (known as a factor for activating stellate cells and promoting the production of extracellular matrix (ECM)) using a truncated TGFβ type II receptor (Qi Z et al., Proc Natl Acad Sci USA. 1999 March 2; 96(5): 2345-9), a soluble TGFβ type II receptor (George J et al., Proc Natl Acad Sci USA. 1999 October 26; 96(22): 12719-24), HGF (published Japanese translation 5-503076 of a PCT application; Ueki K et al., Nat Med. 1999 February; 5(2): 226-30), etc., promotion of the production of matrix metalloproteinase (MMP) by means of HGF or an MMP gene-containing vector (Iimuro Y et al., Gastroenterology 2003; 124: 445-458.), inhibition of TIMP, which is an MMP inhibitor, by means of antisense RNA, etc. (Liu W B et al., World J Gastroenterol. 2003 February; 9(2): 316-9), control of the activation of stellate cells by means of a PPARγ ligand (Marra F et al., Gastroenterology. 2000 August; 119(2): 466-78) or an angiotensin-II type I receptor antagonist (Yoshiji H et al., Hepatology. 2001 October; 34 (4 Pt 1): 745-50), inhibition of the growth of stellate cells via inhibition of PDGF action by means of PDGF tyrosine kinase inhibitor, etc. (Liu X J et al., World J Gastroenterol. 2002 August; 8(4): 739-45) and inhibition of the sodium channel by means of amiloride (Benedetti A et al., Gastroenterology. 2001 February; 120(2): 545-56), etc., and apoptotic induction of stellate cells by means of Compound 861 (Wang L, et al., World J Gastroenterol 2004 October 1; 10(19): 2831-2835), gliotoxin (Orr J G et al., Hepatology. 2004 July; 40(1): 232-42), etc. However, in all cases, since the specificity of action and/or the organ specificity are low, there are problems with the effects and with side effects.

With regard to collagen protein synthesis, there are many unclear points with respect to the metabolic route, and a therapeutic method using a drug that inhibits this has not been established as a therapeutic method that is efficient and safe toward a living body in terms of side effects. That is, in a method in which molecules involved in the production of collagen are targeted, the specificity for the target cannot be enhanced because of the diversity of function of the molecules, and the possibility of causing side effects is high. If collagen, which is the final product, could be inhibited directly, this would be reasonable as a common therapeutic method for fibrosis processes, and in order to do this it would be desirable to control all the various types of collagen represented by Types I to IV at the same time.

As effective means for controlling synthesis of various types of collagen molecules simultaneously without losing specificity to collagen, a method for controlling the function of HSP47 can be considered. HSP47 is a collagen-specific molecular chaperone that is essential for intracellular transport and molecular maturation, which are common to synthetic processes for various types of collagen. Therefore, if in stellate cells the function of HSP47 can be controlled specifically, there is a possibility of inhibiting hepatic fibrosis, but there are no reports of such a therapeutic method being attempted.

Certain of the present inventors prepared a ribozyme that specifically controls the function of HSP47 in a cellular system, and showed that the production and secretion of collagens can be controlled by the ribozyme at the same time (Sasaki H, et al. Journal of Immunology, 2002, 168: 5178-83; Hagiwara S, et al. J Gene Med. 2003, 5: 784-94). In order to specifically control the synthesis of HSP47, siRNA, which is easier to optimize than ribozyme, can be employed. The siRNA (small interfering RNAs) used in the present specification is a general term for double-strand RNA used in RNAi (RNA interference). RNAi is a phenomenon in which double-strand RNA (double-strand RNA; dsRNA), which is formed from sense RNA and antisense RNA and is homologous with a given gene, destroys a homologous segment of a transcript (mRNA) of the gene. It was originally exhibited in an experiment using a nematode (Fire A, et al: Nature (1998) 391: 806-811), and it has been shown that a similar induction mechanism is present in mammalian cells (Ui-Tei K, et al: FEBS Lett (2000) 479: 79-82). Furthermore, Elbashir et al. have shown that a short dsRNA having a length of on the order of 21 to 23 bp can induce RNAi in a mammalian cell system without exhibiting cytotoxicity (Elbashir S M, et al: Nature (2001) 411: 494-498). However, in order for the effects of these molecules to be exhibited effectively, it is necessary to employ a method that is specific to a target organ.

### CITATION LIST

Patent Publication 1: Japanese translation 5-503076 of a PCT application
Nonpatent Publication 1: Madro A et al., Med Sci Monit. 2004 July; 10(7): RA166-70
Nonpatent Publication 2: Jaster R, Mol Cancer. 2004 October 6; 3(1): 26
Nonpatent Publication 3: Fallowfield J A, Iredale J P, Expert Opin Ther Targets. 2004 October; 8(5): 423-35
Nonpatent Publication 4: Pinzani M, Rombouts K. Dig Liver Dis. 2004 April; 36(4): 231-42
Nonpatent Publication 5: Qi Z et al., Proc Natl Acad Sci USA. 1999 March 2; 96(5): 2345-9
Nonpatent Publication 6: George J et al., Proc Natl Acad Sci USA. 1999 October 26; 96(22): 12719-24
Nonpatent Publication 7: Ueki K et al., Nat Med. 1999 February; 5(2): 226-30
Nonpatent Publication 8: Iimuro Y et al., Gastroenterology 2003; 124: 445-458
Nonpatent Publication 9: Liu W B et al., World J Gastroenterol. 2003 February; 9(2): 316-9
Nonpatent Publication 10: Marra F et al., Gastroenterology. 2000 August; 119(2): 466-78
Nonpatent Publication 11: Yoshiji H et al., Hepatology. 2001 October; 34(4 Pt 1): 745-50
Nonpatent Publication 12: Liu X J et al., World J Gastroenterol. 2002 August; 8(4): 739-45
Nonpatent Publication 13: Benedetti A et al., Gastroenterology. 2001 February; 120(2): 545-56
Nonpatent Publication 14: Wang L et al., World J Gastroenterol 2004 October 1; 10(19): 2831-2835
Nonpatent Publication 15: Orr J G et al., Hepatology. 2004 July; 40(1): 232-42
Nonpatent Publication 16: Sasaki H et al., Journal of Immunology, 2002, 168: 5178-83
Nonpatent Publication 17: Hagiwara S et al., J Gene Med. 2003, 5: 784-94
Nonpatent Publication 18: Fire A et al.: Nature (1998) 391: 806-811
Nonpatent Publication 19: Ui-Tei K et al.: FEBS Lett (2000) 479: 79-82
Nonpatent Publication 20: Elbashir S M et al.: Nature (2001) 411: 494-498
Nonpatent Publication 21: Yasuhiko Tabata, New Developments in Drug Delivery System DDS Technology and their Application—Cutting-edge technology for biomedical research and advanced medical treatment, Medical Do, ISBN: 4944157932, 2003
Nonpatent Publication 22: Mitsuru Hashida, Drug Delivery Systems—New challenges for drug discovery and therapy, New Bioscience Series, Kagaku-dojin, ISBN: 4759803858, 1995

### SUMMARY OF THE INVENTION

### Problems to be Solved by the Invention

In order to target a tissue and/or an organ, the application of a drug delivery system (DDS) is one effective means (Yasuhiko Tabata, New Developments in Drug Delivery System DDS Technology and their Application—Cutting-edge technology for biomedical research and advanced medical treatment, Medical Do, ISBN: 4944157932, 2003: Mitsuru Hashida, Drug Delivery Systems—New challenges for drug discovery and therapy, New Bioscience Series, Kagaku-dojin, ISBN: 4759803858, 1995). As a drug carrier used in the drug delivery system (DDS), there are those in which a polymer micelle, a liposome, a microemulsion, etc. is applied. As a technique for enhancing the specificity of these carriers toward a target organ, there are known a technique in which an antibody and/or ligand for an organ- and/or tissue-specific antigen or receptor is mixed with or bonded to the carrier, and a technique in which physicochemical properties of the carrier are utilized, but there is no known technique for the particular case in which stellate cells are targeted.

### Means for Solving the Problems

The present invention relates to a drug carrier and a drug carrier kit that enable a diagnostic and/or therapeutic drug to be specifically transported to stellate cells. The drug carrier in the present invention may be in any of polymer micelle, liposome, emulsion, microsphere, and nanosphere form, and by bonding thereto or including therein vitamin A (VA), a retinoid derivative such as, for example, tretinoin, adapalene, or retinol palmitate, or a vitamin A analogue such as, for example, Fenretinide (4-HPR), a therapeutic drug can be transported specifically to hepatic stellate cells. Furthermore, by preparing one in which the drug carrier includes one molecule or a plurality of molecules selected from TGFβ activity inhibitors such as a truncated TGFβ type II receptor and a soluble TGFβ type II receptor, growth factor preparations such as HGF, MMP production promoters such as an MMP gene-containing adenovirus vector, a cell activation inhibitors and/or growth inhibitors including a PPARγ-ligand, an angiotensin-II type I receptor antagonist, a PDGF tyrosine kinase inhibitor, and a sodium channel inhibitor such as amiloride, and apoptosis inducers such as compound 861 and gliotoxin, and by orally, or parenterally, for example, intravenously or intraperitoneally administering it to a patient having a risk of fibrosis or fibrosis symptoms, or patients having various fibrosis-related disorders such as, for example, hepatic cirrhosis, hepatic failure, liver cancer, or chronic pancreatitis, the activation of stellate cells can be suppressed, and fibrosis and/or fibrosis-related disease conditions can be prevented, inhibited, or improved. Alternatively, or in addition thereto, by using the drug carrier which encloses therein a ribozyme, an antisense RNA, or an siRNA that specifically inhibits HSP47, which is a collagen-specific molecular chaperone, or TIMP, which is an MMP inhibitor, secretion of type I to IV collagens can be inhibited simultaneously, and as a result fibrogenesis can be inhibited effectively.

Therefore, in one aspect, the present invention relates to a stellate cell-specific drug carrier having a retinoid derivative and/or a vitamin A analogue as a component. In one embodiment, the drug carrier may comprise a drug carrier component other than the retinoid derivative and/or a vitamin A analogue. In one embodiment, the retinoid derivative may comprises a compound consisting of the structure (retinoid)m-linker-(retinoid)n, wherein m and n are independently 0, 1, 2, or 3, except that m and n are not both zero; and wherein the linker comprises a polyethylene glycol (PEG) or PEG-like molecule. In one embodiment, the retinoid derivative may comprises a compound consisting of the structure (lipid)ₘ-linker-(retinoid)ₙ, wherein m and n are independently 0, 1, 2, or 3, except that m and n are not both zero; and wherein the linker comprises a polyethylene glycol (PEG) molecule.

Furthermore, the present invention relates to the drug carrier wherein the retinoid derivative includes vitamin A.

Moreover, the present invention relates to the drug carrier wherein the retinoid derivative and/or the vitamin A analogue are contained at 0.2 to 20 wt %.

Furthermore, the present invention relates to the drug carrier wherein it is in any one of polymer micelle, liposome, emulsion, microsphere, and nanosphere form.

Moreover, the present invention relates to a medicine for treating a stellate cell-related disorder, the medicine including the drug carrier and a drug for controlling the activity or growth of stellate cells.

Furthermore, the present invention relates to the medicine wherein the disorder is selected from the group consisting of hepatitis, hepatic fibrosis, hepatic cirrhosis, liver cancer, pancreatitis, pancreatic fibrosis, pancreatic cancer, vocal cord scarring, vocal cord mucosal fibrosis, and laryngeal fibrosis.

Moreover, the present invention relates to the medicine wherein the drug for controlling the activity or growth of stellate cells is selected from the group consisting of a TGFβ activity inhibitor, a preparation having HGF activity, an MMP production promoter, a TIMP production inhibitor, a PPARγ ligand, an angiotensin activity inhibitor, a PDGF activity inhibitor, a sodium channel inhibitor, an apoptosis inducer, and an siRNA, ribozyme, antisense nucleic acid, or DNA/RNA chimera polynucleotide, or a vector expressing same, that targets an extracellular matrix constituent molecule produced by stellate cells or one or more molecules having the function of producing or secreting the extracellular matrix constituent molecule.

Furthermore, the present invention relates to the medicine wherein the molecule having the function of producing or secreting the extracellular matrix constituent molecule is HSP47.

Moreover, the present invention relates to the medicine wherein the drug and the drug carrier are mixed at a place of medical treatment or in the vicinity thereof.

Furthermore, the present invention relates to a preparation kit for the medicine, the kit including one or more containers containing one or more of the drug for controlling the activity or growth of stellate cells, a drug carrier constituent, and a retinoid derivative and/or a vitamin A analogue.

Moreover, the present invention relates to a method for treating a stellate cell-related disorder, the method including administering an effective amount of the medicine to a subject in need thereof.

Furthermore, the present invention relates to the method wherein the disorder is selected from the group consisting of hepatitis, hepatic fibrosis, hepatic cirrhosis, liver cancer, pancreatitis, pancreatic fibrosis, pancreatic cancer, vocal cord scarring, vocal cord mucosal fibrosis, and laryngeal fibrosis.

Moreover, the present invention relates to the method wherein the medicine is parenterally administered.

Furthermore, the present invention relates to use of the drug carrier in the production of a medicine for treating a stellate cell-related disorder.

Moreover, the present invention relates to a drug delivery method for stellate cells utilizing the drug carrier.

Furthermore, the present invention also relates to a drug carrier for inhibiting fibrosis that includes a retinoid derivative and/or a vitamin A analogue as a component and transports a drug for controlling the activity or growth of stellate cells specifically to stellate cells, the drug carrier for inhibiting fibrosis wherein the retinoid derivative includes vitamin A, the drug carrier for inhibiting fibrosis wherein the retinoid derivative and/or the vitamin A analogue are contained at 0.2% to 20%, the drug carrier for inhibiting fibrosis wherein it is in any one of polymer micelle, liposome, emulsion, microsphere, and nanosphere form, the drug carrier for inhibiting fibrosis wherein the drug for controlling the activity or growth of stellate cells includes one or more drugs selected from a TGFβ activity inhibitor, a preparation having HGF activity, an MMP production promoter, a TIMP production inhibitor, a PPARγ ligand, an angiotensin activity inhibitor, a PDGF activity inhibitor, a sodium channel inhibitor, and an apoptosis inducer, the drug carrier for inhibiting fibrosis wherein the drug for controlling the activity or growth of stellate cells includes an siRNA, a ribozyme, or an antisense RNA, or a vector expressing same, that targets an extracellular matrix constituent molecule produced by stellate cells, or that targets one or more molecules having the function of producing or secreting the extracellular matrix constituent molecule, and the drug carrier for inhibiting fibrosis wherein the molecule having the function of producing or secreting the extracellular matrix constituent molecule is HSP47.

Moreover, the present invention relates to a drug carrier kit for inhibiting fibrosis that includes one or more containers containing one or more of a drug for controlling the activity or growth of stellate cells, a drug carrier constituent, and a retinoid derivative and/or a vitamin A analogue, the drug carrier kit for inhibiting fibrosis wherein the retinoid derivative includes vitamin A, the drug carrier kit for inhibiting fibrosis wherein the retinoid derivative and/or the vitamin A analogue are contained at 0.2% to 20%, the drug carrier kit for inhibiting fibrosis wherein it is in any one of polymer micelle, liposome, emulsion, microsphere, and nanosphere form, the drug carrier kit for inhibiting fibrosis wherein the drug for controlling the activity or growth of stellate cells includes one or more drugs selected from a TGFβ activity inhibitor, a preparation having HGF activity, an MMP production promoter, a TIMP production inhibitor, a PPARγ ligand, an angiotensin activity inhibitor, a PDGF activity inhibitor, a sodium channel inhibitor, and an apoptosis inducer, the drug carrier kit for inhibiting fibrosis wherein the drug for controlling the activity or growth of stellate cells includes an siRNA, a ribozyme, or an antisense RNA, or a vector expressing same, that targets an extracellular matrix constituent molecule secreted by stellate cells, or that targets one or more molecules having the function of producing or secreting the extracellular matrix constituent molecule, and the drug carrier kit for inhibiting fibrosis wherein the molecule having the function of producing or secreting the extracellular matrix constituent molecule is HSP47.

In another aspect, the present invention provides a compound for facilitating drug delivery to a target cell, consisting of the structure (targeting molecule)ₘ-linker-(targeting molecule)ₙ, wherein the targeting molecule is a retinoid having a specific receptor or activation/binding site on the target cell; wherein m and n are independently 0, 1, 2 or 3; and wherein the linker comprises a polyethylene glycol (PEG) or PEG-like molecule. In an embodiment, m and n are not both zero.

In one embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

In another embodiment, the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the compound is a composition of the formula

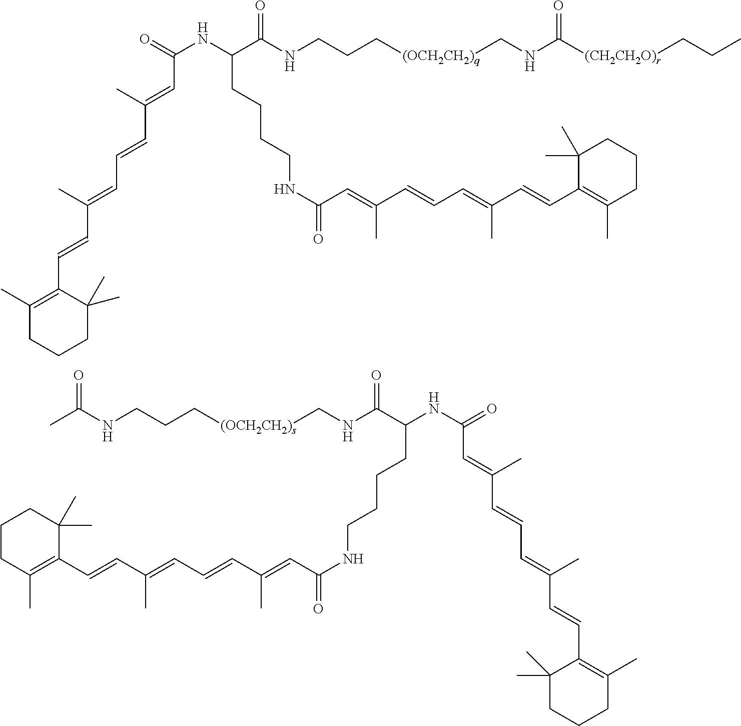

wherein q, r, and s are each independently 1, 2, 3, 4, 5, 6, 7, 8, 9, or 10.

In another embodiment, the formula of the compound comprises

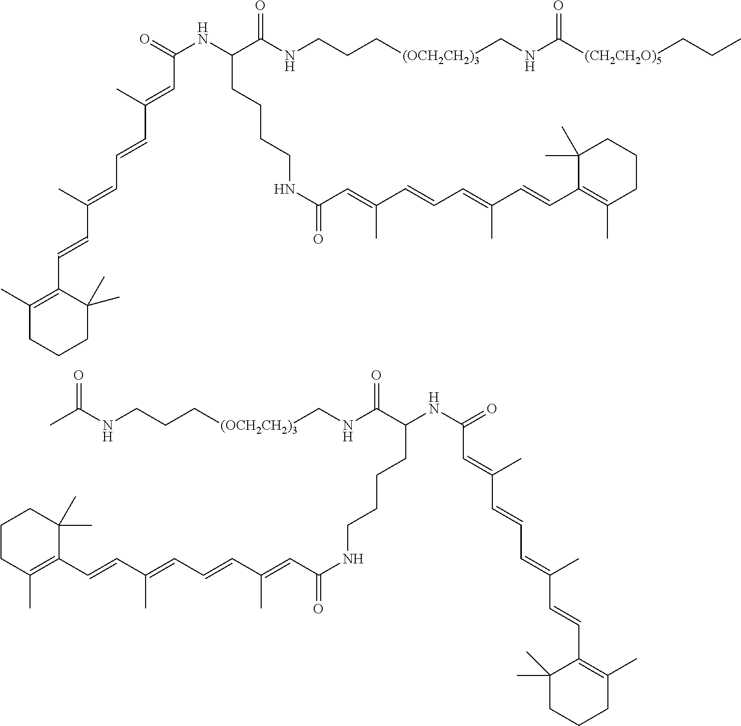

In another aspect, the present invention provides a stellate-cell-specific drug carrier comprising a stellate cell specific amount of a retinoid molecule consisting of the structure (retinoid)ₘ-linker-(retinoid)ₙ; wherein m and n are independently 0, 1, 2 or 3; and wherein the linker comprises a polyethylene glycol (PEG) or PEG-like molecule. In an embodiment, m and n are not both zero.

In another embodiment, the present invention provides a composition comprising a liposomal composition. In other embodiments, the liposomal composition comprises a lipid vesicle comprising a bilayer of lipid molecules.

In certain embodiments, the retinoid molecule is at least partially exposed on the exterior of the drug carrier before the drug carrier reaches the stellate cell.

In another embodiment, the retinoid is 0.1 mol % to 20 mol % of the lipid molecules. The retinoid will be present in a concentration of about 0.3 to 30 weight percent, based on the total weight of the composition or formulation, which is equivalent to about 0.1 to about 10 mol %.

The present invention also provides embodiments where the lipid molecules comprise one or more lipids selected from the group consisting of HEDC, DODC, HEDODC, DSPE, DOPE, and DC-6-14. In another embodiment, the lipid molecules further comprise S104.

In certain embodiments, the drug carrier comprises a nucleic acid.

In other embodiments, the nucleic acid is an siRNA that is capable of knocking down expression of hsp47 mRNA in the stellate cell.

In another aspect, the present invention provides a compound for facilitating drug delivery to a target cell, consisting of the structure (lipid)ₘ-linker-(targeting molecule)ₙ, wherein the targeting molecule is a retinoid or a fat soluble vitamin having a specific receptor or activation/binding site on the target cell; wherein m and n are independently 0, 1, 2 or 3; and wherein the linker comprises a polyethylene glycol (PEG) molecule. In an embodiment, m and n are not both zero.

In one embodiment, the lipid is selected from one or more of the group consisting of DODC, HEDODC, DSPE, DOPE, and DC-6-14.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment of the present invention, the fat-soluble vitamin is vitamin D, vitamin E, or vitamin K.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

In another embodiment the present invention is selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

Accordingly, the present invention provides the following:

(1) A stellate cell-specific drug carrier comprising a retinoid derivative and/or a vitamin A analogue as a component.

(2) The drug carrier according to (1), wherein the retinoid derivative comprises vitamin A.

(3) The drug carrier according to (1), wherein the retinoid derivative and/or the vitamin A analogue are contained at 0.2 to 20 wt %.

(4) The drug carrier according to any one of (1) to (3), wherein it is in any one of polymer micelle, liposome, emulsion, microsphere, and nanosphere form.

(5) A medicine for treating a stellate cell-related disorder, comprising the drug carrier according to any one of (1) to (4), and a drug for controlling the activity or growth of stellate cells.

(6) The medicine according to (5), wherein the disorder is selected from the group consisting of hepatitis, hepatic fibrosis, hepatic cirrhosis, liver cancer, pancreatitis, pancreatic fibrosis, pancreatic cancer, vocal cord scarring, vocal cord mucosal fibrosis, and laryngeal fibrosis.

(7) The medicine according to either (5) or (6), wherein the drug for controlling the activity or growth of stellate cells is selected from the group consisting of a TGFβ activity inhibitor, a preparation having HGF activity, an MMP production promoter, a TIMP production inhibitor, a PPARγ ligand, an angiotensin activity inhibitor, a PDGF activity inhibitor, a sodium channel inhibitor, an apoptosis inducer, and an siRNA, ribozyme, antisense nucleic acid, or DNA/RNA chimera polynucleotide, or a vector expressing same, that targets an extracellular matrix constituent molecule produced by stellate cells or one or more molecules having the function of producing or secreting the extracellular matrix constituent molecule.

(8) The medicine according to (7), wherein the molecule having the function of producing or secreting the extracellular matrix constituent molecule is HSP47.

(9) The medicine according to any one of (5) to (8), wherein the drug and the drug carrier are mixed at a place of medical treatment or in the vicinity thereof.

(10) A preparation kit for the medicine according to any one of (5) to (9), the kit comprising one or more containers containing one or more of the drug for controlling the activity or growth of stellate cells, a drug carrier constituent, and a retinoid derivative and/or a vitamin A analogue.

(11) A method for treating a stellate cell-related disorder, the method comprising administering an effective amount of the medicine according to any one of (5) to (9) to a subject in need thereof.

(12) The method according to (11), wherein the disorder is selected from the group consisting of hepatitis, hepatic fibrosis, hepatic cirrhosis, liver cancer, pancreatitis, pancreatic fibrosis, pancreatic cancer, vocal cord scarring, vocal cord mucosal fibrosis, and laryngeal fibrosis.

(13) The method according to either (11) or (12), wherein the medicine is parenterally administered.

(14) Use of the drug carrier according to any one of (1) to (4) in the production of a medicine for treating a stellate cell-related disorder.

(15) A drug delivery method for stellate cells utilizing the drug carrier according to any one of (1) to (4).

(16) A compound for facilitating drug delivery to a target cell, consisting of the structure (targeting molecule)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) or PEG-like molecule.

(17) The compound of (16), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(18) The compound of (16), wherein the fat-soluble vitamin is vitamin D, vitamin E, or vitamin K.

(19) The compound of (16), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

(20) The compound of (16), wherein the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

(21) The compound of (20), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(22) The compound of (21), wherein the compound is a composition of formula

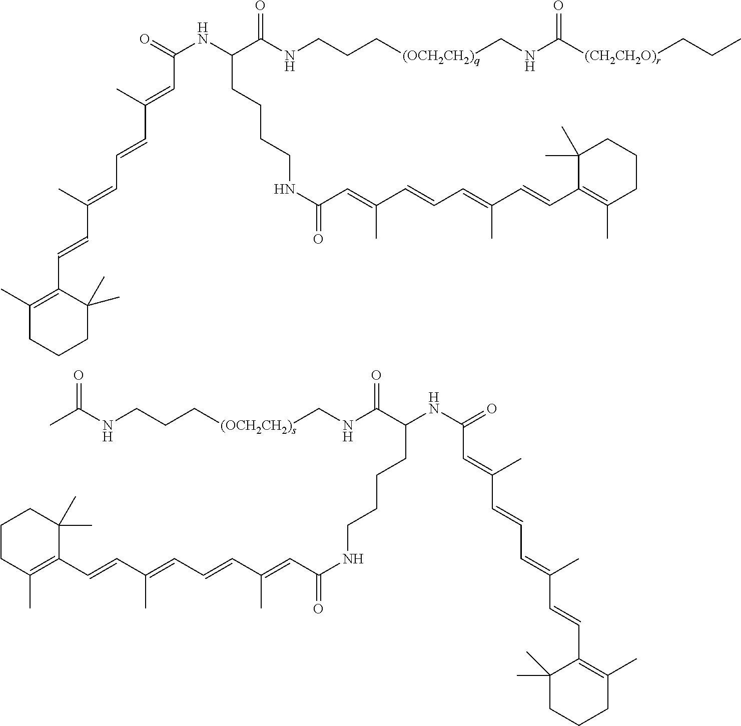

wherein q, r, and s are each independently 1, 2, 3, 4, 5, 6, 7, 8, 9, or 10.

(23) The compound of (21), of the formula

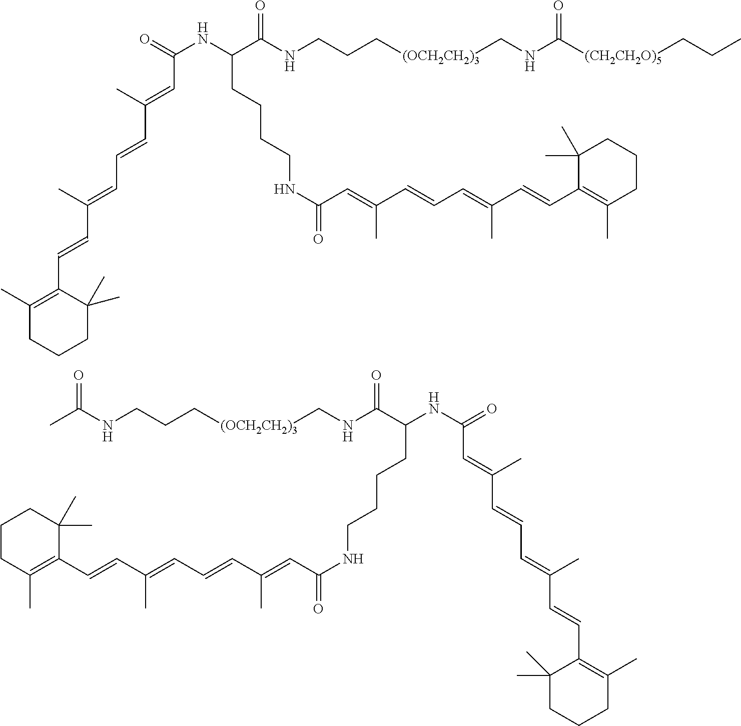

(24) The compound of (16), wherein the PEG is monodisperse.

(25) A stellate-cell-specific drug carrier comprising a stellate cell specific amount of a retinoid molecule consisting of the structure (retinoid)ₘ-linker-(retinoid)ₙ; wherein m and n are independently 0, 1, 2, or 3 (except that m and n are not both zero); and wherein the linker comprises a polyethylene glycol (PEG) or PEG-like molecule.

(26) The drug carrier of (25), further comprising a liposomal composition.

(27) The drug carrier of (26), wherein the liposomal composition comprises a lipid vesicle comprising a bilayer of lipid molecules.

(28) The drug carrier of (26), wherein the retinoid molecule is at least partially exposed on the exterior of the drug carrier before the drug carrier reaches the stellate cell.

(29) The drug carrier of (27), wherein the retinoid is 0.1 mol % to 20 mol % of the lipid molecules.

(30) The drug carrier of (27), wherein the lipid molecules comprise one or more lipids selected from the group consisting of HEDC, DODC, HEDODC, DSPE, DOPE, and DC-6-14.

(31) The drug carrier of (30), wherein the lipid molecules further comprise S104.

(32) The drug carrier of (27), further comprising a nucleic acid.

(33) The drug carrier of (32), wherein the nucleic acid is an siRNA that is capable of knocking down expression of HSP47 mRNA in the stellate cell.

(34) A compound for facilitating drug delivery to a target cell, consisting of the structure (lipid)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) molecule.

(35) The compound of (34), wherein the lipid is selected from one or more of the group consisting of DODC, HEDODC, DSPE, DOPE, and DC-6-14.

(36) The compound of (35), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(37) The compound of (35), wherein the fat-soluble vitamin is vitamin D, vitamin E, or vitamin K.

(38) The compound of (35), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

(39) The compound of (38), selected from the group consisting of D SPE-PEG-VA, D SPE-PEG2000-Glu-VA, D SPE-PEG550-VA, DOPE-VA, DOPE-Glu-VA, DOPE-Glu-NH-VA, DOPE-Gly3-VA, DC-VA, DC-6-VA, and AR-6-VA.

(40) A stellate-cell-specific drug carrier comprising a stellate cell specific amount of a targeting molecule consisting of the molecule (lipid)ₙ-linker-(retinoid)ₙ, wherein n=0, 1, 2 or 3 (except that m and n are not both zero); and wherein the linker comprises a polyethylene glycol (PEG) or PEG-like molecule.

(41) The drug carrier of (40), further comprising a liposomal composition.

(42) The drug carrier of (40), wherein the liposomal composition comprises a lipid vesicle comprising a bilayer of lipid molecules.

(43) The drug carrier of (42), wherein the retinoid molecule is at least partially exposed on the exterior of the drug carrier before the drug carrier reaches the stellate cell.

(44) The drug carrier of (42), wherein the retinoid is 0.2 mol % to 20 mol % of the lipid molecules.

(45) The drug carrier of (44), wherein the lipid molecules comprise one or more lipids selected from the group consisting of HEDC, DODC, HEDC, HEDODC, DSPE, DOPE, and DC.

(46) The drug carrier of (45), wherein the lipid molecules further comprise S104.

(47) The drug carrier of (42), further comprising a nucleic acid.

(48) The drug carrier of (47), wherein the nucleic acid is an siRNA that is capable of knocking down expression of hsp47 mRNA in the stellate cell.

### Effects of the Invention

By the use of the drug carrier and the drug carrier kit of the present invention that enable a diagnostic and/or therapeutic drug to be transported specifically to stellate cells as effective means for preventing, suppressing, or improving fibrosis and/or various types of fibrosis-related disorders, innovative therapeutic effects such as shown by Examples can be provided. That is, since the drug carrier and the drug carrier kit of the present invention specifically target stellate cells, clinical conditions that develop mainly due to stellate cells such as, for example, fibrosis, can be inhibited efficiently and effectively while minimizing side effects.

### BRIEF DESCRIPTION OF THE DRAWINGS

FIG. 1: A diagram showing a protocol with respect to assessment of the effect of gp46-siRNA in vitro using NRK cells, and determination of optimal sequence, timing, and concentration.

FIG. 2: A photographic diagram showing the result of western blotting of gp46 and actin (24 hour culturing, examination of optimal sequence).

FIG. 3: A photographic diagram showing the result of western blotting of gp46 and actin (24 hour culturing, examination of optimal concentration).

FIG. 4: A photographic diagram showing the result of western blotting of gp46 and actin (concentration 50 nM, examination of optimal culturing time).

FIG. 5: A diagram showing a protocol for evaluating inhibition of expression of collagen by gp46-siRNA in NRK cells.

FIG. 6: A graph showing inhibition of collagen synthesis by siRNA.

FIG. 7: A photographic diagram showing HSC-specific siRNA transfection.

FIG. 8: A photographic diagram for evaluating HSC-specific siRNA transfection percentage.

FIG. 9: A photographic diagram for evaluating inhibition of expression of gp46 by siRNA.

FIG. 10: A photographic diagram showing azan staining of rat liver to which DMN had been administered.

FIG. 11: A diagram showing an LC rat treatment protocol.

FIG. 12: A photographic diagram showing azan staining of LC rat liver to which VA-Lip-gp46siRNA had been administered.

FIG. 13: A diagram showing a method for extracting a stained portion by means of NIH Image (6 positions being randomly taken from an azan-stained image).

FIG. 14: A graph showing the ratio by area occupied by fibrotic portions in liver histology (Collagen ratio by area, %).

FIG. 15: A graph showing the amount of hydroxyproline in hepatic tissue.

FIG. 16: A graph showing a survival curve for hepatic cirrhosis rat to which VA-Lip-gp46siRNA had been intraportally administered.

FIG. 17: A photographic diagram showing azan staining of hepatic tissue of hepatic cirrhosis rat to which VA-Lip-gp46siRNA had been intraportally administered.

FIG. 18: A graph showing a survival curve for hepatic cirrhosis rat to which VA-Lip-gp46siRNA had been intraportally administered.

FIG. 19: A photographic diagram showing azan staining of hepatic tissue of hepatic cirrhosis rat to which VA-Lip-gp46siRNA had been intraportally administered.

FIG. 20: A graph showing a survival curve for hepatic cirrhosis rat to which VA-Lip-gp46siRNA had been intravenously administered.

FIG. 21: A graph showing a survival curve for hepatic cirrhosis rat to which VA-Lip-gp46siRNA had been intravenously administered.

FIG. 22: A photographic diagram showing azan staining of hepatic tissue of hepatic cirrhosis rat to which VA-Lip-gp46siRNA had been intravenously administered.

FIG. 23: A diagram showing improvement of VA-Lip-gp46siRNA transfection efficiency by RBP.

FIG. 24: A diagram showing inhibition of VA-Lip-gp46siRNA transfection by anti-RBP antibody.

FIG. 25: VA-conjugate addition to liposomes via decoration enhances siRNA activity

FIG. 26: VA-conjugate addition to liposomes via co-solubilization enhances siRNA activity

FIG. 27: VA-conjugate addition to liposomes via co-solubilization enhances siRNA activity

FIG. 28: VA-conjugate addition to lipoplexes via co-solubilization enhance siRNA activity

FIG. 29: VA-conjugate addition to lipoplexes via co-solubilization vs. decoration.

FIG. 30: in vivo efficacy in mouse, CCl₄ model

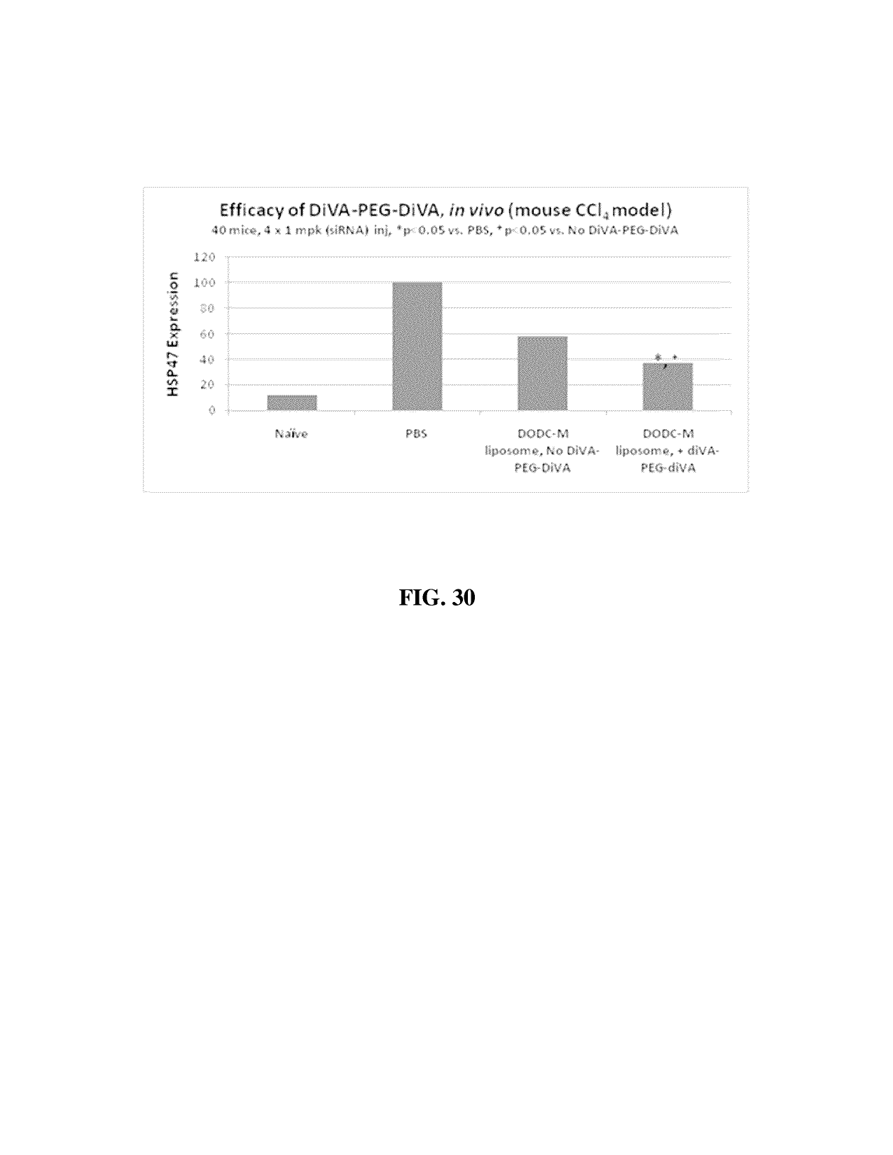

FIG. 31: in vivo efficacy of decorated vs. co-solubilized retinoid conjugates

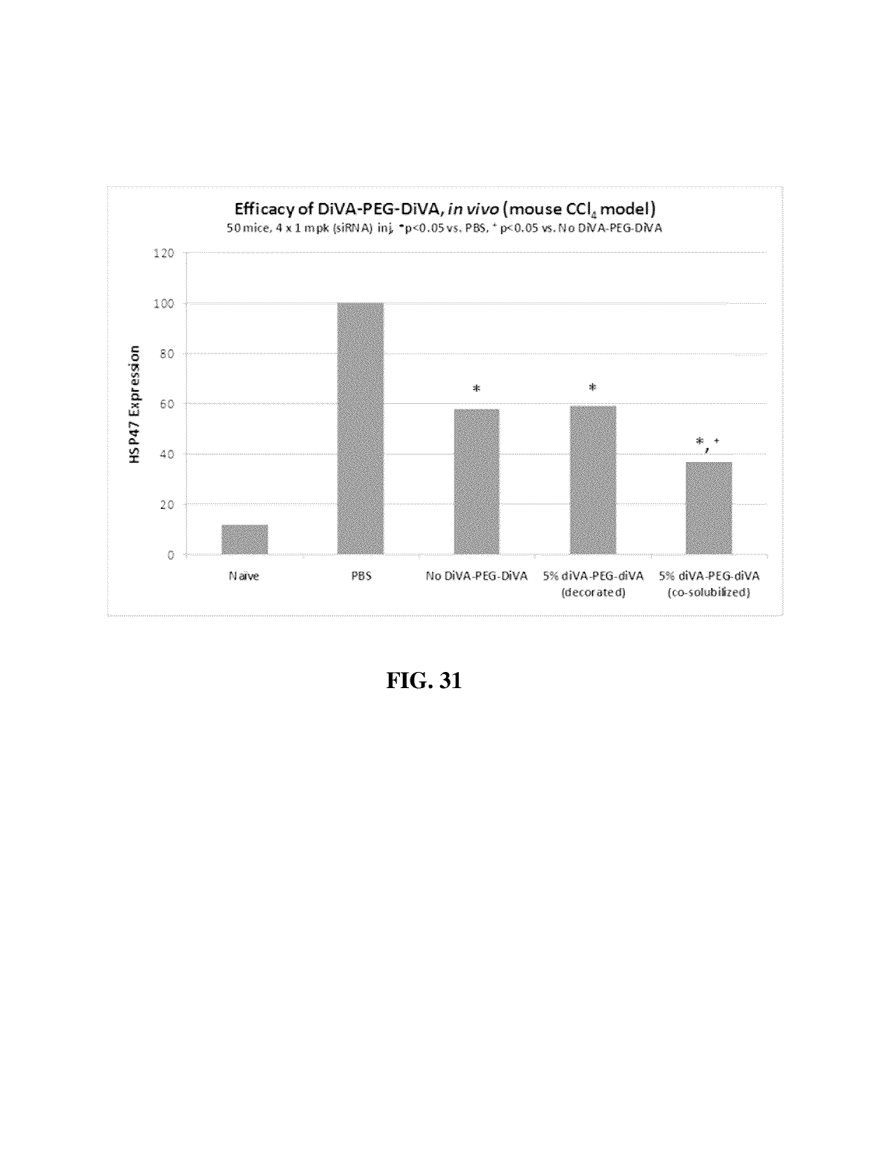

FIG. 32: in vitro efficacy (pHSC), effect of retinoid conjugates in liposome formulations.

FIG. 33: Correlation of retinoid conjugate content (mol %) to in vivo (rat DMNQ) efficacy. Male Sprague-Dawley rats injected intravenously either with formulations containing 0, 0.25, 0.5, 1, and 2% DiVA at a dose of 0.75 mg/kg siRNA, or PBS (vehicle), one hour after the last injection of DMN.

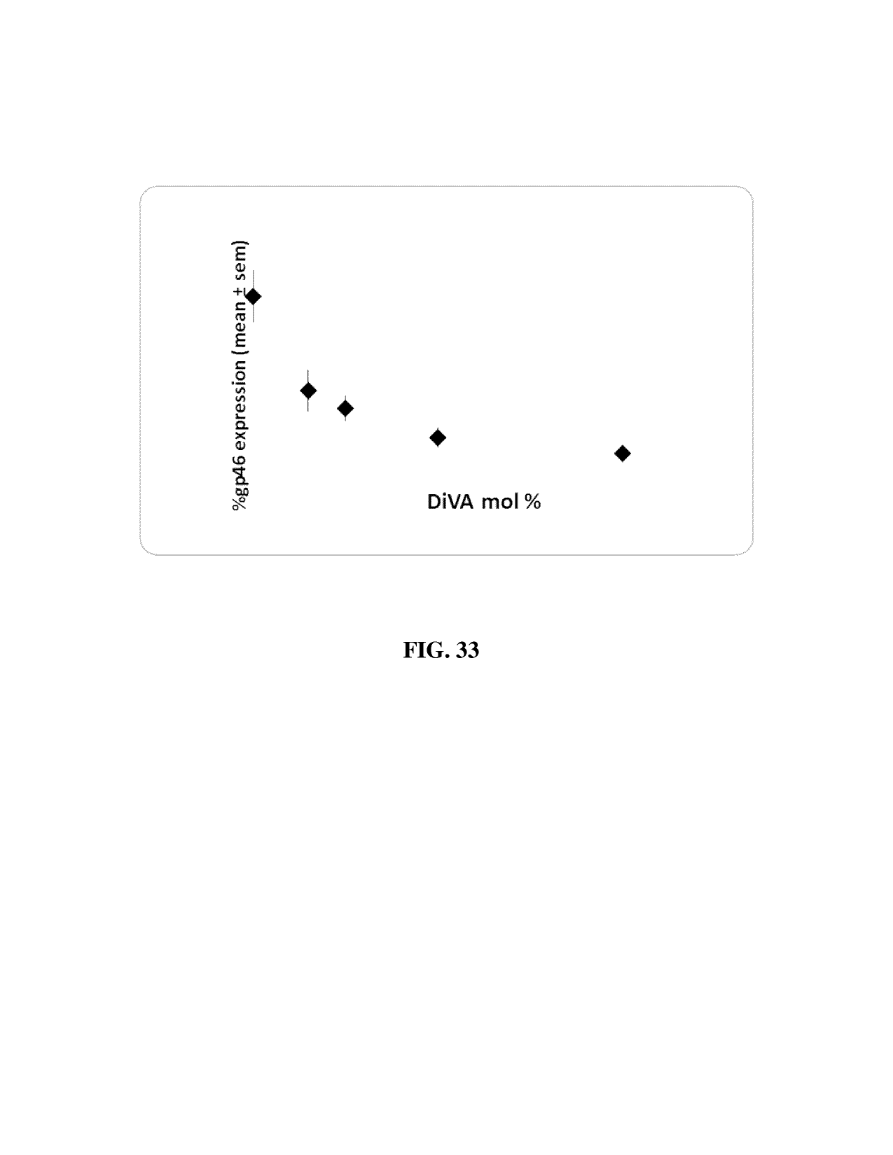

### DETAILED DESCRIPTION OF THE PREFERRED EMBODIMENTS

Within the scope of the invention is a compound for facilitating drug delivery to a target cell, consisting of the structure (targeting molecule)ₘ-linker-(targeting molecule)ₙ, wherein the targeting molecule is a retinoid or a fat soluble vitamin having a specific receptor (or activation/binding site) on the target cell; and wherein m and n are independently 0, 1, 2, or 3 (except that m and n are not both zero); and wherein the linker comprises a polyethylene glycol (PEG) or PEG-like molecule and is designated “Formula A”.

The invention also includes a compound for facilitating drug delivery to a target cell, consisting of the structure (lipid)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) PEG-like molecule and is designated “Formula B”.

It has now been discovered that the compounds of Formula A or Formula B impart properties to the formulations of the invention not previously seen. Formulations of the invention that include compounds of Formula A or Formula B result in superior reduction in gene expression, as compared to formulations that do not include these compounds. Particularly surprising is the ability of formulations of the invention that include compounds of Formula A to reduce the expression of HSP47.

In certain preferred embodiments, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

Preferred embodiments include compounds where the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH. In other embodiments, the PEG is monodisperse.

Another embodiment provides a compound where Formula A is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

In another preferred embodiment, the compound is of the formula

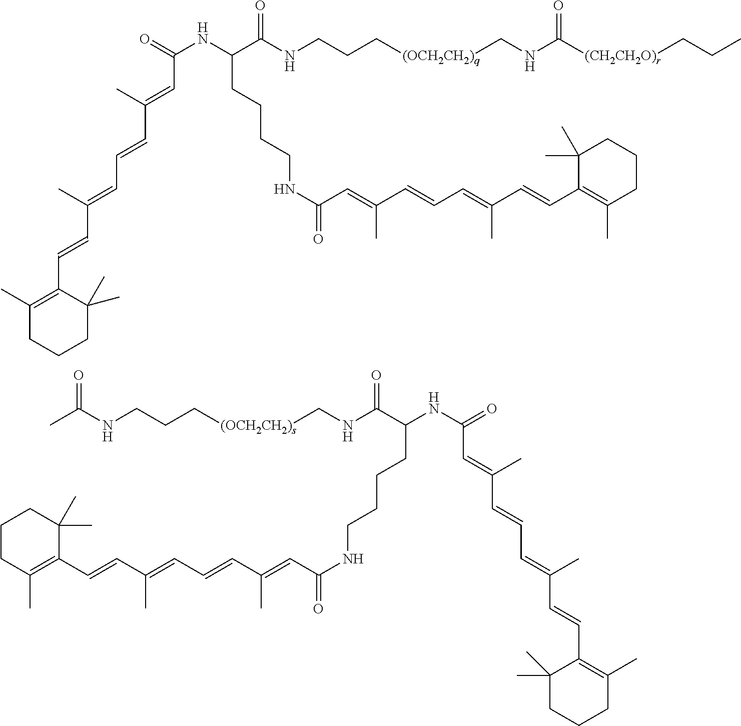

wherein q, r, and s are each independently 1, 2, 3, 4, 5, 6, 7, 8, 9, or 10.

In other preferred embodiments, the formula of the compound comprises

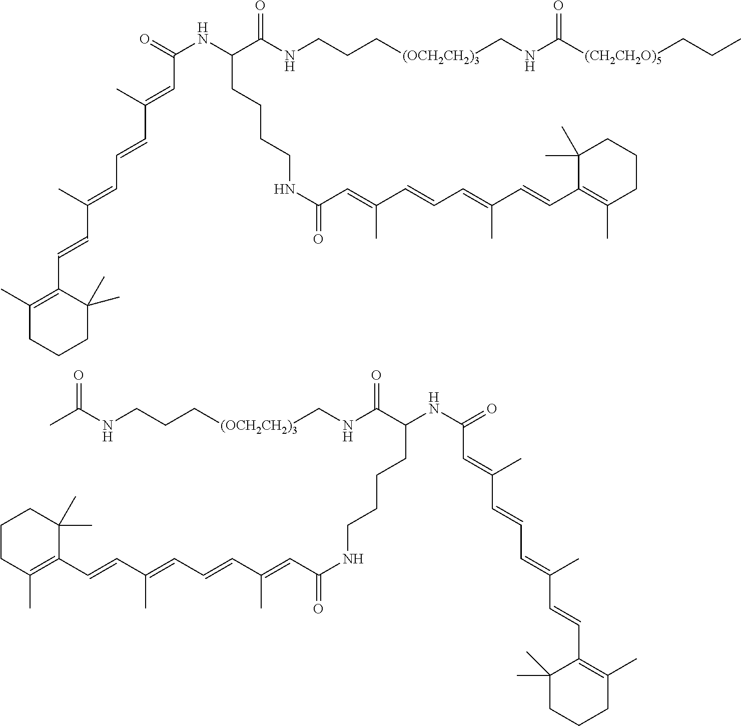

Other embodiments of the invention include the structures shown in Table 1.

TABLE 2

| Lipid Name | Compound Structure |
|---|---|
| SatDiVA | 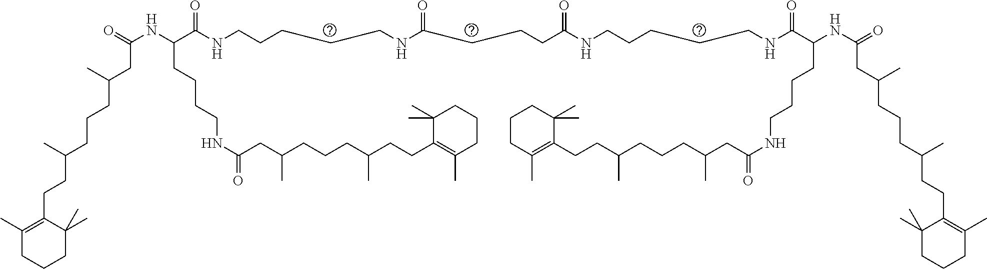 |
| SimDiVA | 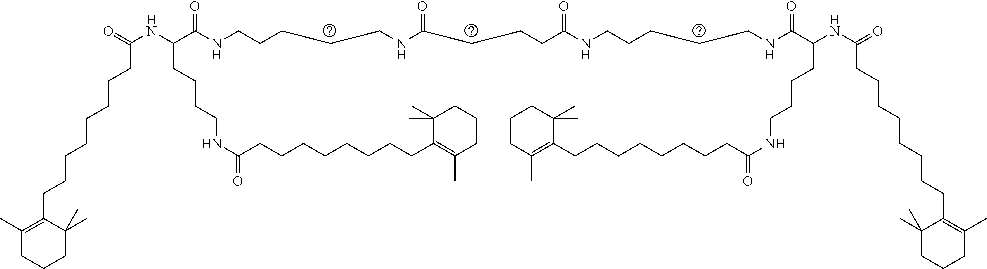 |
| DiVA-PEG18 | 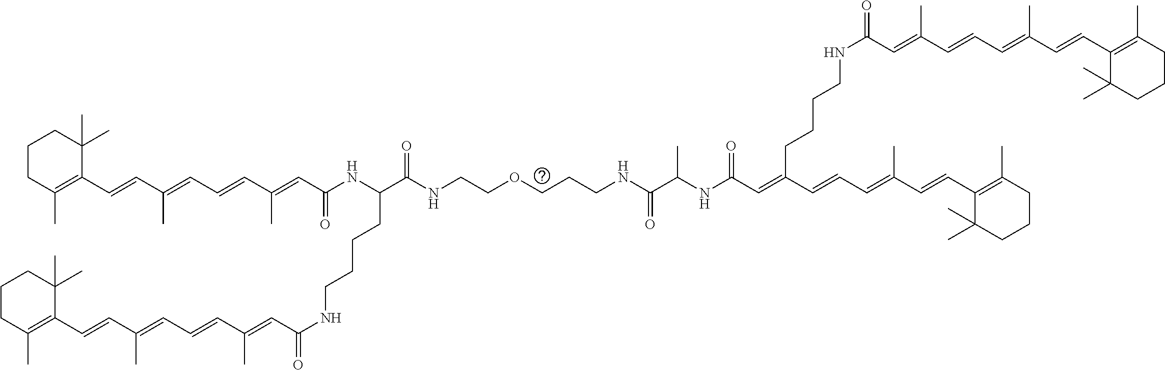 [SMILES: CC1=C(/C=C/C(C)=C/C=C/C(C)=C/C(=O)NCCCCC(/C=C/C=C(C)/C=C/C2=C(C)CCCC2(C)C)=C\C(=O)NC(C)C(=O)NCCCOCCNC(=O)C(CCCCNC(=O)/C=C(C)/C=C/C=C(C)/C=C/C2=C(C)CCCC2(C)C)NC(=O)/C=C(C)/C=C/C=C(C)/C=C/C2=C(C)CCCC2(C)C)C(C)(C)CCC1] |
| TriVA | 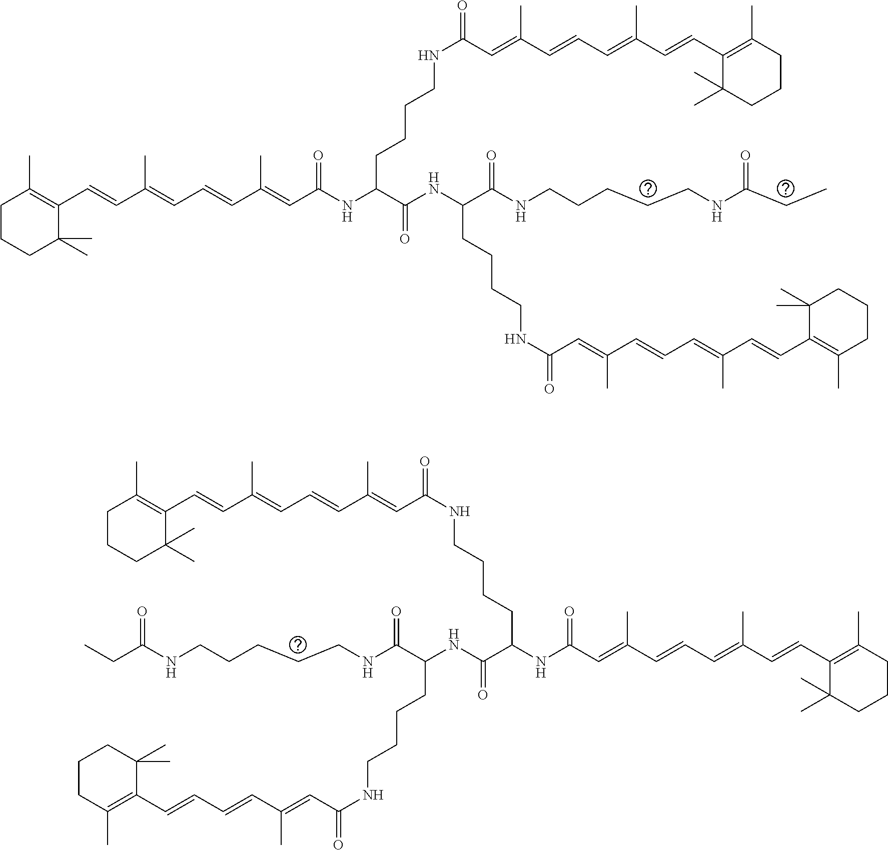 [SMILES: CCC(=O)NCCCCCNC(=O)C(CCCCNC(=O)/C=C(C)/C=C/C=C(C)/C=C/C1=C(C)CCCC1(C)C)NC(=O)C(CCCCNC(=O)/C=C(C)/C=C/C=C(C)/C=C/C1=C(C)CCCC1(C)C)NC(=O)/C=C(C)/C=C/C=C(C)/C=C/C1=C(C)CCCC1(C)C ; CCC(=O)NCCCCCNC(=O)C(CCCCNC(=O)/C=C(C)/C=C/C=C/C1=C(C)CCCC1(C)C)NC(=O)C(CCCCNC(=O)/C=C(C)/C=C/C=C(C)/C=C/C1=C(C)CCCC1(C)C)NC(=O)/C=C(C)/C=C/C=C(C)/C=C/C1=C(C)CCCC1(C)C] |
| 4TTNPB | 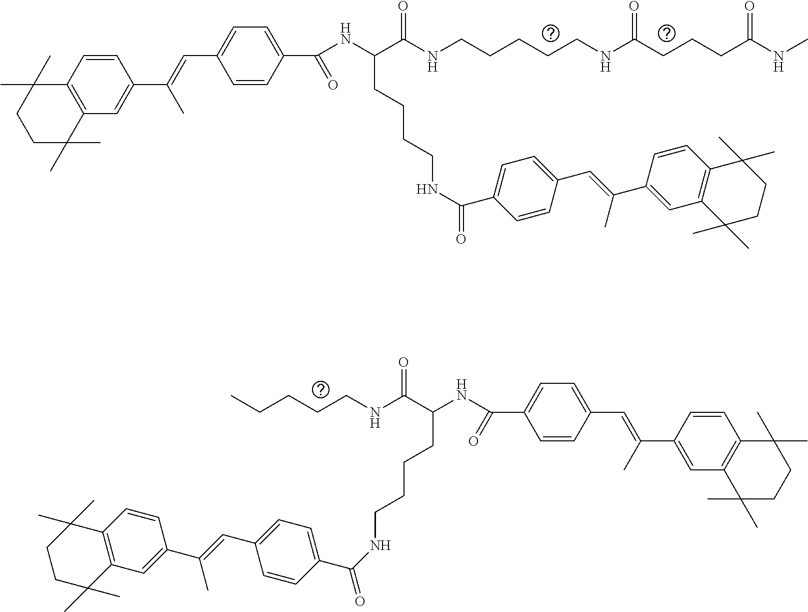 |
| 4Myr | 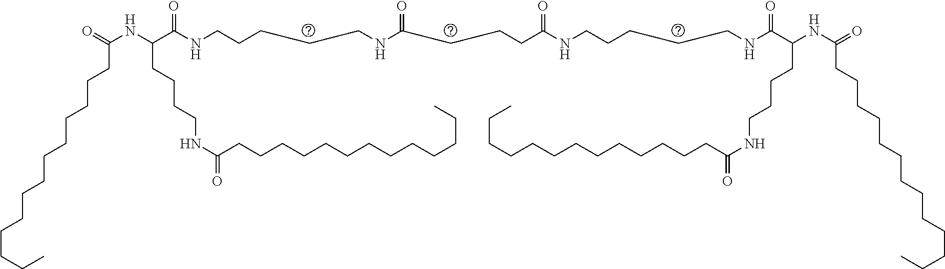 [SMILES: CCCCCCCCCCCCCC(=O)NCCCCC(NC(=O)CCCCCCCCCCCCC)C(=O)NCCCCCNC(=O)CCCC(=O)NCCCCCNC(=O)C(CCCCNC(=O)CCCCCCCCCCCCC)NC(=O)CCCCCCCCCCCCC] |
| DiVA-242 | 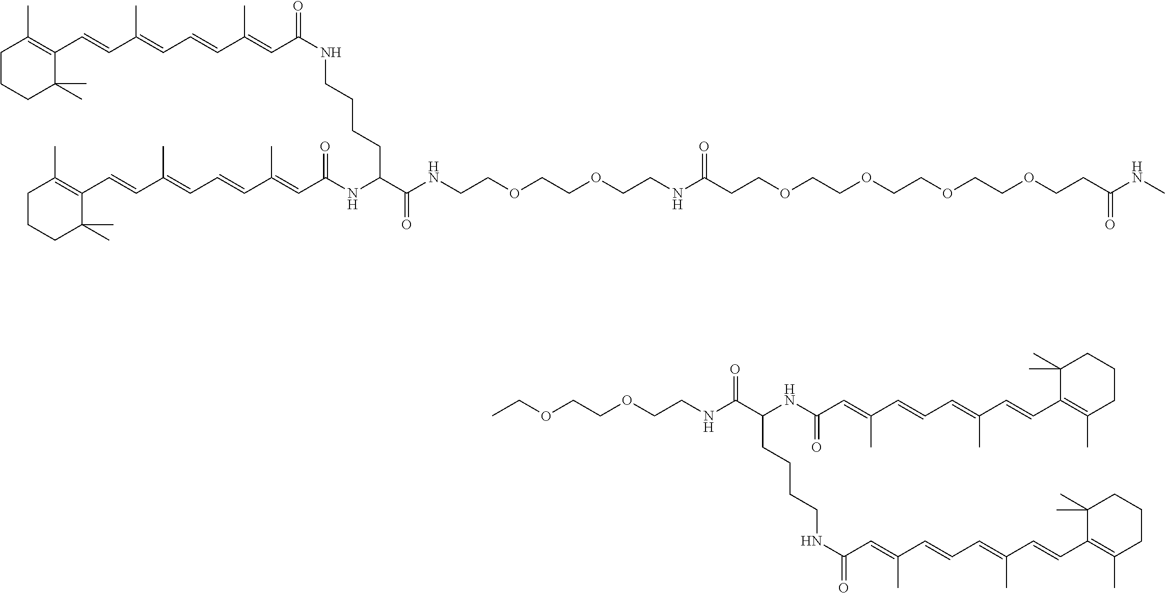 |

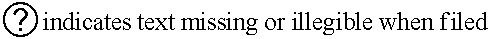

Also within the scope of the invention are formulations comprising at least one compound of Formula A or B and siRNA. It is envisioned that any siRNA molecule can be used within the scope of the invention. Examples of siRNA include:

(SEQ. ID. NO. 1)
Sense (5′->3′) GGACAGGCCUCUACAACUATT
(SEQ. ID. NO. 2)
Antisense (3′->5′) TTCCUGUCCGGAGAUGUUGAU
(SEQ. ID. NO. 3)
Sense (5′->3′) GGACAGGCCUGUACAACUATT
(SEQ. ID. NO. 4)
Antisense (3′->5′) TTCCUGUCCGGACAUGUUGAU

Also within the scope of the invention is a stellate cell-specific drug carrier having a retinoid derivative and/or a vitamin A analogue as a component. The retinoid derivative and/or vitamin A analogue in the present invention includes vitamin A as well as a retinoid derivative or vitamin A analogue in a state in which it is dissolved in or mixed with a medium that can dissolve or retain it.

Any retinoid derivative and/or vitamin A analogue may be used in the present invention as long as it is actively accumulated by stellate cells; examples of the retinoid derivative include, but are not limited to, tretinoin, adapalene, retinol palmitate, the compound of Formula A wherein the targeting molecule is a retinoid, the compound of Formula B wherein the targeting molecule is a retinoid, and in particular vitamin A (retinoic acid), and examples of the vitamin A analogue include, but are not limited to, Fenretinide (4-HPR). The present invention utilizes the property of stellate cells to positively incorporate a retinoid derivative and/or a vitamin A analogue, and by using the retinoid derivative and/or vitamin A analogue as a drug carrier or by bonding to or being included in another drug carrier component, a desired material or body is transported specifically to stellate cells.

The drug carrier of the present invention therefore may contain a drug carrier component other than the retinoid derivative and/or vitamin A analogue. Such a component is not particularly limited, and any component known in the fields of medicine and pharmacy may be used, but it is preferable for it to be capable of including the retinoid derivative and/or vitamin A analogue or bonding thereto. Examples of such a component include a lipid, for example, a phospholipid such as glycerophospholipid, a sphingolipid such as sphingomyelin, a sterol such as cholesterol, a vegetable oil such as soybean oil or poppy seed oil, mineral oil, and a lecithin such as egg-yolk lecithin, but the examples are not limited thereto. Among them, those that can form a liposome are preferable, for example, natural phospholipids such as lecithin, semisynthetic phospholipids such as dimyristoylphosphatidylcholine (DMPC), dipalmitoylphosphatidylcholine (DPPC), and distearoylphosphatidylcholine (DSPC), and cholesterol.

Furthermore, the drug carrier of the present invention may contain a substance that improves incorporation into stellate cells, for example, retinol-binding protein (RBP).

The bonding or inclusion of the retinoid derivative and/or vitamin A analogue with the drug carrier of the present invention may also be carried out by bonding or including the retinoid derivative and/or vitamin A analogue with another component of the drug carrier by chemical and/or physical methods. Alternatively, bonding or inclusion of the retinoid derivative and/or vitamin A analogue with the drug carrier of the present invention may also be carried out by mixing the retinoid derivative and/or vitamin A analogue having formation-affinity and basic components of the drug carrier, into the drug carrier components during preparation of the drug carrier. The amount of retinoid derivative and/or vitamin A analogue bonded to or included in the drug carrier of the present invention may be 0.01% to 100% as a ratio by weight relative to the drug carrier components, preferably 0.2% to 20%, and more preferably 1% to 5%.

The drug carrier of the present invention may be in any form as long as a desired material or body can be transported to target stellate cells, and examples of the form include, but are not limited to, polymer micelle, liposome, emulsion, microsphere, and nanosphere. Furthermore, the drug carrier of the present invention may include in its interior the substance that is to be transported, be attached to the exterior of the substance that is to be transported, or be mixed with the substance that is to be transported as long as the retinoid derivative and/or vitamin A analogue included therein is at least partially exposed on the exterior of the preparation before it reaches the stellate cells at the latest.

One embodiment includes a stellate-cell-specific drug carrier comprising a liposomal composition. The liposomal composition can comprise a lipid vesicle comprising a bilayer of lipid molecules. In certain embodiments it may preferred that the retinoid derivative and/or vitamin A analogue molecule is at least partially exposed on the exterior of the drug carrier before the drug carrier reaches the stellate cell.

Certain embodiments of the present invention provide that the lipid molecules comprise one or more lipids selected from the group consisting of HEDC, DODC, HEDODC, DSPE, DOPE, and DC-6-14. In other embodiments, the lipid molecules can further comprise S104.

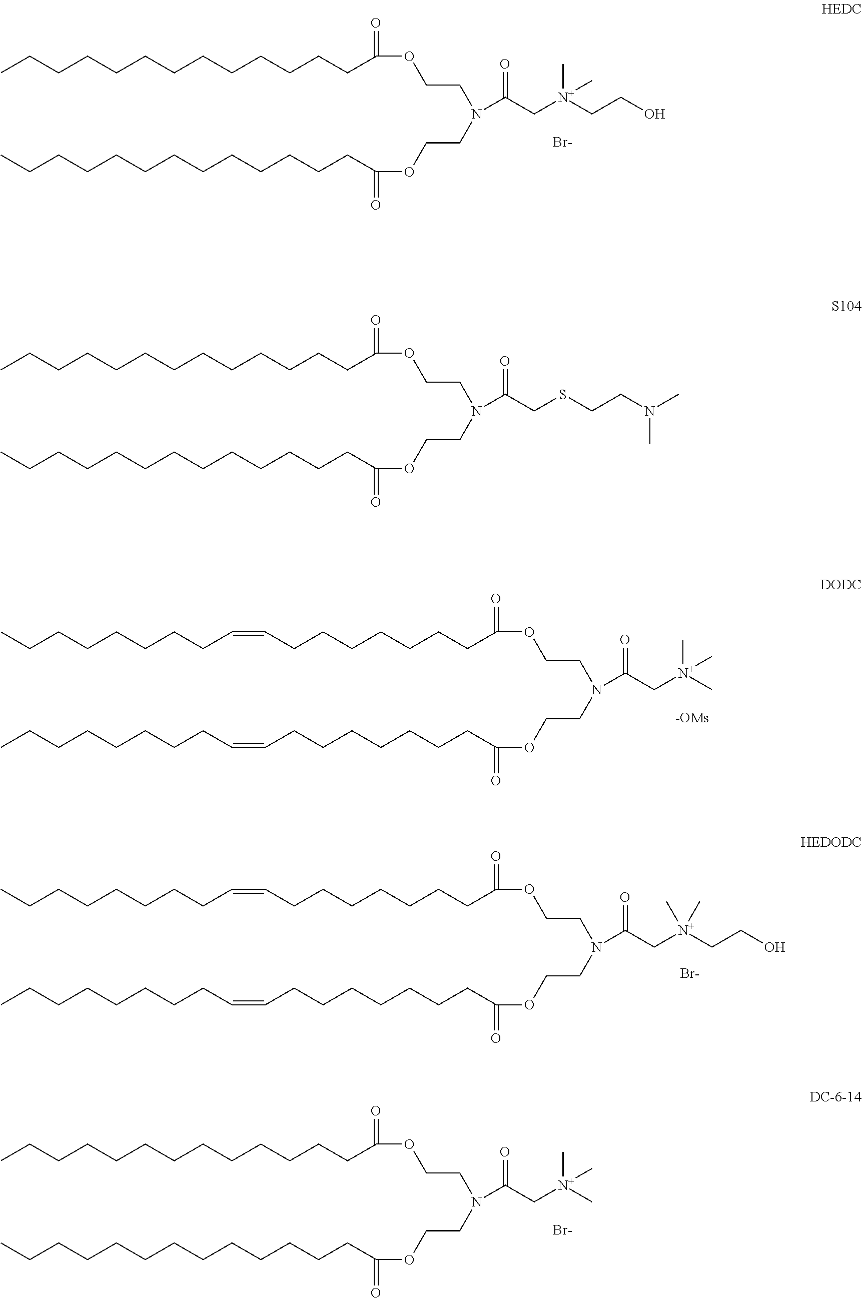

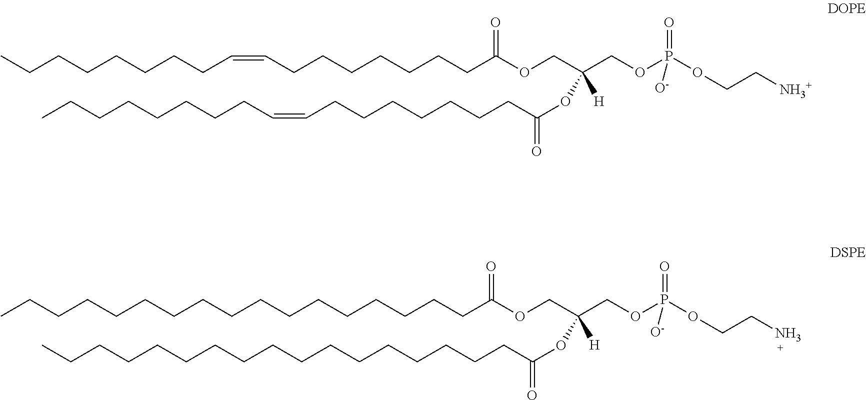 [SMILES: CCCCCCCC/C=C\CCCCCCCC(=O)OC[C@H](COP(=O)([O-])OCC[NH3+])OC(=O)CCCCCCC/C=C\CCCCCCCC ; CCCCCCCCCCCCCCCCCC(=O)OC[C@H](COP(=O)([O-])OCCN)OC(=O)CCCCCCCCCCCCCCCCC]

In some embodiments, the siRNA will be encapsulated by the liposome so that the siRNA is inaccessible to the aqueous medium. In other embodiments, the siRNA will not be encapsulated by the liposome. In such embodiments, the siRNA can be complexed on the outer surface of the liposome. In these embodiments, the siRNA is accessible to the aqueous medium.

Other embodiments include a stellate-cell-specific drug carrier comprising a liposomal composition. The liposomal composition can comprise a lipid vesicle comprising a bilayer of lipid molecules. In other embodiments, the retinoid derivative and/or vitamin A analogue molecule is at least partially exposed on the exterior of the drug carrier before the drug carrier reaches the stellate cell.

In certain preferred embodiments, the retinoid derivative and/or vitamin A analogue is 0.1 mol % to 20 mol % of the lipid molecules.

The forgoing compositions can also include PEG-conjugated lipids, which are known in the art per se, including PEG-phospholipids and PEG-ceramides, including one or more molecules selected from the following: PEG2000-DSPE, PEG2000-DPPE, PEG2000-DMPE, PEG2000-DOPE, PEG1000-DSPE, PEG1000-DPPE, PEG1000-DMPE, PEG1000-DOPE, PEG550-D SPE, PEG550-DPPE, PEG-550DMPE, PEG-1000DOPE, PEG-cholesterol, PEG2000-ceramide, PEG1000-ceramide, PEG750-ceramide, and PEG550-ceramide.

The foregoing compositions of the invention can include one or more phospholipids such as, for example, 1,2-distearoyl-sn-glycero-3-phosphocholine (“DSPC”), dipalmitoylphosphatidylcholine (“DPPC”), 1,2-dipalmitoyl-sn-glycero-3-phosphoethanolamine (“DPPE”), and 1,2-dioleoyl-sn-glycero-3-phosphoethanolamine (“DOPE”). Preferably, the helper phospholipid is DOPE.

The drug carrier of the present invention specifically targets stellate cells and enables a desired effect such as, for example, inhibition or prevention of fibrosis to be exhibited with the maximum effect and minimum side effects by efficiently transporting to stellate cells a desired material or body such as, for example, a drug for controlling the activity or growth of stellate cells. The material or body that the present drug carrier delivers is not particularly limited, but it preferably has a size that enables physical movement in a living body from an administration site to the liver, pancreas, etc., where stellate cells are present. The drug carrier of the present invention therefore can transport not only a material such as an atom, a molecule, a compound, a protein, or a nucleic acid but also a body such as a vector, a virus particle, a cell, a drug release system constituted from one or more elements, or a micromachine. The material or body preferably has the property of exerting some effect on stellate cells, and examples thereof include one that labels stellate cells and one that controls the activity or growth of stellate cells.

Therefore, in one embodiment of the present invention, it is a ‘drug for controlling the activity or growth of stellate cells’ that the drug carrier delivers. This may be any drug that directly or indirectly inhibits the physicochemical actions of stellate cells involved in the promotion of fibrosis, and examples thereof include, but are not limited to, TGFβ activity inhibitors such as a truncated TGFβ type II receptor and a soluble TGFβ type II receptor, growth factor preparations such as HGF and expression vectors therefor, MMP production promoters such as an MMP gene-containing adenovirus vector, TIMP production inhibitors such as an antisense TIMP nucleic acid, a PPARγ ligand, cell activation inhibitors and/or cell growth inhibitors such as an angiotensin activity inhibitor, a PDGF activity inhibitor, and a sodium channel inhibitor, and also apoptosis inducers such as compound 861 and gliotoxin, adiponectin (JP, A, 2002-363094), and a compound having Rho kinase inhibitory activity such as (+)-trans-4-(1-aminoethyl)-1-(4-pyridylcarbamoyl)cyclohexane (WO 00/64478). Furthermore, the ‘drug for controlling the activity or growth of stellate cells’ in the present invention may be any drug that directly or indirectly promotes the physicochemical actions of stellate cells directly or indirectly involved in the inhibition of fibrosis, and examples thereof include, but are not limited to, a drug for promoting a collagen degradation system, e.g., MMP production promoters such as an MMP expression vector, HGF, and drugs having HGF-like activity such as HGF analogues and expression vectors therefor.

Other examples of the ‘drug for controlling the activity or growth of stellate cells’ in the present invention include a drug for controlling the metabolism of an extracellular matrix such as collagen, for example, a substance having an effect in inhibiting the expression of a target molecule, such as siRNA, ribozyme, and antisense nucleic acid (including RNA, DNA, PNA, and a composite thereof), a substance having a dominant negative effect, and vectors expressing same, that target, for example, an extracellular matrix constituent molecule produced by stellate cells or target one or more molecules that have the function of producing or secreting the extracellular matrix constituent molecule.

The siRNA is a double-strand RNA having a sequence specific to a target molecule such as an mRNA, and promotes degradation of the target molecule, thus inhibiting expression of a material formed thereby such as, for example, a protein (RNA interference). Since the principle was published by Fire et al. (Nature, 391: 806-811, 1998), a wide range of research has been carried out into the optimization of siRNA, and a person skilled in the art is familiar with such techniques. Furthermore, materials other than siRNA that cause RNA interference or another gene expression inhibition reaction have been intensively investigated, and there are currently a large number of such materials.

For example, JP, A, 2003-219893 describes a double-strand polynucleotide formed from RNA and DNA that inhibits the expression of a target gene. This polynucleotide may be a DNA/RNA hybrid in which one of two strands is DNA and the other is RNA, or a DNA/RNA chimera in which one portion of the same strand is DNA and the other portion is RNA. Such a polynucleotide is preferably formed from 19 to 25 nucleotides, more preferably 19 to 23 nucleotides, and yet more preferably 19 to 21 nucleotides; in the case of the DNA/RNA hybrid, it is preferable that the sense strand is DNA and the antisense strand is RNA, and in the case of the DNA/RNA chimera, it is preferable that one portion on the upstream side of the double-strand polynucleotide is RNA. Such a polynucleotide may be prepared so as to have any sequence in accordance with a chemical synthetic method known per se.

With regard to the target molecule, for example, a molecule that can inhibit the secretion of all extracellular matrix constituent molecules together is preferable, and examples of such a molecule include, but are not limited to, HSP47. HSP47 or a homologous gene sequence thereof is disclosed as, for example, GenBank accession No. AB010273 (human), X60676 (mouse), or M69246 (rat, gp46).

Preferred examples of the material that is transported by the drug carrier of the present invention include an siRNA, a DNA/RNA hybrid or chimera polynucleotide, and an antisense nucleic acid, that targets HSP47.

Examples of a material that is delivered by the drug carrier of the present invention include a drug for inhibiting fibrosis such as, for example, G-CSF (WO 2005/082402), a thrombomodulin-like protein (JP, A, 2002-371006), and keratan sulfate oligosaccharide (JP, A, 11-269076).

The material or body that is delivered by the drug carrier of the present invention may or may not be labeled. Labeling is useful at the testing and research level in particular since the feasibility of transport or an increase or decrease in stellate cells can be monitored. A label may be selected from those known to a person skilled in the art; for example, any radioactive isotope, a material that can bond to a material to be labeled (e.g. an antibody), a fluorescent material, a fluorophore, a chemiluminescent material, and an enzyme.

Also within the scope of the invention are pharmaceutical formulations that include any of the aforementioned compounds in addition to a pharmaceutically acceptable carrier or diluent. Pharmaceutical formulations of the invention will include at least one therapeutic agent. Preferably, the therapeutic agent is an siRNA. It is envisioned that any siRNA molecule can be used within the scope of the invention. As previously described, siRNA include the sequences shown as SEQ ID NO: 1, SEQ ID NO: 2, SEQ ID NO: 3, and SEQ ID NO: 4.

In preferred formulations of the invention including siRNA, the siRNA is encapsulated by the liposome. In other embodiments, the siRNA can be outside of the liposome. In those embodiments, the siRNA can be complexed to the outside of the liposome.

A useful range of cationic lipid: siRNA (lipid nitrogen to siRNA phosphate ratio, “N:P”) is 0.2 to 5.0. A particularly preferred range of N:P is 1.5 to 2.5 for compositions and formulations of the description.

Preferred formulations of the invention include those comprising HEDC: S104:DOPE: Cholesterol:PEG-DMPE:DiVA-PEG-DiVA (20:20:30:25:5:2 molar ratio) and HEDC: S104:DOPE: Cholesterol:PEG-DMPE:DiVA-PEG-DiVA (20:20:30:25:5:2 molar ratio) wherein DiVA-PEG-DiVA is co-solubilized. DODC:DOPE:cholesterol:PEG-lipid:DiVA-PEG-DiVA (50:10:38:2:5 molar ratio) and DODC:DOPE:cholesterol:PEG-lipid:DiVA-PEG-DiVA formulations wherein the DiVA-PEG-DiVA is co-solubilized.

Other formulations of the invention include those comprising HEDODC:DOPE: cholesterol-PEG-lipid:DiVA-PEG-DiVA (50:10:38:2:5 molar ratio) and HEDODC:DOPE:cholesterol-PEG-lipid:DiVA-PEG-DiVA formulations wherein the DiVA-PEG-DiVA is co-solubilized.

Other preferred formulations of the invention include those comprising DC-6-14:DOPE:cholesterol:DiVA-PEG-DiVA (40:30:30:5, molar ratios) and DC-6-14:DOPE:cholesterol:DiVA-PEG-DiVA, wherein the DiVA-PEG-DiVA is co-solubilized.

Also within the scope of the invention are methods of delivering a therapeutic agent to a patient. These methods comprise providing a pharmaceutical formulation including any of the foregoing compositions and a pharmaceutically acceptable carrier or diluent; and administering the pharmaceutical formulation to the patient.

### DEFINITIONS

As used herein, “alkyl” refers to a straight or branched fully saturated (no double or triple bonds) hydrocarbon group, for example, a group having the general formula —CₙH₂ₙ₊₁. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “halogen” refers to F, Cl, Br, and I.

As used herein, “mesylate” refers to —OSO₂CH₃.

As used herein, the term “pharmaceutical formulation” refers to a mixture of a composition disclosed herein with one or more other chemical components, such as diluents or additional pharmaceutical carriers. The pharmaceutical formulation facilitates administration of the composition to an organism. Multiple techniques of administering a pharmaceutical formulation exist in the art including, but not limited to injection and parenteral administration.

As used herein, the term “pharmaceutical carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism

As used herein, the term “diluent” refers to chemical compounds diluted in water that will dissolve the formulation of interest (e.g., the formulation that can include a compound, a retinoid derivative and/or vitamin A analogue, a second lipid, a stabilizing agent, and/or a therapeutic agent) as well as stabilize the biologically active form of the formulation. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of the formulation. As used herein, an “excipient” refers to an inert substance that is added to a formulation to provide, without limitation, bulk, consistency, stability, binding ability, lubrication, disintegrating ability, etc., to the composition. A “diluent” is a type of excipient.

As used herein, “therapeutic agent” refers to a compound that, upon administration to a mammal in a therapeutically effective amount, provides a therapeutic benefit to the mammal. A therapeutic agent may be referred to herein as a drug. Those skilled in the art will appreciate that the term “therapeutic agent” is not limited to drugs that have received regulatory approval. A “therapeutic agent” can be operatively associated with a compound as described herein, a retinoid derivative and/or vitamin A analogue, and/or a second lipid. For example, a second lipid as described herein can form a liposome, and the therapeutic agent can be operatively associated with the liposome, e.g., as described herein.

As used herein, “lipoplex formulations” refer to those formulations wherein the siRNA is outside of the liposome. In preferred lipoplex formulations, the siRNA is complexed to the outside of the liposome. Other preferred lipoplex formulations include those wherein the siRNA is accessible to any medium present outside of the liposome.

As used herein, “liposome formulations” refer to those formulations wherein the siRNA is encapsulated within the liposome. In preferred liposome formulations, the siRNA is inaccessible to any medium present outside of the liposome.

As used herein, the term “co-solubilized” refers to the addition of a component to the cationic lipid mixture before the empty vesicle is formed.

As used herein, the term “decorated” refers to the addition of a component after vesicle formation.

As used herein, “DC-6-14” refers to the following cationic lipid compound:

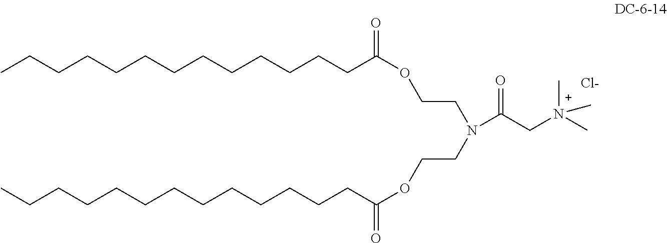 [SMILES: CCCCCCCCCCCCCC(=O)OCCN(CCOC(=O)CCCCCCCCCCCCC)C(=O)C[N+](C)(C)C ; [Cl-]]

As used herein, “DODC” refers to the following cationic lipid compound:

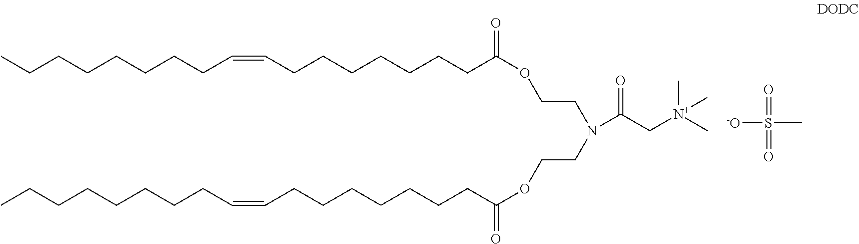

As used herein, “HEDODC” refers to the following cationic lipid compound:

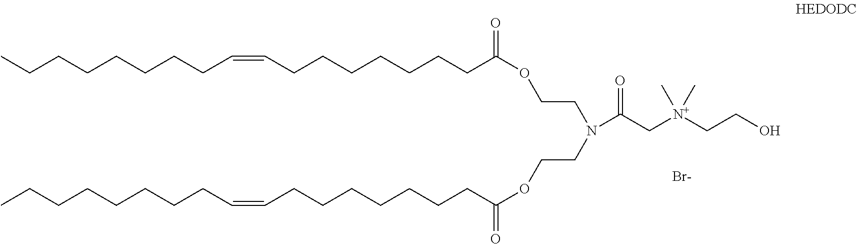 [SMILES: CCCCCCCC/C=C\CCCCCCCC(=O)OCCN(CCOC(=O)CCCCCCC/C=C\CCCCCCCC)C(=O)C[N+](C)(C)CCO ; [Br-]]

As used herein, a “retinoid” is a member of the class of compounds consisting of four isoprenoid units joined in a head-to-tail manner, see G. P. Moss, “Biochemical Nomenclature and Related Documents,” 2nd Ed. Portland Press, pp. 247-251 (1992). “Vitamin A” is the generic descriptor for retinoids exhibiting qualitatively the biological activity of retinol. As used herein, retinoid refers to natural and synthetic retinoids including first generation, second generation, and third generation retinoids. Examples of naturally occurring retinoids include, but are not limited to, (1) 11-cis-retinal, (2) all-trans retinol, (3) retinyl palmitate, (4) all-trans retinoic acid, and (5) 13-cis-retinoic acids. Furthermore, the term “retinoid” encompasses retinols, retinals, retinoic acids, rexinoids, demethylated and/or saturated retinoic acids, and derivatives thereof.

As used herein, “Vitamin D” is a generic descriptor for a group of vitamins having antirachitic activity. The vitamin D group includes: vitamin D₂ (calciferol), vitamin D₃ (irradiated 22-dihydroergosterol), vitamin D₄ (irradiated dehydrositosterol) and vitamin D₅ (irradiated dehydrositosterol).

As used herein, “Vitamin E” is a generic descriptor for a group of molecules with antioxidant activity. The vitamin E family includes α-tocopherol, β-tocopherol, γ-tocopherol and δ-tocopherol, with α-tocopherol being the most prevalent. (Brigelius-Flohe and Traber, The FASEB Journal. 1999; 13: 1145-1155).

As used herein, “Vitamin K” is generic descriptor for an antihemorrahgic factor and includes vitamin K₁ (phytonodione), vitamin K₂ (menaquinone), vitamin K₃, vitamin K₄ and vitamin K₅. Vitamins K₁ and K₂ are natural, while K3-5 are synthetic.

As used herein, “retinoid-linker-lipid molecule” refers to a molecule that includes at least one retinoid moiety attached to at least one lipid moiety through at least one linker such as, for example, a PEG moiety.

As used herein, “retinoid-linker-retinoid molecule” refers to a molecule that includes at least one retinoid moiety attached to at least one other retinoid moiety (which may be the same or different) through at least one linker such as, for example, a PEG moiety.

As used herein, the terms “lipid” and “lipophilic” are used herein in their ordinary meanings as understood by those skilled in the art. Non-limiting examples of lipids and lipophilic groups include fatty acids, sterols, C₂-C₅₀ alkyl, C₂-C₅₀ heteroalkyl, C₂-C₅₀ alkenyl, C₂-C₅₀ heteroalkenyl, C₅-C₅₀ aryl, C₅-C₅₀ heteroaryl, C₂-C₅₀ alkynyl, C₂-C₅₀ heteroalkynyl, C₂-C₅₀ carboxyalkenyl, and C₂-C₅₀ carboxyheteroalkenyl. A fatty acid is a saturated or unsaturated long-chain monocarboxylic acid that contains, for example, 12 to 24 carbon atoms A lipid is characterized as being essentially water insoluble, having a solubility in water of less than about 0.01% (weight basis). As used herein, the terms “lipid moiety” and “lipophilic moiety” refers to a lipid or portion thereof that has become attached to another group. For example, a lipid group may become attached to another compound (e.g., a monomer) by a chemical reaction between a functional group (such as a carboxylic acid group) of the lipid and an appropriate functional group of a monomer.

As used herein, “siRNA” refers to small interfering RNA, also known in the art as short interfering RNA or silencing RNA. siRNA is a class of double stranded RNA molecules that have a variety of effects known in the art, the most notable being the interference with the expression of specific genes and protein expression.

As used herein, “encapsulated by the liposome” refers to a component being substantially or entirely within the liposome structure.

As used herein, “accessible to the aqueous medium” refers to a component being able to be in contact with the aqueous medium.

As used herein, “inaccessible to the aqueous medium” refers to a component not being able to be in contact with the aqueous medium.

As used herein, “complexed on the outer surface of the liposome” refers to refers to a component being operatively associated with the outer surface of the liposome.

As used herein, “localized on the outer surface of the liposome” refers to a component being at or near the outer surface of the liposome.

As used herein, “charge complexed” refers to an electrostatic association.

As used herein, the term “operatively associated” refers to an electronic interaction between a compound as described herein, a therapeutic agent, a retinoid derivative and/or vitamin A analogue, and/or a second lipid. Such interaction may take the form of a chemical bond, including, but not limited to, a covalent bond, a polar covalent bond, an ionic bond, an electrostatic association, a coordinate covalent bond, an aromatic bond, a hydrogen bond, a dipole, or a van der Waals interaction. Those of ordinary skill in the art understand that the relative strengths of such interactions may vary widely.

The term “liposome” is used herein in its ordinary meaning as understood by those skilled in the art, and refers to a lipid bilayer structure that contains lipids attached to polar, hydrophilic groups which form a substantially closed structure in aqueous media. In some embodiments, the liposome can be operatively associated with one or more compounds, such as a therapeutic agent and a retinoid derivative and/or vitamin A analogue or retinoid conjugate. A liposome may be comprised of a single lipid bilayer (i.e., unilamellar) or it may comprised of two or more concentric lipid bilayers (i.e., multilamellar). Additionally, a liposome can be approximately spherical or ellipsoidal in shape.

The term “facilitating drug delivery to a target cell” refers the enhanced ability of the present retinoid derivative and/or vitamin A analogue or fat soluble vitamin compounds to enhance delivery of a therapeutic molecule such as siRNA to a cell. While not intending to be bound by theory, the retinoid derivative and/or vitamin A analogue or fat-soluble vitamin compound interacts with a specific receptor (or activation/binding site) on a target cell with specificity that can be measured. For example, binding is generally consider specific when binding affinity (Kₐ) of 10⁶M⁻¹ or greater, preferably 10⁷ M⁻¹ or greater, more preferably 10⁸M⁻¹ or greater, and most preferably 10⁹M⁻¹ or greater. The binding affinity of an antibody can be readily determined by one of ordinary skill in the art, for example, by Scatchard analysis (Scatchard, Ann. NY Acad. Sci. 51:660, 1949).

In another aspect, the present invention also relates to a medicine (or a pharmaceutical formulation) for treating a stellate cell-related disorder, the medicine containing the drug carrier and the drug for controlling the activity or growth of stellate cells, and relates to the use of the drug carrier in the production of a medicine for treating a stellate cell-related disorder. The stellate cell-related disorder referred to here means a disorder in which stellate cells are directly or indirectly involved in the process of the disorder, that is, the onset, exacerbation, improvement, remission, cure, etc. of the disorder, and examples thereof include hepatic disorders such as hepatitis, in particular chronic hepatitis, hepatic fibrosis, hepatic cirrhosis, and liver cancer, and pancreatic disorders such as pancreatitis, in particular chronic pancreatitis, pancreatic fibrosis, and pancreatic cancer. Furthermore, according to recent reports, since stellate cells are present in the vocal cord (e.g. Fuja T J et al., Cell Tissue Res. 2005; 322(3): 417-24), the above-mentioned disorders include disorders of the vocal cord and larynx such as vocal cord scarring, vocal cord mucosal fibrosis, and laryngeal fibrosis.

In the medicine of the present invention, the drug carrier may include a drug in its interior, be attached to the exterior of a drug-containing substance, or be mixed with a drug as long as the retinoid derivative and/or vitamin A analogue included in the drug carrier is at least partially exposed on the exterior of the preparation before it reaches the stellate cells at the latest. Therefore, depending on the route of administration or manner in which the drug is released, the medicine may be covered with an appropriate material, such as, for example, an enteric coating or a material that disintegrates over time, or may be incorporated into an appropriate drug release system. The medicine of the present invention may be administered via various types of route including oral and parenteral routes; examples thereof include, but are not limited to, oral, intravenous, intramuscular, subcutaneous, local, rectal, intraarterial, intraportal, intraventricular, transmucosal, percutaneous, intranasal, intraperitoneal, intrapulmonary, and intrauterine routes, and the medicine may be prepared in a form appropriate for each administration route. Such a form and a preparation method may employ any known form and method as appropriate (e.g. ‘Hyoujun Yakuzaigaku’ (Standard Pharmaceutics), Ed. Y. Watanabe et al., Nankodo, 2003, etc.).

Examples of forms suitable for oral administration include, but are not limited to, powder, granule, tablet, capsule, liquid, suspension, emulsion, gel, and syrup, and examples of forms suitable for parenteral administration include injections such as injectable solution, injectable suspension, injectable emulsion, and an on-site preparation type injection. The formulation for parenteral administration may be in the form of an aqueous or nonaqueous isotonic sterile solution or suspension.

The drug carrier or the medicine of the present invention may be supplied in any configuration, but from the viewpoint of storage stability, it is preferably provided in a configuration that allows on-site preparation, for example, in a configuration that allows a doctor and/or a pharmacist, a nurse, or another paramedic to prepare it at the place of medical treatment or in the vicinity thereof. In this case, the drug carrier or the medicine of the present invention is provided as one or more containers containing at least one essential component therefor, and is prepared prior to use, for example, within 24 hours, preferably within 3 hours, and more preferably immediately prior to use. When carrying out the preparation, a reagent, a solvent, preparation equipment, etc. that are normally available at a place of preparation may be used as appropriate.

The present invention therefore includes a drug carrier or medicine preparation kit containing one or more containers containing one or more of a drug carrier constituent, a retinoid derivative and/or a vitamin A analogue, and/or a drug, and also includes an essential component for the drug carrier or the medicine provided in the form of such a kit. The kit of the present invention may contain, in addition to those described above, a description, etc. in which a preparation method or an administration method for the drug carrier and the medicine of the present invention is described. Furthermore, the kit of the present invention may contain all components for completing the drug carrier or the medicine of the present invention but need not necessarily contain all of the components. The kit of the present invention therefore need not contain a reagent or a solvent that is normally available at a place of medical treatment, an experimental facility, etc. such as, for example, sterile water, saline, or a glucose solution.

In another aspect, the present disclosure relates to a pharmaceutical formulation (or a medicine) comprising one or more physiologically acceptable surface active agents, pharmaceutical carriers, diluents, excipients, and suspension agents, or a combination thereof; and a formulation (e.g., the formulation that can include a compound, a retinoid derivative and/or vitamin A analogue, a second lipid, a stabilizing agent, and/or a therapeutic agent) disclosed herein. Acceptable additional pharmaceutical carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, and the like may be provided in the pharmaceutical formulation. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical formulations or medicines described herein can be administered to a human patient per se, or in pharmaceutical formulations where they are mixed with other active ingredients, as in combination therapy, or suitable pharmaceutical carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may include, for example, parenteral delivery, including intramuscular, subcutaneous, intravenous, intramedullary injections, as well as intrathecal, direct intraventricular, intraperitoneal, intranasal, or intraocular injections. The formulation or medicine (e.g., the formulation that can include a compound, a retinoid derivative and/or vitamin A analogue, a second lipid, a stabilizing agent, and/or a therapeutic agent) can also be administered in sustained or controlled release dosage forms, including depot injections, osmotic pumps, and the like, for prolonged and/or timed, pulsed administration at a predetermined rate. Additionally, the route of administration may be local or systemic.

The pharmaceutical formulations or medicines may be manufactured in a manner that is itself known, e.g., by means of conventional mixing, dissolving, granulating, dragee-making, levigating, emulsifying, encapsulating, entrapping or tableting processes.

Pharmaceutical formulations or medicines may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, sucrose, glucose, dextrose, mannitol, lactose, lecithin, albumin, sodium glutamate, cysteine hydrochloride, and the like. In addition, if desired, the injectable pharmaceutical formulations may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations may be utilized.

Pharmaceutical formulations or medicines for parenteral administration, e.g., by bolus injection or continuous infusion, include aqueous solutions of the active formulation (e.g., the formulation that can include a compound, a retinoid derivative and/or vitamin A analogue, a second lipid, a stabilizing agent, and/or a therapeutic agent) in water-soluble form. Additionally, suspensions of the active compounds may be prepared as appropriate oily injection suspensions. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations or medicines for injection may be presented in unit dosage form, e.g., in ampoules or in multi-dose containers, with an added preservative. The formulations or medicines may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the preparations described previously, the formulations or medicines may also be formulated as a depot preparation. Such long acting formulations may be administered by intramuscular injection. Thus, for example, the formulations or medicines (e.g., the formulation that can include a compound, a retinoid derivative and/or vitamin A analogue, a second lipid, a stabilizing agent, and/or a therapeutic agent) may be formulated with suitable polymeric or hydrophobic materials (for example as an emulsion in an acceptable oil) or ion exchange resins, or as sparingly soluble derivatives, for example, as a sparingly soluble salt.

Some embodiments herein are directed to a method of delivering a therapeutic agent to a cell. For example, some embodiments are directed to a method of delivering a therapeutic agent such as siRNA into a cell. Suitable cells for use according to the methods described herein include prokaryotes, yeast, or higher eukaryotic cells, including plant and animal cells (e.g., mammalian cells). In these embodiments, the formulations described herein can be used to transfect a cell. These embodiments may include contacting the cell with a formulation described herein that includes a therapeutic agent, to thereby deliver a therapeutic agent to the cell.

The present invention further relates to a method for treating a stellate cell-related disorder, the method including administering an effective amount of the medicine or the formulations to a subject in need thereof. The effective amount referred to here is an amount that suppresses onset of the target disorder, reduces symptoms thereof, or prevents progression thereof, and is preferably an amount that prevents onset of the target disorder or cures the target disorder. It is also preferably an amount that does not cause an adverse effect that exceeds the benefit from administration. Such an amount may be determined as appropriate by an in vitro test using cultured cells, etc. or by a test in a model animal such as a mouse, a rat, a dog, or a pig, and such test methods are well known to a person skilled in the art.

The dosage of a medicine or a formulation administered by the method of the present invention depends on the type of drug used or the type of retinoid derivative and/or vitamin A analogue and, for example, when an siRNA for HSP47 is used as the drug, the weight of the drug is, for example, 0.01 to 45 mg/kg/day, preferably 0.1 to 30 mg/kg/day, more preferably 1 to 20 mg/kg/day, and most preferably 4 to 6 mg/kg/day. When vitamin A is used as the retinoid derivative and/or vitamin A analogue, vitamin A is typically administered at a dosage of 10 to 20 mg/kg/day. The retinoid derivative and/or vitamin A analogue contained in the drug carrier and the dosage of the drug used in the method of the present invention are either known to a person skilled in the art or are determined as appropriate by the above-mentioned test, etc.

A specific dosage of a medicine or the formulations administered in the method of the present invention can be determined while taking into consideration various conditions of a subject that requires treatment, for example, the severity of symptoms, general health conditions of the subject, age, weight, sex of the subject, diet, the timing and frequency of administration, a medicine used in combination, responsiveness to treatment, and compliance with treatment, and it might be different from the above-mentioned typical dosage, but in such a case, these methods are still included in the scope of the present invention.

With regard to the administration route, there are various routes including both oral and parenteral routes such as, for example, oral, intravenous, intramuscular, subcutaneous, local, rectal, intraarterial, intraportal, intraventricular, transmucosal, percutaneous, intranasal, intraperitoneal, intrapulmonary, and intrauterine routes.

The frequency of administration depends on the properties of the medicine used and the above-mentioned conditions of the subject and may be, for example, a plurality of times a day (i.e. 2, 3, 4, 5, or more times per day), once a day, every few days (i.e. every 2, 3, 4, 5, 6, or 7 days, etc.), once a week, or once every few weeks (i.e. once every 2, 3, or 4 weeks, etc.).

In the method of the present invention, the term ‘subject’ means any living individual, preferably an animal, more preferably a mammal, and yet more preferably a human individual. In the present invention, the subject may be healthy or affected with some disorder, and in the case of treatment of a disorder being intended, the subject typically means a subject affected with the disorder or having a risk of being affected.

Furthermore, the term ‘treatment’ includes all types of medically acceptable prophylactic and/or therapeutic intervention for the purpose of the cure, temporary remission, prevention, etc. of a disorder. For example, when the disorder is hepatic fibrosis, the term ‘treatment’ includes medically acceptable intervention for various purposes including delaying or halting the progression of fibrosis, regression or disappearance of lesions, prevention of the onset of fibrosis, or prevention of recurrence.

Also disclosed herein are methods for treating a condition characterized by abnormal fibrosis, which may include administering a therapeutically effective amount of a formulation or a medicine described herein. Conditions characterized by abnormal fibrosis may include a fibrotic disease. Types of fibrotic disease that may be treated or ameliorated by a formulation described herein include, but are not limited to, hepatic fibrosis, hepatic cirrhosis, pancreatitis, pancreatic fibrosis, cystic fibrosis, vocal cord scarring, vocal cord mucosal fibrosis, laryngeal fibrosis, pulmonary fibrosis, idiopathic pulmonary fibrosis, cystic fibrosis, myelofibrosis, retroperitoneal fibrosis, and nephrogenic systemic fibrosis. In an embodiment, the condition that may be treated or ameliorated is hepatic fibrosis.

The medicines, formulations or pharmaceutical compositions described herein may be administered to the subject by any suitable means. Non-limiting examples of methods of administration include, among others, (a) administration via injection, subcutaneously, intraperitoneally, intravenously, intramuscularly, intradermally, intraorbitally, intracapsularly, intraspinally, intrasternally, or the like, including infusion pump delivery; (b) administration locally such as by injection directly in the renal or cardiac area, e.g., by depot implantation; as well as deemed appropriate by those of skill in the art for bringing the active compound into contact with living tissue.

Pharmaceutical compositions (or medicines) suitable for administration include formulations (e.g., the formulation that can include a compound, a retinoid derivative and/or vitamin A analogue, a second lipid, a stabilizing agent, and/or a therapeutic agent) where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of composition effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art.

The exact formulation, route of administration and dosage for the pharmaceutical compositions or the medicines can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition or the medicine administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the dosages will be about the same, or dosages that are about 0.1% to about 500%, more preferably about 25% to about 250% of the established human dosage. Where no human dosage is established, as will be the case for newly-discovered pharmaceutical compositions or medicines, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, a dose of about 0.1 mg to 2000 mg of each active ingredient, preferably about 1 mg to about 500 mg, e.g. 5 to 200 mg. In other embodiments, an intravenous, subcutaneous, or intramuscular dose of each active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free base. In some embodiments, the formulation is administered 1 to 4 times per day. Alternatively the formulations may be administered by continuous intravenous infusion, preferably at a dose of each active ingredient up to about 1000 mg per day. As will be understood by those of skill in the art, in certain situations it may be necessary to administer the formulations or the medicines disclosed herein in amounts that exceed, or even far exceed, the above-stated, preferred dosage range in order to effectively and aggressively treat particularly aggressive diseases or infections. In some embodiments, the formulations or the medicines will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of formulation or medicine administered may be dependent on the subject being treated, on the subject's weight, the severity of the affliction, the manner of administration and the judgment of the prescribing physician.

Formulations or medicines disclosed herein (e.g., the formulation that can include a compound, a retinoid derivative and/or vitamin A analogue, a second lipid, a stabilizing agent, and/or a therapeutic agent) can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The formulations or medicines may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

The present invention also relates to a method for delivering a drug to stellate cells using the drug carrier. This method includes, but is not limited to, a step of supporting a substance to be delivered on the drug carrier, and a step of administering or adding the drug carrier carrying the substance to be delivered to a stellate cell-containing living body or medium, such as, for example, a culture medium. These steps may be achieved as appropriate in accordance with any known method, the method described in the present specification, etc. This delivery method may be combined with another delivery method, for example, another delivery method in which an organ where stellate cells are present is the target, etc.

It is understood that, in any compound described herein having one or more stereocenters, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z a mixture thereof. Likewise, all tautomeric forms are also intended to be included.

### EXAMPLES

The Examples below are only intended to explain the present invention, and the scope of the present invention is not limited by specific numeric values and procedures shown in the Examples.

### Example 1

### Preparation of siRNA for gp46

Among optimal sequences for siRNA recognition in targeting a base sequence of HSP47, which is a common molecular chaperone for collagens (types I to IV), Sequences A and B were prepared in accordance with an siRNA oligo design program by iGENE Therapeutics, Inc. Sequence C was prepared by searching on the Internet using the siRNA Target Finder (http://www.ambion.com/techlib/misc/siRNA_finder.html) from Ambion, Inc. and selecting 19 base sequences that would become a target for rat gp46 (human HSP47 homologue, GenBank Accession No. M69246). When carrying out the design, care was taken in 1) starting at 75 to 100 bases downstream from the initiation codon, 2) positioning the first AA dimer, and 3) making sure that the GC content was 30% to 70%. In this example, siRNAs having the sequences below were prepared.

A: GUUCCACCAUAAGAUGGUAGACAAC (25 base forward direction strand siRNA starting at 757th in the sequence, SEQ ID NO:5)

B: CCACAAGUUUUAUAUCCAAUCUAGC (25 base forward direction strand siRNA starting at 1626th in the sequence, SEQ ID NO:6)

C: GAAACCUGUAGAGGCCGCA (19 base forward direction strand siRNA starting at 64th in the sequence, SEQ ID NO:7)

### Example 2

### Inhibition of gp46 Expression by Prepared siRNA

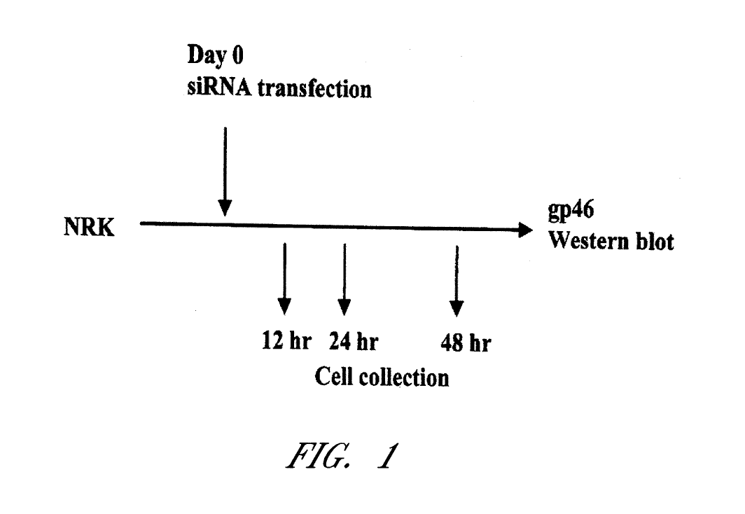

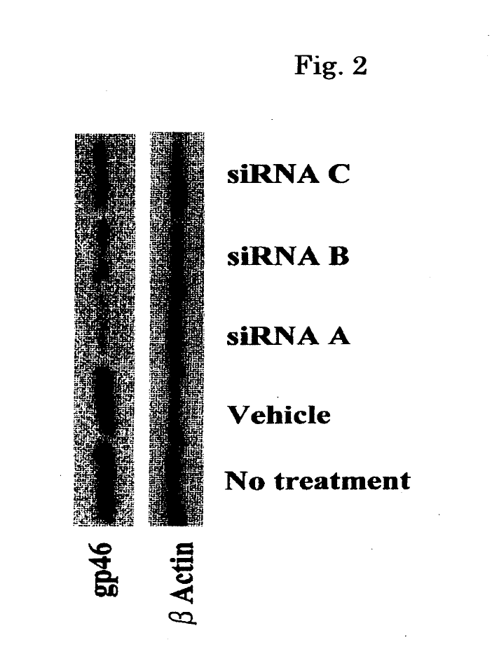

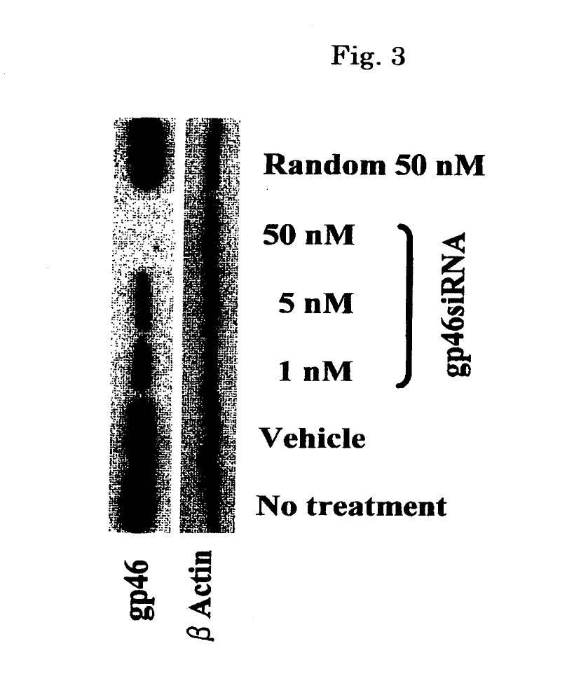

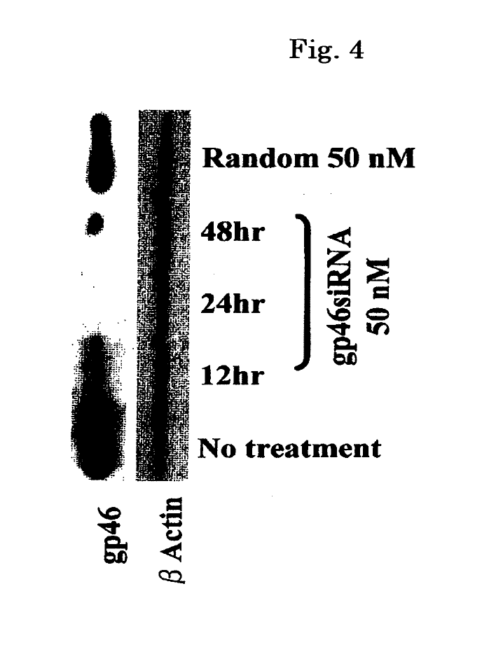

Normal rat kidney cells (NRK cells), which had rat gp46 and were fibroblasts producing collagen, were transfected with 0.1 nM to 50 nM siRNA and cultured for 12 to 48 hours (FIG. 1). The amount of expression of gp46 was checked by the western blot method (FIGS. 2 to 4, upper band corresponding to gp46, lower band corresponding to actin control). All of the siRNAs inhibited the expression of gp46 protein remarkably compared with a vehicle (FIG. 2). In the experiment below, siRNA Sequence A, which showed the strongest effect, was used. Inhibition by siRNA was concentration dependent (FIG. 3); protein expression by gp46 was about 90% inhibited by 50 nM siRNA at 48 hours (FIG. 4).

### Example 3

### Inhibition of Collagen Synthesis by Prepared siRNA

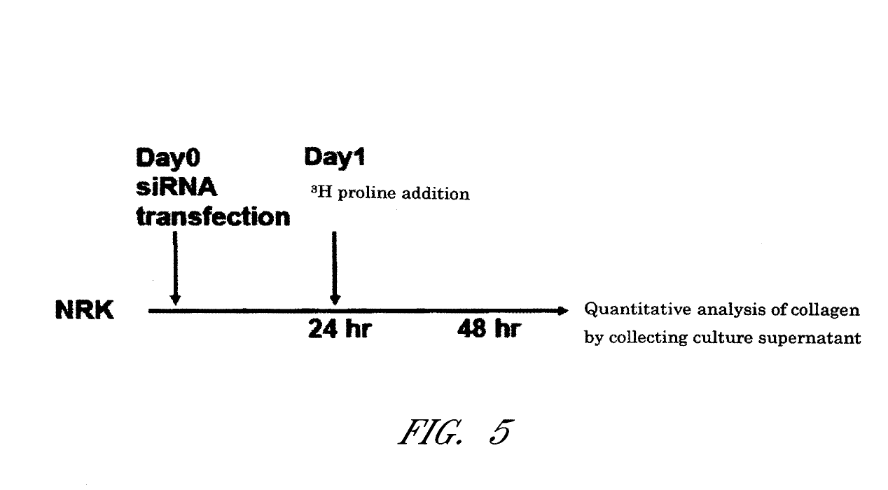

In order to examine the amount of collagen synthesized, ³H-proline was added to the culture supernatant of rat fibroblasts (NRK cells) under the above-mentioned conditions (siRNA concentration 50 nM, time 48 hours), and after transfection the amount of ³H in secreted protein was examined (FIG. 5). The amount of collagen synthesized was calculated from the ratio of protein secreted in the supernatant to protein degraded by collagenase when culturing gp46siRNA-transfected fibroblasts in the presence of ³H-proline in accordance with a report by Peterkofsky et al. (Peterkofsky et al., Biochemistry. 1971 March 16; 10(6): 988-94).

$$collagen synthesis ratio = \frac{collagenase \text{-} sensitive f raction \times 100}{( \begin{matrix}5.4 \times collagenase \text{-} insensitive fraction + \\ collagenase \text{-} sensitive fraction\end{matrix} )} \qquad ( Equation 1 )$$

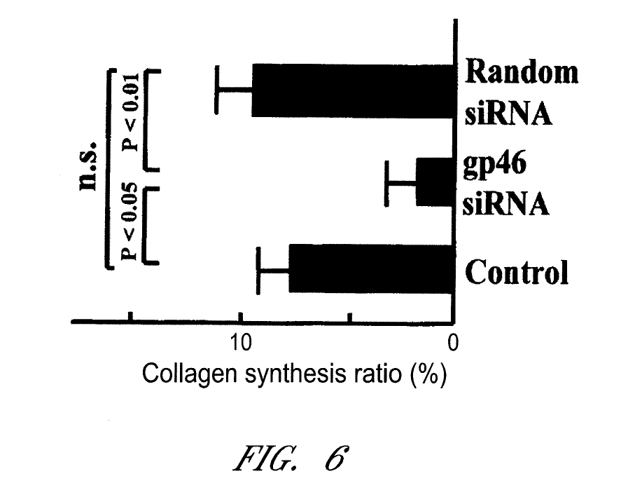

The collagen synthesis ratio in rat fibroblasts decreased by about 40% compared with a Control group (FIG. 6).

### Example 4

### Specific Transfection of Nucleic Acid into Hepatic Stellate Cells (HSC)

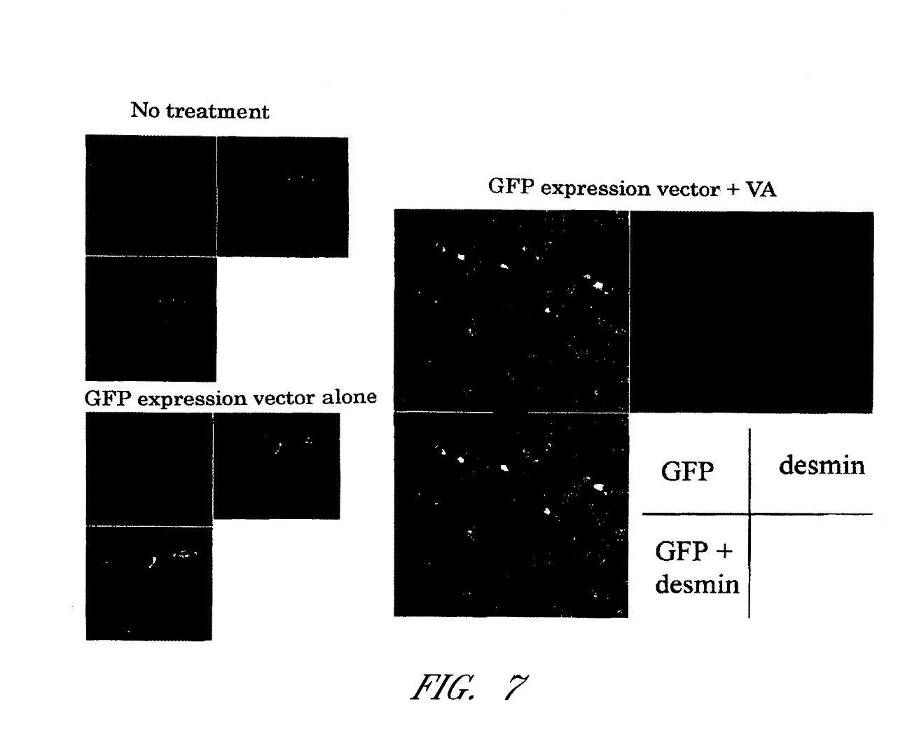

An emulsion (VA-Lip-GFP) was prepared by mixing GFP expression plasmid and liposome-encapsulated VA formed by mixing 10% VA and liposome, and after it was intraportally administered to a rat, hepatic tissue was collected and fixed. The emulsion was prepared by supposing that the amount of plasma for a 200 g rat was about 10 mL, and setting the concentrations of VA and GFP in portal blood at 10 μM. Specifically, 25 mg of all-trans-retinol (VA) was first dissolved in 87 μL of DMSO thus to give a 100 mM stock solution. 1 μL of this VA stock solution was mixed with 10 μL of lipofectamine and 179 μL of PBS, 10 μg of GFP expression plasmid was further added thereto to give a total of 200 μL, and the mixture was vortexed for 3 minutes to give VA-Lip-GFP. The abdomen of an SD rat was opened, and the VA-Lip-GFP was slowly injected into a peripheral portal vein. 48 hours after the injection, hepatic tissue was harvested. Since compared with other hepatic cells intermediate filament desmin is specifically expressed in hepatic stellate cells (HSC), when fixed hepatic tissue was stained with Alexa Fluor 568-labeled anti-desmin antibody, and a fluorescence double image with GFP was examined, it was confirmed that GFP was expressed within the hepatic stellate cells (HSC) (FIG. 7). For untreated controls and a group to which the GFP expression plasmid vector alone was administered, expression in rat hepatic stellate cells was not observed, but in a group to which VA-Lip-GFP was administered, expression of GFP was observed specifically in stellate cells.

### Example 5

### Quantitative Analysis of Nucleic Acid Transfection Rate

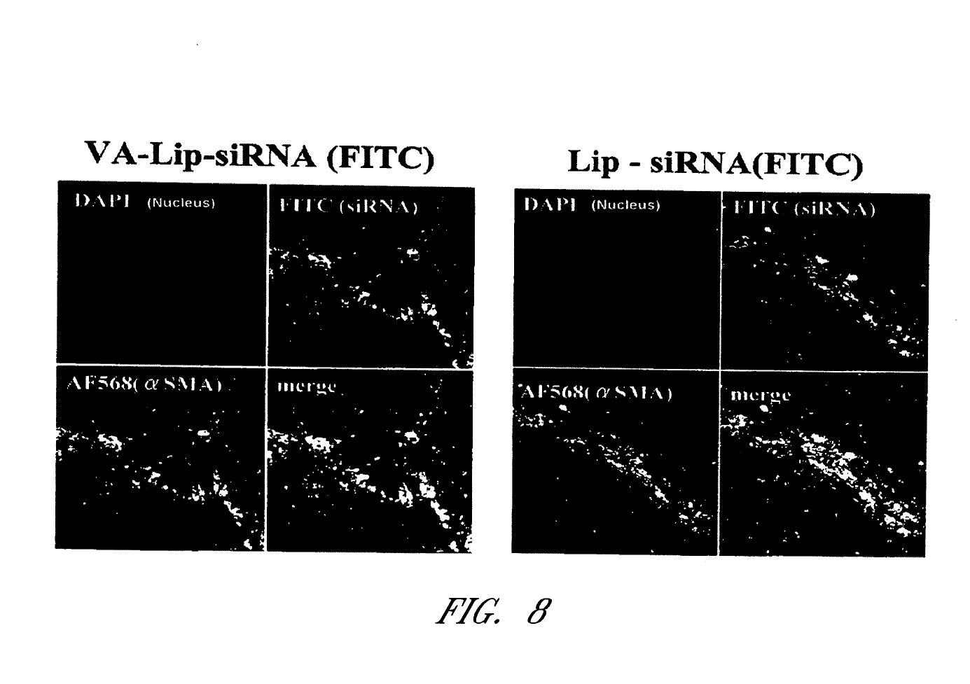

In the same manner as in Example 4, except that FITC-labeled gp46siRNA was used instead of the GFP expression plasmid, an emulsion (VA-Lip-gp46siRNA (FITC)) containing VA-encapsulated liposome and FITC-labeled gp46siRNA was prepared, and intraportally administered to an SD rat (10 μg as the amount of siRNA/200 μL). 48 hours after administration hepatic tissue was harvested, aSMA (smooth muscle actin), which compared with other hepatic cells is expressed specifically in HSC, was stained with Alexa Fluor 568-labeled anti-aSMA antibody, cell nuclei were stained with DAPI, and a fluorescence image was examined by a confocal laser scanning microscope (LSM). As shown on the left-hand side of FIG. 8, in a group to which VA-Lip-gp46siRNA (FITC) was administered, a large number of cells emitting both green fluorescence due to FITC and red fluorescence due to Alexa Fluor 568 were observed, and when a quantitative analysis was carried out by NIH Image (the number of cells was counted by selecting any 10 fields from a ×1000 fluorescence microscope photograph), the transfection efficiency was 77.6% (average of 10 fields). On the other hand, in a group to which Lip-gp46siRNA (FITC) containing no VA was administered, the transfection efficiency was a low value of 14.0% and, moreover, transfection into cells other than stellate cells was observed at 3.0% (right-hand side of FIG. 8). It has been found from the results above that the transfection efficiency into stellate cells is increased remarkably by including VA.

### Example 6

### Inhibition of Expression of gp46 by VA-Lip-gp46siRNA

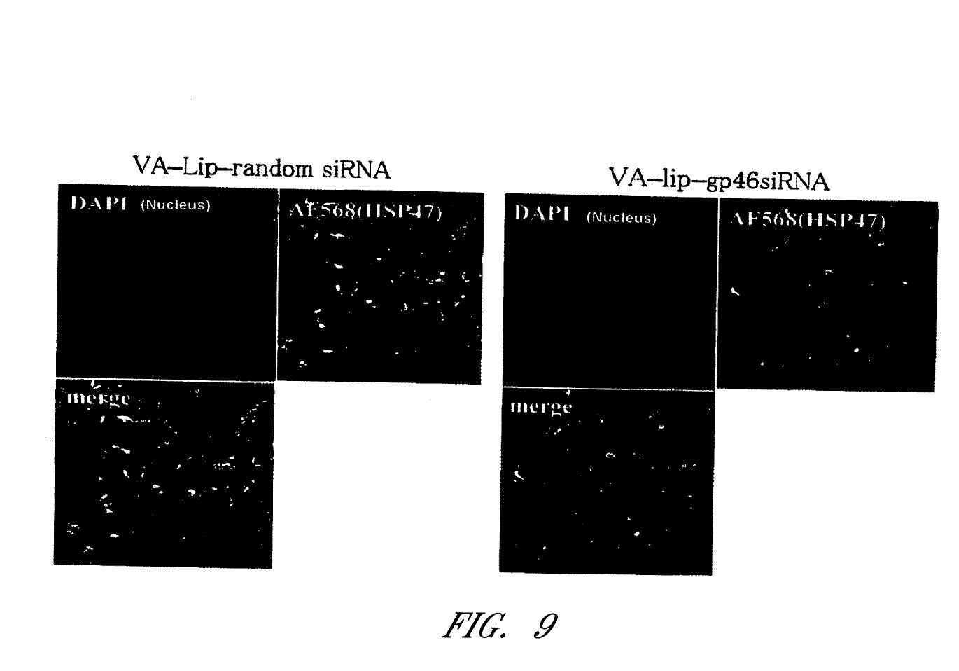

With regard to another section of the tissue harvested in Example 5, gp46 was stained with Alexa Fluor 568-labeled anti-HSP47 antibody and cell nuclei were stained with DAPI, and a fluorescence image was examined by a confocal laser scanning microscope. As shown in FIG. 9, it was observed that in a group to which VA-Lip-gp46siRNA was administered, expression of gp46, which can be observed as a red fluorescence (right-hand side in the figure), was markedly reduced compared with a control group to which was administered VA-Lip-random siRNA containing random siRNA, which was not specific to gp46 (left-hand side in the figure). The expression inhibition rate relative to an average of 6 fields of the control group was 75%, which was extremely high, when the number of gp46-negative cells was examined by selecting any 10 fields from a ×1000 fluorescence microscope photograph using NIH Image in the same manner as in Example 7.

### Example 7

### Treatment of LC Rat (Intraportal Administration 1)

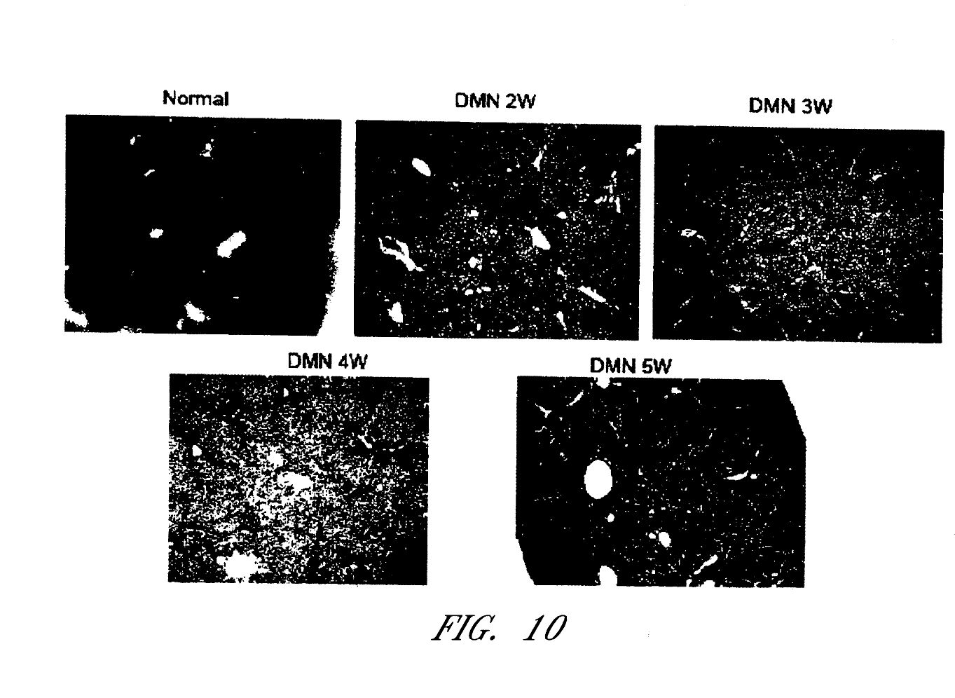

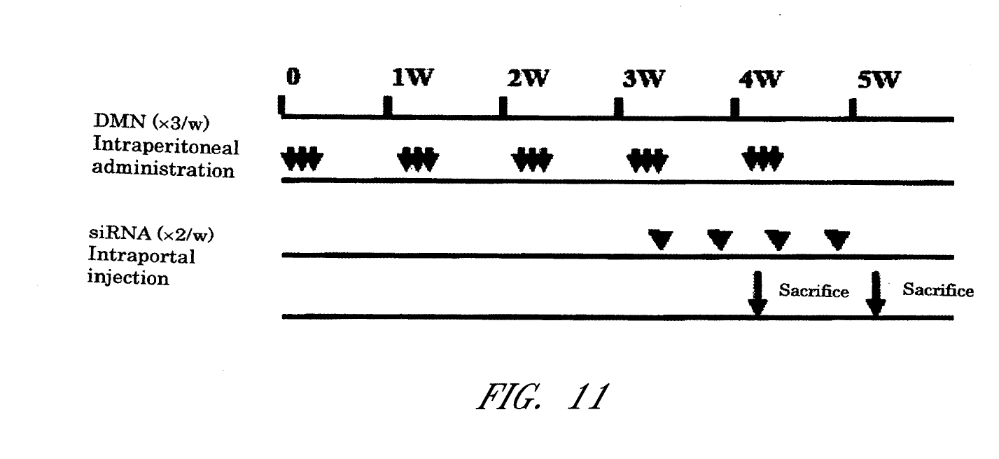

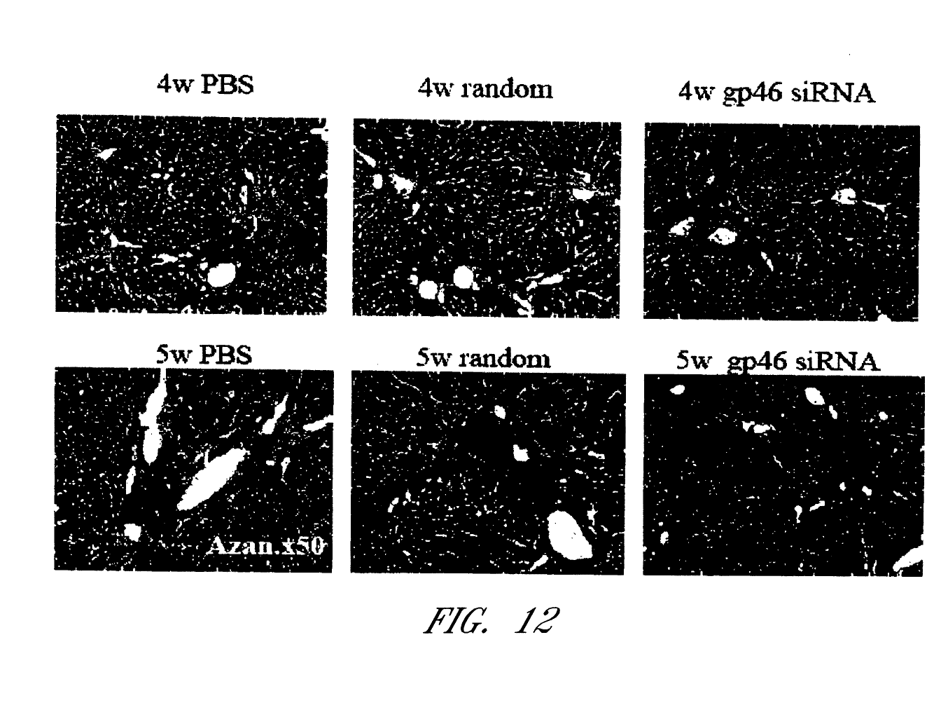

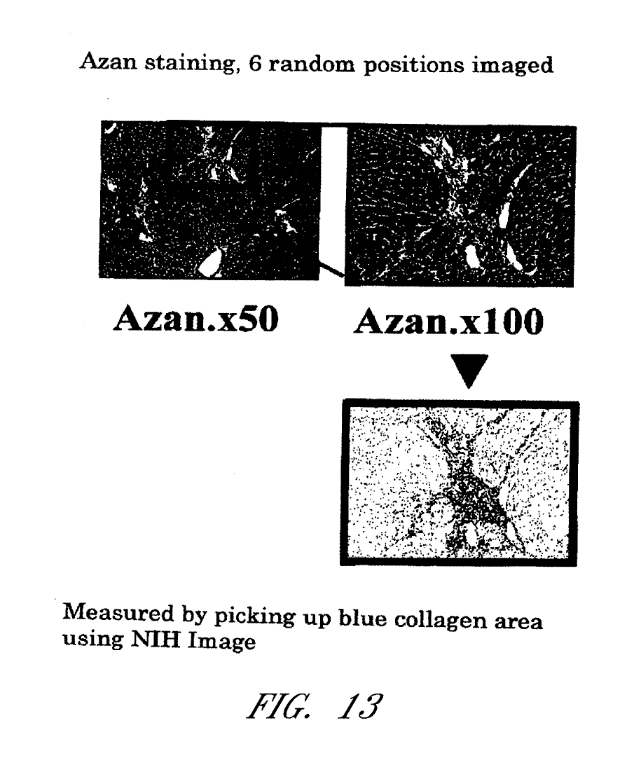

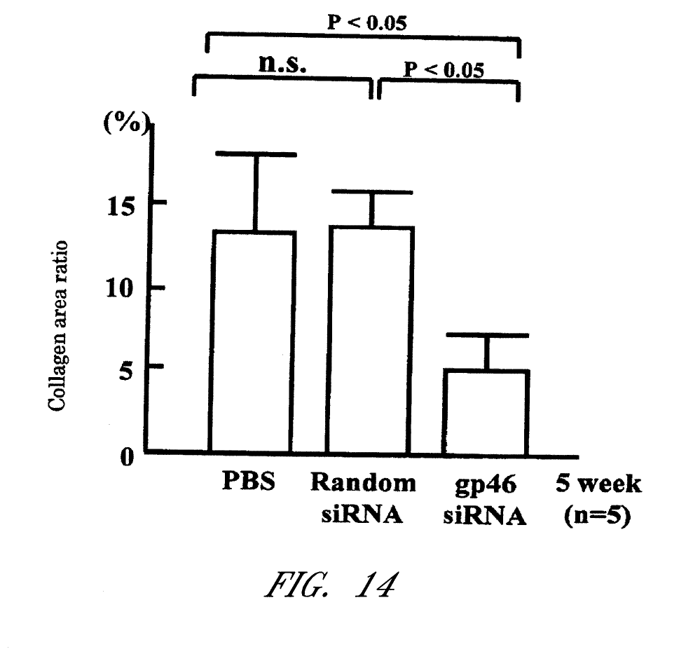

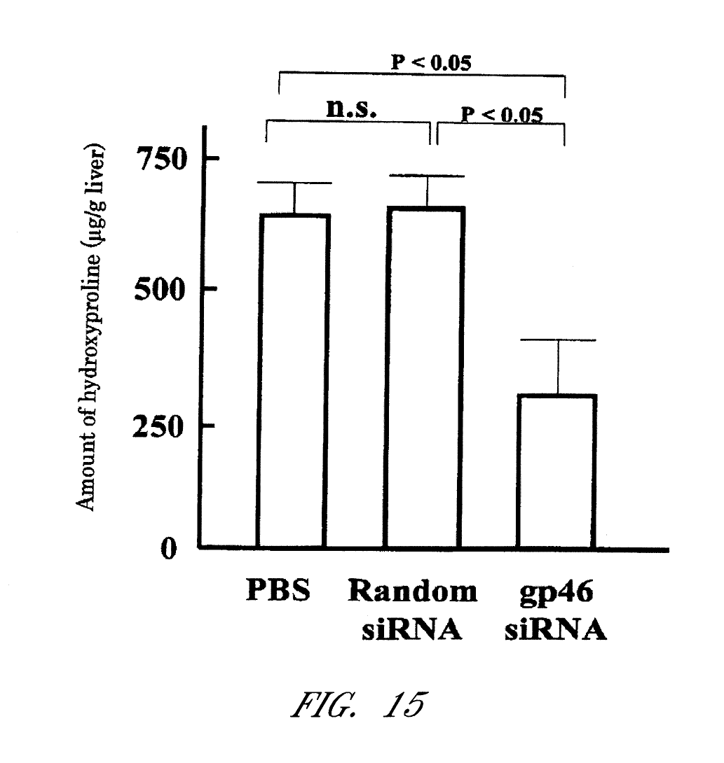

In accordance with a report by Jezequel et al. (Jezequel A M et al., J Hepatol. 1987 October; 5(2): 174-81), an LC model rat was prepared using Dimethylnitrosamine (DMN) (FIG. 10). Specifically, a 1 mL/kg dose of 1% Dimethylnitrosamine (DMN) (intraperitoneal administration) was administered to a 5 week-old SD rat (male) 3 straight days per week. As already reported, an increase in fiber was observed from the 2nd week, and in the 4th week this was accompanied by the findings of marked fibrosis, destruction of hepatic lobule structure, and formation of regenerative nodules being observed (FIG. 11). Then, by the same method as in Example 4, an emulsion (VA-Lip-gp46siRNA) was prepared by formulating gp46siRNA as a liposome and mixing with 10% VA, and was administered. Administration of VA-Lip-gp46siRNA was started in the 3rd week, by which time sufficient fibrosis was observed, and evaluation was carried out in the 4th and 5th weeks. Since it was confirmed by Example 2 that the effects were observed for up to 48 hours in vitro, administration was carried out twice a week (FIG. 11). The amount administered was determined in accordance with a report in which siRNA was directly injected (McCaffery et al., Nature. 2002 July 4; 418(6893): 38-9), and was 40 μg as the total amount of siRNA. From azan staining of the liver after administration of siRNA, in the 4th week there was no apparent difference between a group to which saline had been administered, a group to which siRNA (random) had been administered, and a group to which siRNA (gp46) had been administered, but in the 5th week a decrease in the amount of fiber was observed for the group to which gp46siRNA had been administered (FIG. 12). In order to quantitatively analyze the amount of fiber, an unstained portion was extracted using NIH Image, its area was measured (FIG. 13), and a significant decrease in the area of collagen was observed for the group to which gp46siRNA had been administered (FIG. 14). Furthermore, in order to evaluate the degree of fibrosis using another measure, the amount of hydroxyproline, which is an indicator for fibrosis, was quantitatively measured by a standard method. Specifically, after 20 mg of freeze-dried hepatic tissue was hydrolyzed with HCl for 24 hours, the reaction liquid was centrifuged, and the supernatant was treated with a reagent such as Ehrlich's solution and centrifuged. The supernatant was recovered, and the amount of hydroxyproline in the hepatic tissue was measured by measuring the absorbance at 560 nm (Hepatology 1998 November; vol. 28: 1247-1252). As shown in FIG. 15, in the group to which gp46siRNA had been administered, the amount of hydroxyproline became very small.

### Example 8

### Treatment of LC Rat (Intraportal Administration 2)

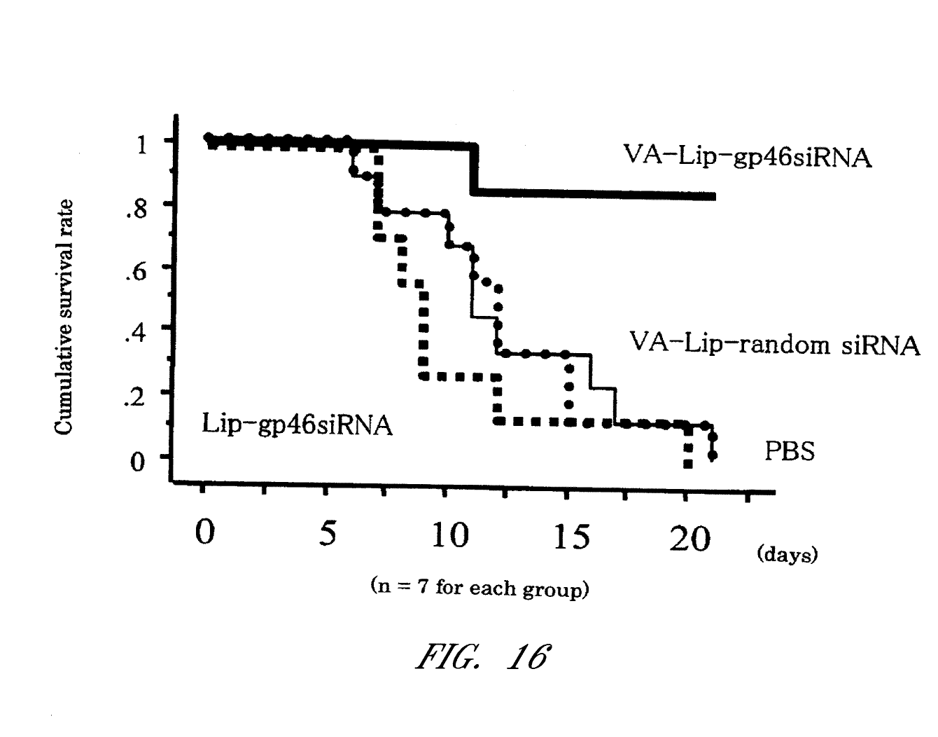

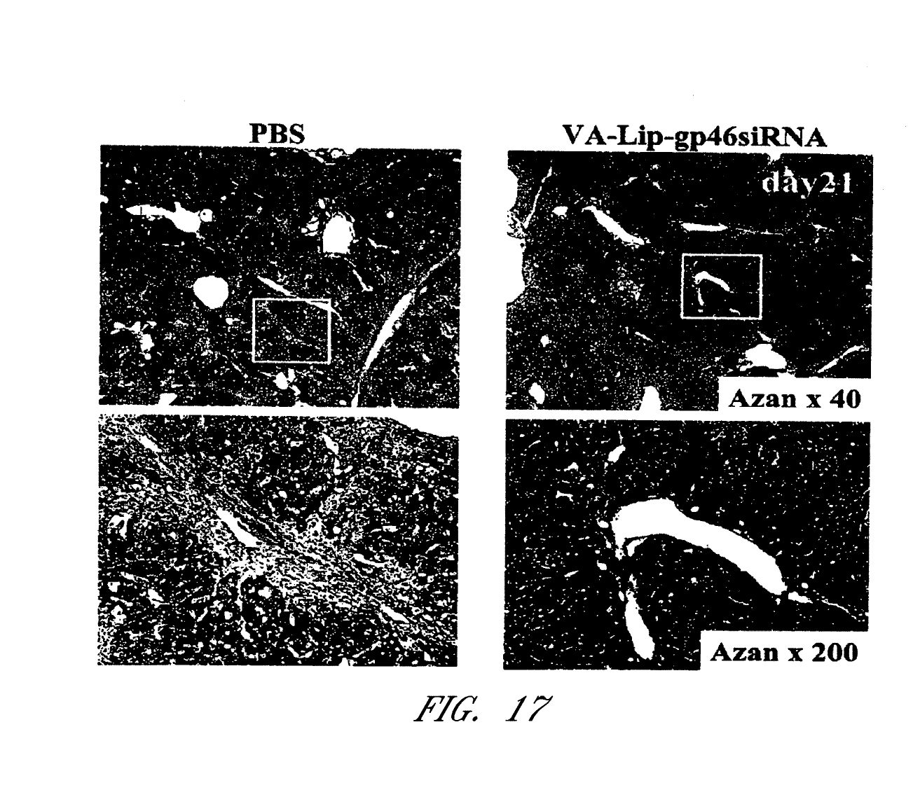

Furthermore, in order to examine a change in the survival rate by administration of the medicine of the present invention, in accordance with a method by Qi Z et al. (Proc Natl Acad Sci USA. 1999 March 2; 96(5): 2345-9), an LC model rat was prepared using Dimethylnitrosamine (DMN) in an amount that was increased by 20% over the normal amount. In this model, a total of 4 intraportal administrations were carried out in the 1st and 2nd weeks. Administration details were: PBS, Lip-gp46siRNA, VA-Lip-random siRNA, and VA-Lip-gp46siRNA (n=7 for each group). After the 3rd week, all of the controls (the group to which PBS had been administered, the group to which VA-Lip-random siRNA had been administered, and the group to which Lip-gp46siRNA had been administered) were dead, but 6 out of 7 survived for the group to which VA-Lip-gp46siRNA had been administered (FIG. 16). Furthermore, in azan staining of the liver on the 21st day, an apparent decrease in the amount of fiber was observed for the group to which gp46siRNA had been administered (FIG. 17).

### Example 9

### Treatment of LC Rat (Intraportal Administration 3)

In another experiment, intraportal administration was carried out from the 3rd week for LC model rats (1% DMN 1 mg/kg intraperitoneally administered 3 times a week) prepared in accordance with the method by Qi Z et al. and a method by Ueki T et al. (Nat Med. 1999 February; 5(2): 226-30), as shown in the table below (n=6 for each group). PBS was added to each substance to be administered so as to make a total volume of 200 μL, and the frequency of administration was once a week.

TABLE 2

| Treatment | Content of | |
|---|---|---|
| group | administration | Dosage |
| 9-1 | VA | VA 200 nmol |
| 9-2 | Lip-gp46siRNA | liposome 100 nmol, gp46siRNA 20 μg |
| 9-3 | VA-Lip-random siRNA | VA 200 nmol, liposome 100 nmol, |
| | | random-siRNA 20 μg |
| 9-4 | VA-Lip-gp46siRNA | VA 200 nmol, liposome 100 nmol, |
| | | gp46siRNA 20 μg |

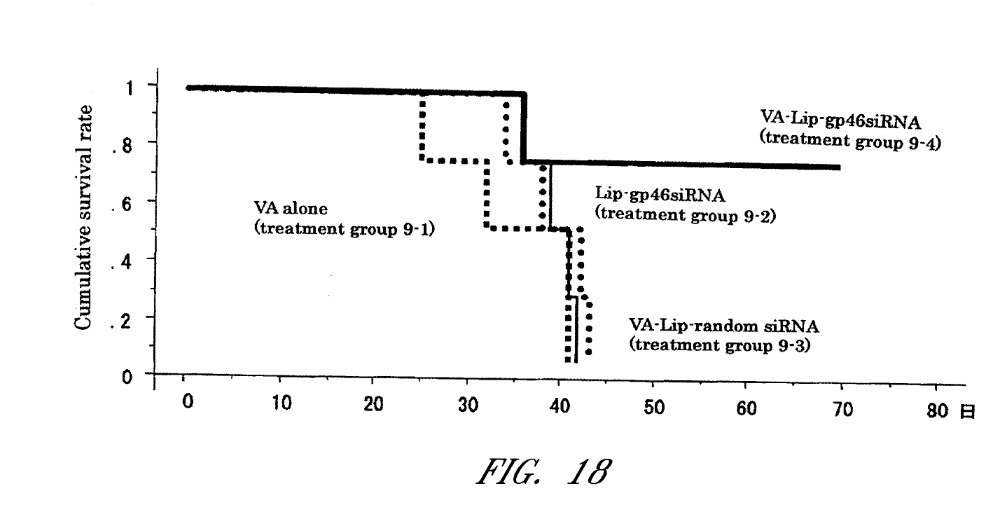

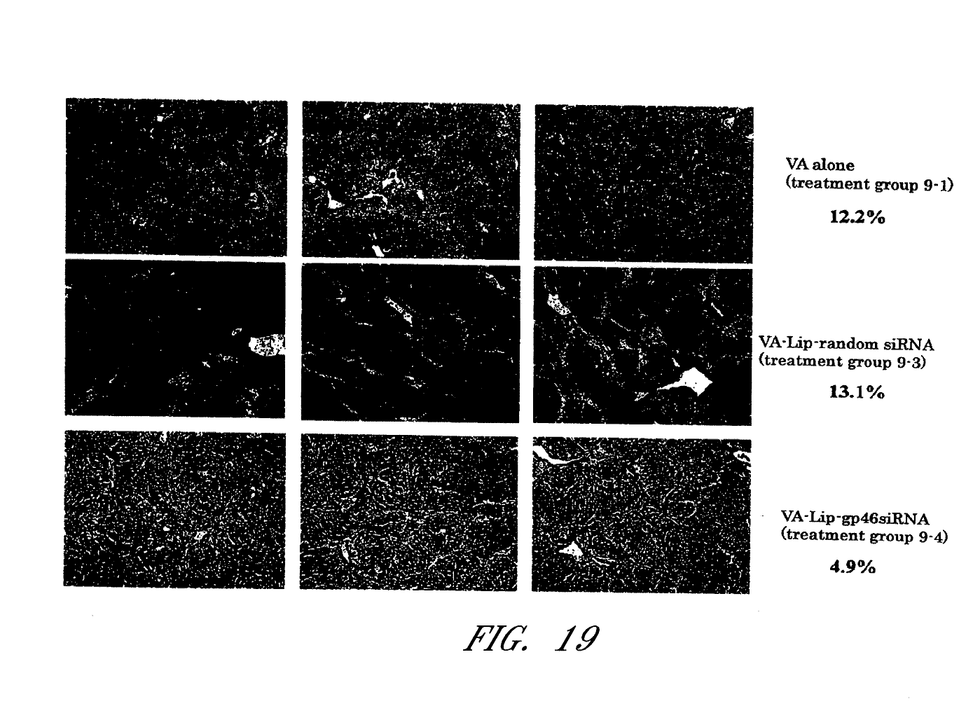

From the results, in the groups other than the group to which the medicine of the present invention had been administered (treatment group 9-4), all 6 rats were dead by the 45th day after starting administration of DMN, but in the group to which the medicine of the present invention had been administered, all of the individuals apart from one case, which was dead on the 36th day, survived for more than 70 days after starting administration of DMN (FIG. 18). For the dead individuals, the amount of hepatic fiber was quantitatively analyzed based on the area of collagen in the same manner as in Example 7, and the increase in the amount of hepatic fiber was remarkably inhibited by administration of VA-Lip-gp46siRNA (FIG. 19).

### Example 10

### Treatment of LC Rat (Intravenous Administration)

Intravenous administration was carried out from the 3rd week for LC model rats (1% DMN 1 μg/BW (g) intraperitoneally administered 3 times a week) prepared in the same manner as in Example 9, as shown in the table below (n=6 for each group). PBS was added to each substance to be administered so as to make a total volume of 200 μL. The administration period was up to death except that it was up to the 7th week for Group 10-4 and the 6th week for Group 10-10.

TABLE 3

| Treatment | Content of | | Frequency of |
|---|---|---|---|
| group | administration | Dosage | administration |
| 10-1 | VA | VA 200 nmol | Twice a week |
| 10-2 | Lip-gp46siRNA | liposome 100 nmol, | |
| | | gp46siRNA 100 μg | |
| 10-3 | VA-Lip-random | VA 200 nmol, liposome 100 | |
| | siRNA | nmol, random-siRNA 100 μg | |
| 10-4 | VA-Lip- | VA 200 nmol, liposome 100 | |
| | gp46siRNA | nmol, gp46siRNA 100 μg | |
| 10-5 | PBS | 200 μL | Three times a |
| 10-6 | VA | VA 200 nmol | week |
| 10-7 | VA-Lip | VA 200 nmol, liposome 100 | |
| | | nmol | |
| 10-8 | Lip-gp46siRNA | liposome 100 nmol, | |
| | | gp46siRNA 150 μg | |
| 10-9 | VA-Lip-random | VA 200 nmol, liposome 100 | |
| | siRNA | nmol, random-siRNA 150 μg | |
| 10-10 | VA-Lip- | VA 200 nmol, liposome 100 | |
| | gp46siRNA | nmol, gp46siRNA 150 μg | |

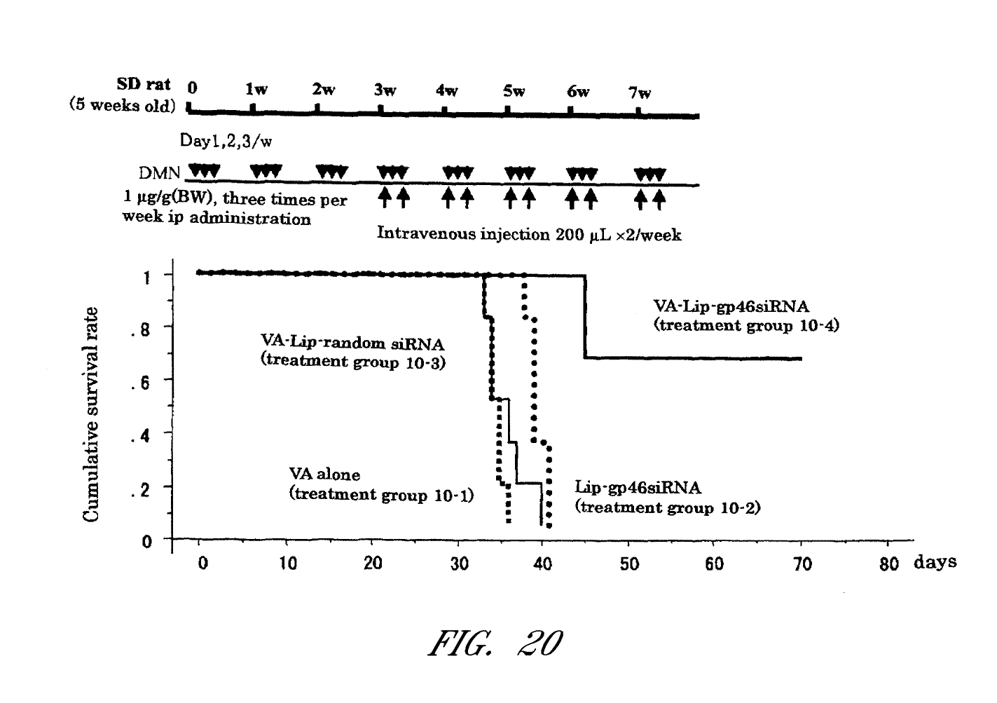

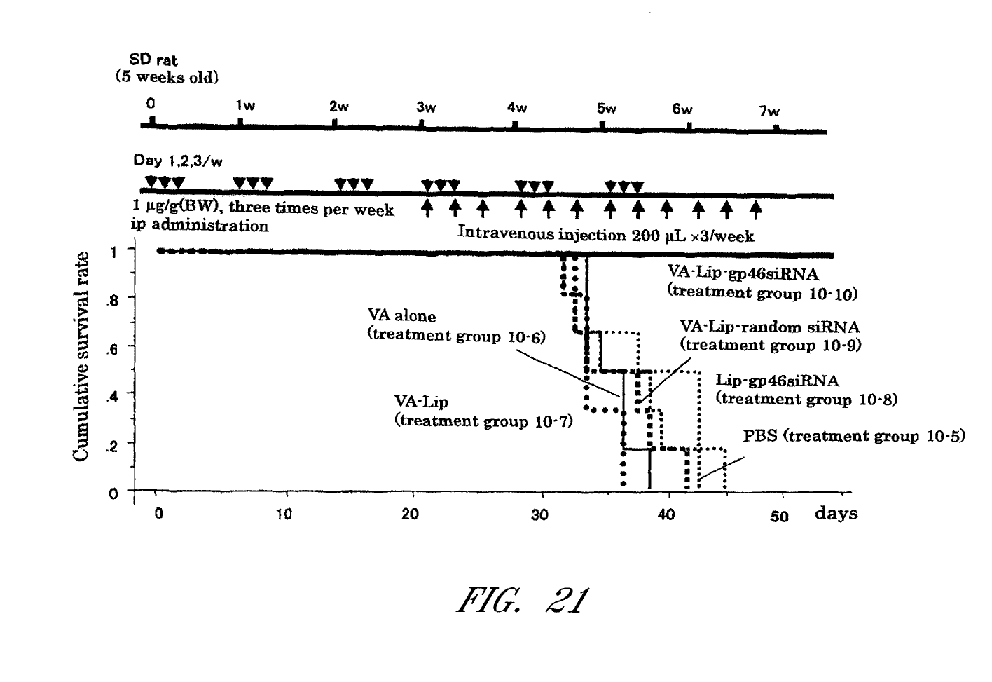

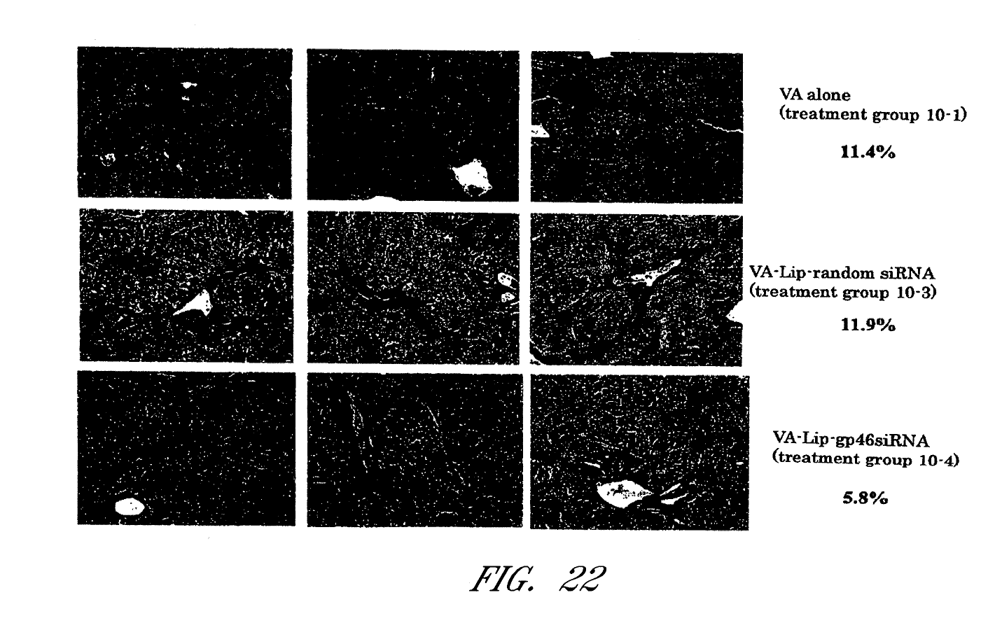

From the results, in the groups other than the groups to which the medicine of the present invention had been administered (treatment groups 10-4 and 10-10), all 6 rats were dead by the 45th day after starting administration of DMN, but in the groups to which the medicine of the present invention had been administered, all of the individuals, apart from a case in which two rats were dead on the 45th day in treatment group 10-4, survived for more than 70 days after starting administration of DMN (FIGS. 20 and 21). For the dead individuals, the amount of hepatic fiber was quantitatively analyzed in the same manner as in Example 7, and the increase in the amount of hepatic fiber was remarkably inhibited by administration of VA-Lip-gp46siRNA (FIG. 22).

The above-mentioned results show that the medicine of the present invention is extremely effective for the prevention and treatment of fibrosis, in which stellate cells are involved.

### Example 11

### Improvement of Results by RBP (Retinol-Binding Protein)

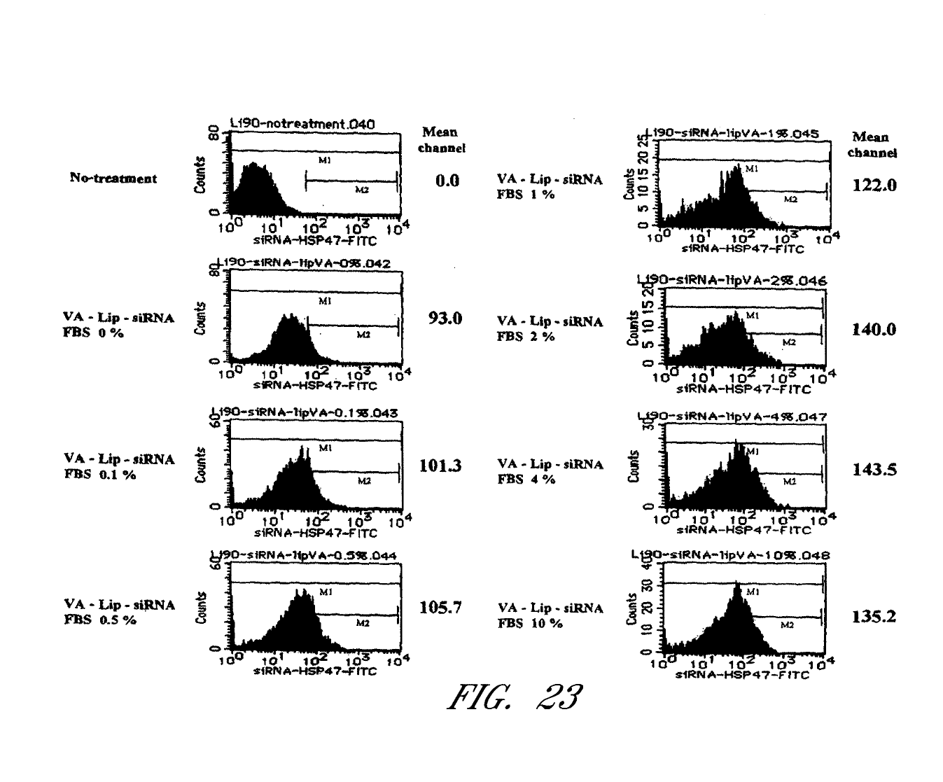

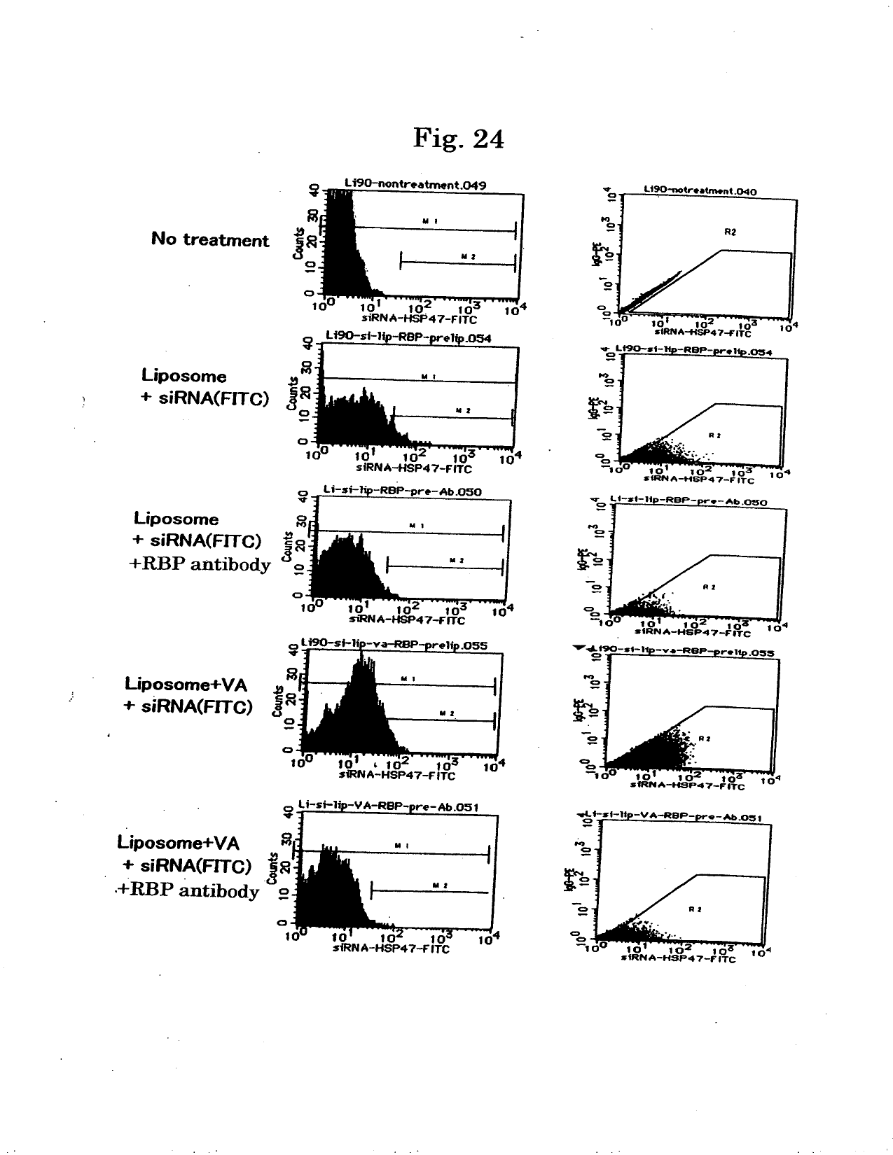

The influence of RBP on VA-Lip-gp46siRNA transfection efficiency was examined using LI90, which is a cell line derived from human hepatic stellate cells. 100 nM of VA-Lip-gp46siRNA (FITC) prepared in Example 5, together with various concentrations (i.e. 0, 0.1, 0.5, 1, 2, 4, or 10%) of FBS (fetal bovine serum), were added to LI90 during culturing and incubated for 48 hours, a fluorescence image was observed by LSM, and the amount of siRNA incorporated into individual cells was quantitatively analyzed by FACS. FBS contained about 0.7 mg/dL of RBP. As shown in FIG. 23, FBS (RBP) gave a concentration-dependent increase in the amount of siRNA transfection. Subsequently, 100 nM of VA-Lip-gp46siRNA (FITC) and 4% FBS, together with 10 μg (21.476 nmol) of anti-RBP antibody, were added to LI90 during culturing, and the siRNA transfection efficiency was evaluated in the same manner. As shown in FIG. 24, the increase in the amount of transfection by RBP was markedly decreased by the addition of anti-RBP antibody. The above-mentioned results show that RBP is effective in further enhancing transfection of the medicine of the present invention.

### Example 12

### Synthesis of DOPE-Glu-VA

### Preparation of (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate (DOPE-Glu-VA)

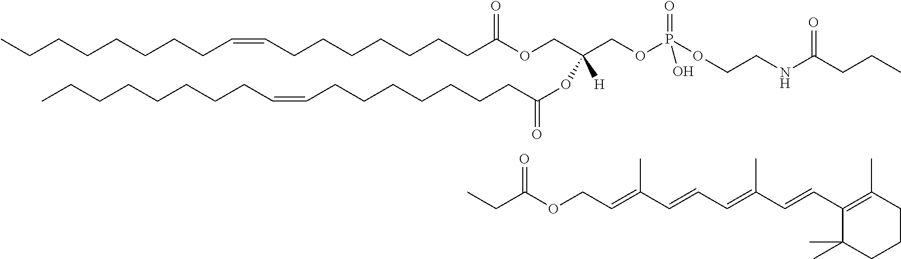

### Preparation of Intermediate 1

### 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

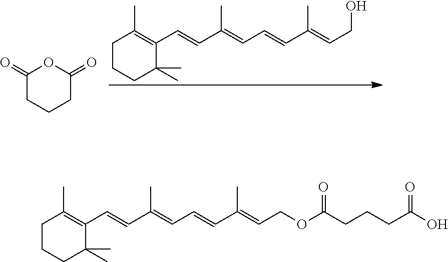

Glutaric anhydride (220 mg, 1.93 mmol) and retinol (500 mg, 1.75 mmol) were dissolved in dichloromethane (5 mL) in an amber-colored vial. Triethylamine (513 ul, 3.68 mmol) was added and the vial was flushed with argon. Reaction mixture was allowed to stir at room temperature for 4 hours. The material was concentrated and purified by silica gel chromatography with a dichloromethane/methanol gradient. Fractions were pooled and concentrated to yield yellowish oil (700 mg). The product was verified by NMR.

### Preparation of DOPE-Glu-VA

### (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate

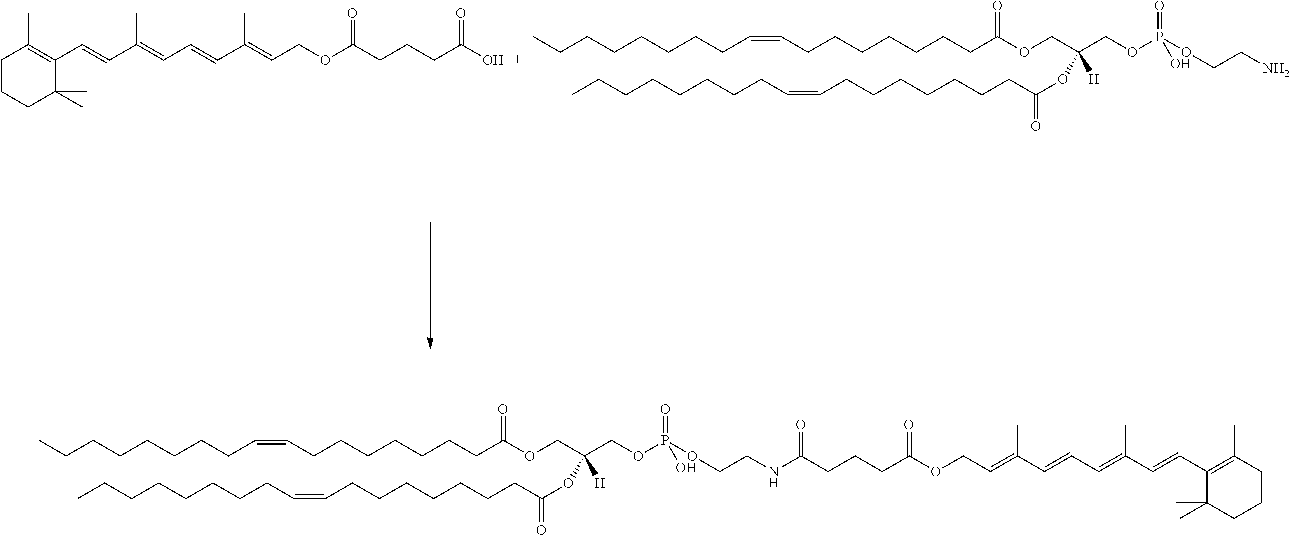

1,2-dioleoyl-sn-glycero-3-phosphoethanolamine (500 mg, 0.672 mmol), N,N,N′,N′-Tetramethyl-O-(7-azabenzotriazol-1-yl)uronium hexafluorophosphate (306.5 mg, 0.806 mmol) and 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (269 mg, 0.672 mmol) was dissolved in chloroform/DMF (10 mL, 1:1 mixture) in an amber-colored vial flushed with argon and N,N-Diisopropylethylamine (300 μL, 1.68 mmol) was added. Reaction mixture was allowed to stir overnight at room temperature. The reaction mixture was concentrated and then purified by silica gel chromatography using a dichloromethane/methanol gradient. The fractions were pooled and concentrated to yield yellowish oil (460 mg, 61%). Verified product by NMR. ¹H NMR (400 MHz), δ_{H}: 8.6 (d, 1H), 8.27 (d, 1H), 6.57-6.61 (dd, 1H), 6.08-6.25 (m, 4H), 5.57 (t, 1H), 5.30-5.34 (m, 4H), 5.18 (m, 1H), 4.68-4.70 (d, 2H), 4.28-4.35 (m, 1H), 4.05-4.15 (m, 1H), 3.81-3.97 (m, 4H), 3.52-3.62 (m, 1H), 3.35-3.45 (m, 2H), 2.95-3.05 (m, 1H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H).

### Example 13

### DOPE-Glu-NH-VA

### Preparation of (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)-phosphoryl)oxy)propane-1,2-diyl dioleate (DOPE-Glu-NH-VA)

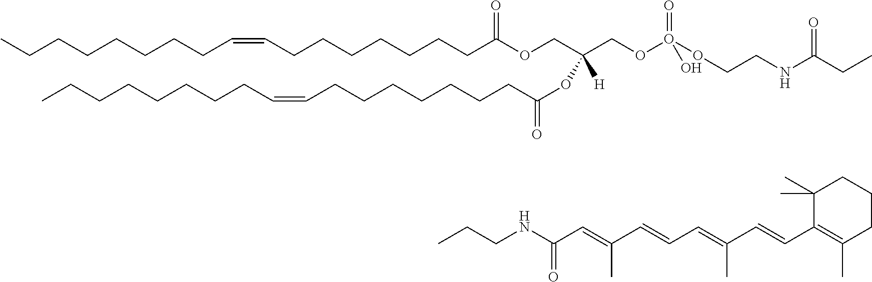

### Preparation of Intermediate 1

### (Z)-(2R)-3-(((2-(4-aminobutanamido)ethoxy)(hydroxy)phosphoryl)oxy)propane-1,2-diyl dioleate

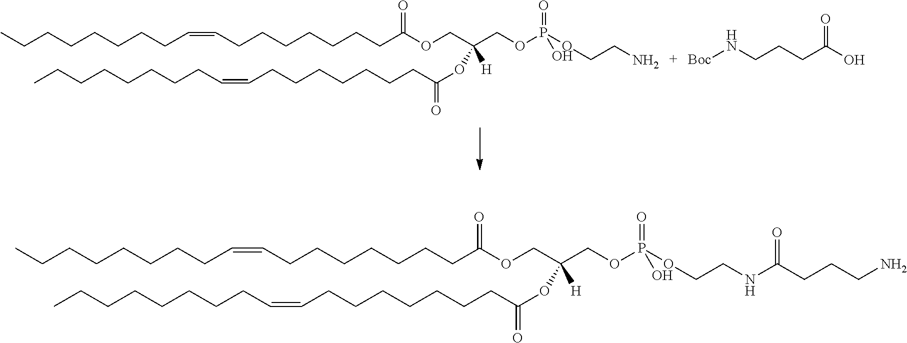

1,2-Dioleoyl-sn-glycero-3-phosphoethanolamine (2500 mg, 3.36 mmol), Boc-GABA-OH (751 mg, 3.70 mmol) and N,N,N′,N′-Tetramethyl-O-(7-azabenzotriazol-1-yl)uronium hexafluorophosphate (1531 mg, 4.03 mmol) were dissolved in a DMF/chloroform (25 mL, 1:1 mixture). N,N-Diisopropylethylamine (880 μL, 5.05 mmol) was added and the mixture was allowed to stir at room temperature overnight under a blanket of argon. The reaction mixture was diluted with ˜200 mL H₂O and product was extracted with dichloromethane (3×100 ml). The product was washed with ˜75 mL pH 4.0 PBS buffer, dried organics with sodium sulfate, filtered and concentrated. Material was then purified via silica gel chromatography with a dichloromethane/methanol gradient, and concentrated to yield colorless oil (2.01 g, 64%). The product was verified by NMR. Material was then taken up in 30 mL of 2 M HCl/diethyl ether. Reaction was allowed to stir at room temperature in a H₂O bath. After 2 hours, the solution was concentrated to yield (Z)-(2R)-3-(((2-(4-aminobutanamido)ethoxy)(hydroxy)phosphoryl)oxy)propane-1,2-diyl dioleate.

### Preparation of DOPE-Glu-NH-VA

### (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate

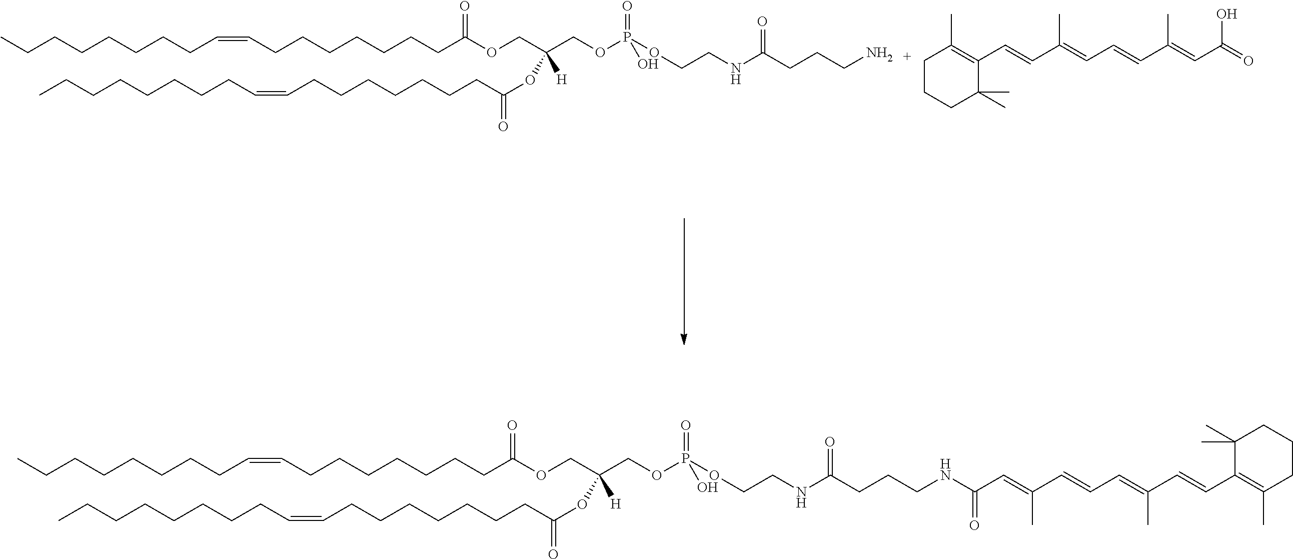

(Z)-(2R)-3-(((2-(4-aminobutanamido)ethoxy)(hydroxy)phosphoryl)-oxy)propane-1,2-diyl dioleate (1200 mg, 1.45 mmol), retinoic acid (500 mg, 1.66 mmol) and N,N,N′,N′-Tetramethyl-O-(7-azabenzotriazol-1-yl)uronium hexafluorophosphate (689 mg, 1.81 mmol) was suspended in DMF/chloroform (10 mL, 1:1 mixture). N,N-Diisopropylethylamine (758 μL, 4.35 mmol) was added. The round bottom flask was flushed with argon and covered with aluminum foil. Reaction mixture was stirred at room temperature for 4 hours, partitioned in dichloromethane (75 mL) and H₂O (75 mL), extracted with dichloromethane, dried (sodium sulfate), filtered and concentrated. Purification by silica gel chromatography using a dichloromethane/methanol gradient yielded (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (292 mg, 18%). The product was characterized by LCMS & NMR. ¹H NMR (400 MHz), δ_{H}: 8.55 (s, 1H), 8.2 (d, 1H), 7.3 (s, 1H), 6.6 (dd, 1H), 6.10-6.27 (m, 5H), 5.5 (t, 1H), 5.31 (s, 4H), 5.1-5.2 (m, 2H), 4.68 (d, 2H), 4.3 (d, 2H), 4.1 (m, 2H), 3.9 (m, 8H), 3.58 (q, 4H), 3.4 (s, 4H), 3.0 (q, 4H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H). MS: m/z 1112.44 (M+H⁺).

### Example 14

### DSPE-PEG550-VA

### Preparation of (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate (DSPE-PEG550-VA)

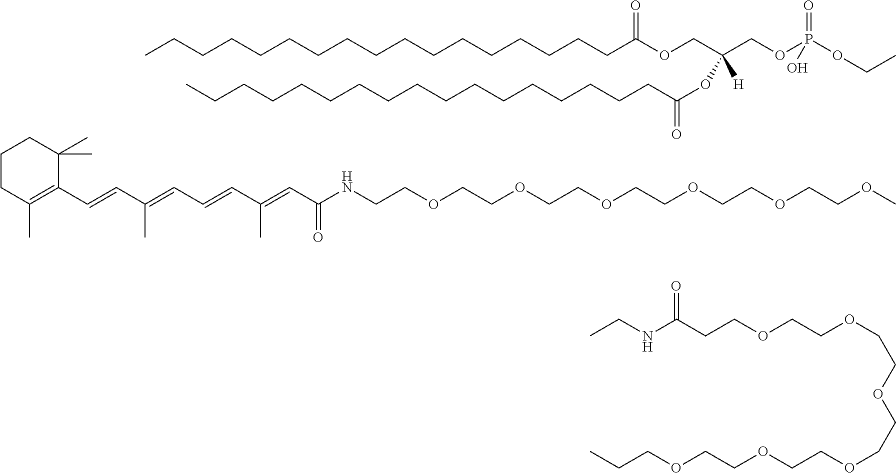

### Preparation of Intermediate 1

### (2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate

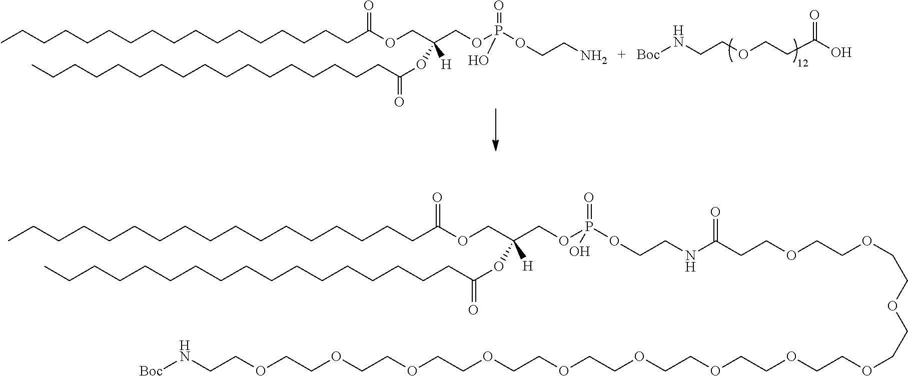

1,2-Distearoyl-sn-glycero-3-phosphoethanolamine (200 mg, 0.267 mmol), t-Boc-N-amido-dPEG₁₂-acid (211 mg, 0.294 mmol) and N,N,N′,N′-Tetramethyl-O-(7-azabenzotriazol-1-yl)uronium hexafluorophos-phate (122 mg, 0.320 mmol) were dissolved in a chloroform/methanol/H₂O (6 mL, 65:35:8) in a 20 mL scintillation vial flushed with argon. N,N-Diisopropylethylamine (116 μL, 0.668 mmol) was added. Reaction was allowed to stir at 25° C. for 4 hours and concentrated. Material was then purified via silica gel chromatography with a dichloromethane/methanol gradient to yield (2R)-3-4((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate as an oil (252 mg, 65%).

### Preparation of DSPE-PEG550-VA

### (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate

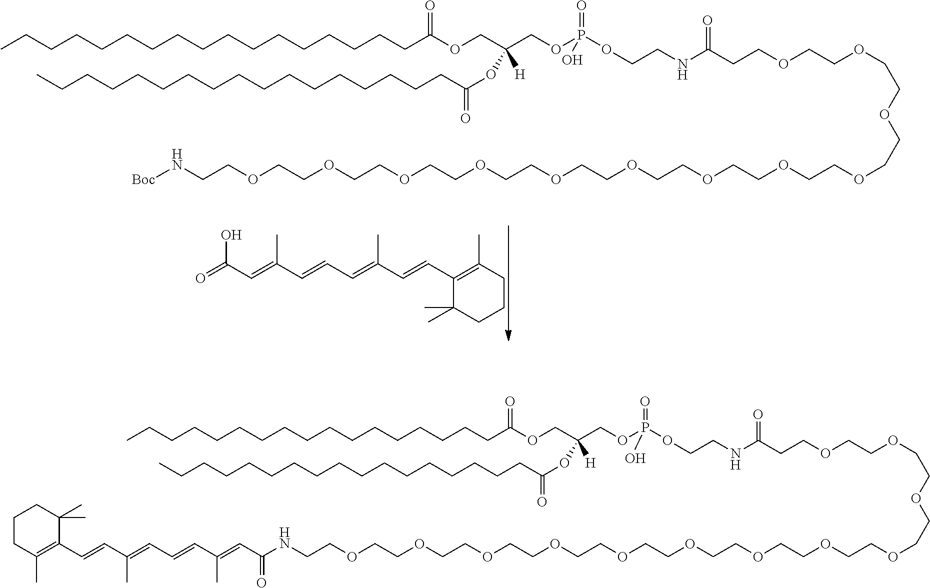 [SMILES: CCCCCCCCCCCCCCCCCC(=O)OC[C@H](COP(=O)(O)OCCNC(=O)CCOCCOCCOCCOCCOCCOCCOCCOCCOCCOCCOCCOCCNC(=O)OC(C)(C)C)OC(=O)CCCCCCCCCCCCCCCCC ; CC1=C(/C=C/C(C)=C/C=C/C(C)=C/C(=O)O)C(C)(C)CCC1 ; CCCCCCCCCCCCCCCCCC(=O)OC[C@H](COP(=O)(O)OCCNC(=O)CCOCCOCCOCCOCCOCCOCCOCCOCCOCCOCCOCCOCCNC(=O)/C=C(C)/C=C/C=C(C)/C=C/C1=C(C)CCCC1(C)C)OC(=O)CCCCCCCCCCCCCCCCC]

(2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate (252 mg, 0.174 mmol) was dissolved in diethyl ether (5 mL). Reaction was placed in a H₂O bath at room temperature. 2 M HCl/diethyl ether (2 mL, 4 mmol) was added and the mixture was allowed to stir for approximately 1 hour. Afterwards, solvent and excess HCl were removed in vacuo. Suspended material in 2 mL N,N-Dimethylformamide in a round bottom flask flushed with argon. Retinoic acid (57.5 mg, 0.191 mmol), N,N,N′,N′-Tetramethyl-O-(7-azabenzotriazol-1-yl)uronium hexafluorophosphate (79 mg, 0.209 mmol) and N,N-Diisopropylethylamine (106 μL, 0.609 mmol) were added. The material did not fully dissolve thus added more chloroform/methanol/H₂O (1 mL, 65:35:8 v:v:v mixture) to get reaction homogeneous. After 3.5 hours, the reaction mixture was concentrated. Material was then purified via silica gel chromatography with a dichloromethane/methanol gradient to yield (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate as a tan solid (210 mg, 74%). Verified product by NMR & LCMS. ¹H NMR (400 MHz), δ_{H}: 8.6 (s, 1H), 8.25 (d, 1H), 6.8-6.9 (dd, 1H), 6.3-6.4 (m, 1H), 6.12-6.25 (dd, 5H), 5.71 (s, 1H), 5.18 (m, 2H), 4.33 (dd, 2H), 4.13 (m, 2H), 3.95 (m, 2H), 3.74 (m, 8H), 3.63 (s, ˜48H), 3.0 (q, 2H), 2.5 (t, 3H), 2.35 (s, 3H), 2.25 (t, 8H), 1.97 (m, 7H), 1.7 (3, 3H), 1.5 (m, 2H), 1.36 (m, 12H), 1.23 (m, ˜56H), 1.01 (s, 6H), 0.86 (t, 12H). MS: m/z 1630.28 (M+H⁺).

### Example 15

### DSPE-PEG2000-Glu-VA

### Preparation of DSPE-PEG2000-Glu-VA

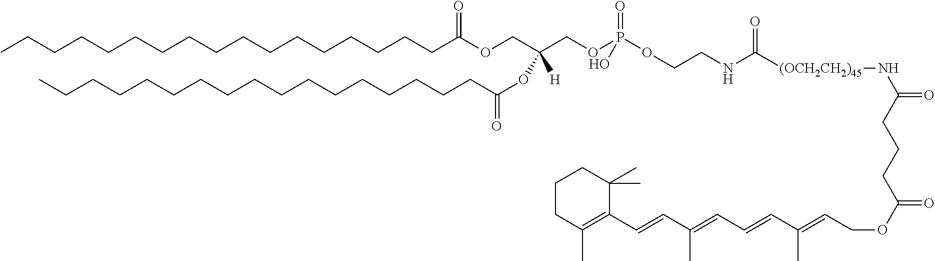

### Preparation of Intermediate 1

### 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

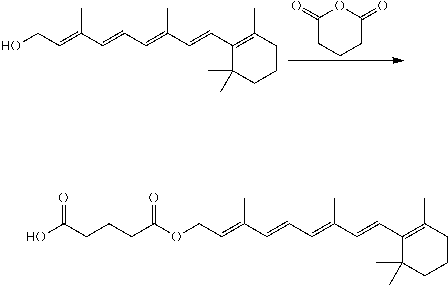 [SMILES: CC1=C(/C=C/C(C)=C/C=C/C(C)=C/CO)C(C)(C)CCC1 ; O=C1CCCC(=O)O1 ; CC1=C(/C=C/C(C)=C/C=C/C(C)=C/COC(=O)CCCC(=O)O)C(C)(C)CCC1]

Glutaric anhydride (115 mg, 1.01 mmol) and retinol (240 mg, 0.838 mmol) were dissolved in dichloromethane (3 mL) in an amber-colored vial. Triethylamine (257 μl, 1.84 mmol) was added and the vial was flushed with argon. Reaction was allowed to stir at room temperature overnight. The reaction mixture was concentrated and then purified via silica gel chromatography with a dichloromethane/methanol gradient to yield 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid as a yellowish oil (700 mg, 78%). Material characterized by NMR.

### Preparation of DSPE-PEG2000-Glu-VA

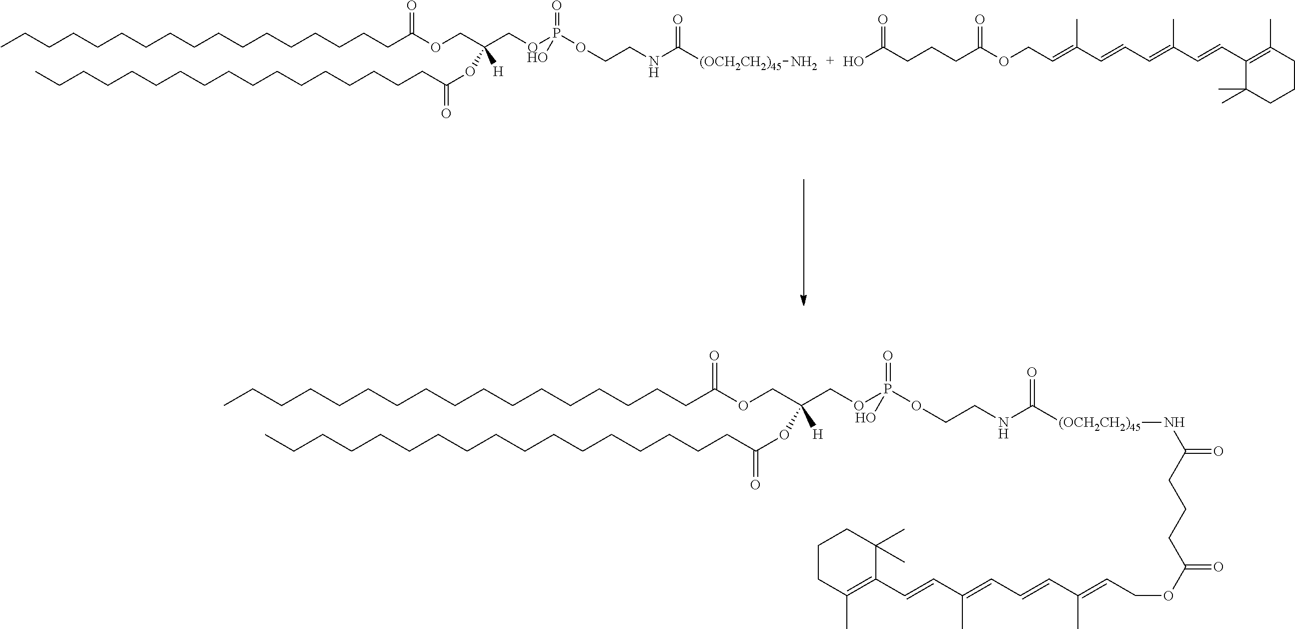

5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (43 mg, 0.108 mmol), DSPE-PEG2000-NH₂ (250 mg, 0.090 mmol) and N,N,N′,N′-tetramethyl-O-(7-azabenzotriazol-1-yl)uronium hexafluorophosphate (45 mg, 0.117 mmol) were dissolved in N,N-dimethylformamide (2 mL) in an amber-colored scintillation vial flushed with argon gas. N,N-diisopropylethylamine (47 μL, 0.270 mmol) was added and the reaction was allowed to stir overnight at room temperature, then purified via silica gel chromatography with a dichloromethane/methanol gradient to yield yellowish oil (59 mg, 20.7%). Verified product by NMR. ¹H NMR (400 MHz), δ_{H}: 706 (m, 1H), 6.59-6.66 (dd, 1H), 6.06-6.30 (m 5H), 5.56-5.60 (t, 1H), 5.17-5.23 (m, 2H), 4.35-4.42 (dd, 2H), 4.12-4.25 (m, 5H), 3.96-3.97 (m, 6H), 3.79-3.81 (t, 1H), 3.66 (m, ˜180H), 3.51-3.58 (m, 2H), 3.4-3.48 (m, 4H), 3.3-3.38 (m, 2H), 2.25-2.45 (m, 14H), 1.5-2.0 (m, 15H), 1.23-1.32 (m, ˜56H), 1.01 (s, 3H), 0.85-0.88 (t, 12H).

### Example 16

### DOPE-Gly₃-VA

### Preparation of (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate (DOPE-Gly₃-VA)

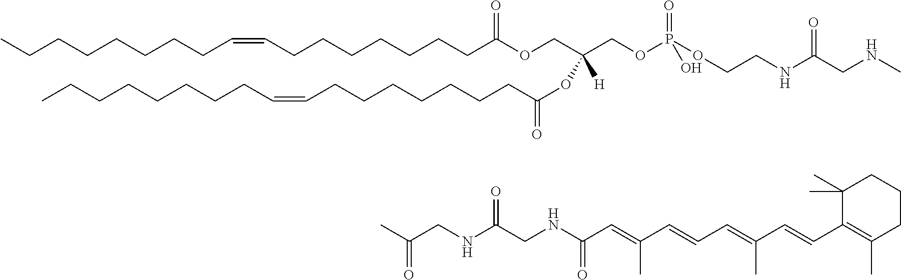

### Preparation of Intermediate 1

### (Z)-(2R)-3-(((2-(2-(2-(2-aminoacetamido)acetamido)acetamido)ethoxy)(hydroxy)phosphoryl)oxy)propane-1,2-diyl dioleate

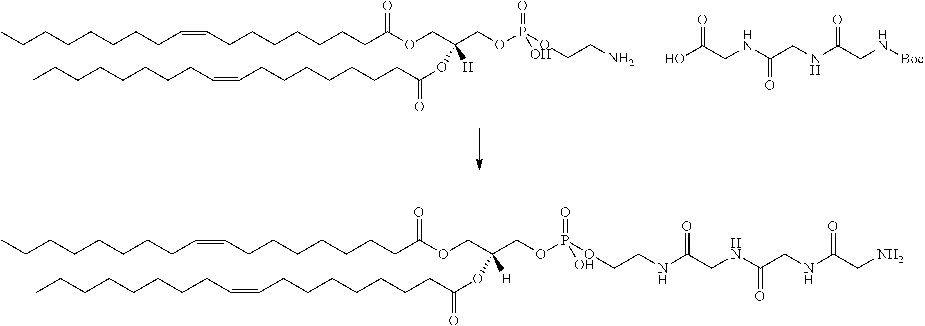

Boc-Gly-Gly-Gly-OH (382 mg, 1.34 mmol) and N,N,N′,N′-Tetramethyl-O-(7-azabenzotriazol-1-yl)uronium hexafluorophosphate (532 mg, 1.4 mmol) were dissolved in DMF (5 mL). N,N-Diisopropylethylamine (488 μL, 2.8 mmol) was added and the mixture was allowed to stir at room temperature for 10-15 minutes. Afterwards, a solution of 1,2-dioleoyl-sn-glycero-3-phosphoethanolamine (833 mg, 1.12 mmol) in chloroform (5 mL) was added and the reaction vessel was flushed with argon. After 16 hours at room temperature, the reaction mixture was concentrated and partitioned between dichloromethane (50 mL) and H₂O (50 mL), extracted with dichloromethane (3×50 mL), dried with sodium sulfate, filtered and concentrated. Material was purified via silica gel chromatography using a dichloromethane/methanol gradient to yield colorless oil residue. To this, 2 M HCl/Diethyl Ether (5 mL) was added and the reaction mixture was allowed to stir in a H₂O bath for approximately 2 hours. The reaction mixture was concentrated and the residue was taken up in dichloromethane (75 mL), washed with saturated sodium bicarbonate solution (75 mL), extracted product with dichloromethane (3×75 mL), dried with sodium sulfate, filtered and concentrated to yield (Z)-(2R)-3-(((2-(2-(2-(2-aminoacetamido)acetamido)-acetamido)ethoxy)(hydroxy)phosphoryl)oxy)propane-1,2-diyl dioleate as a semi-solid (765 mg, 90%). Verified by NMR.

### Preparation of DOPE-Gly₃-VA

### (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate

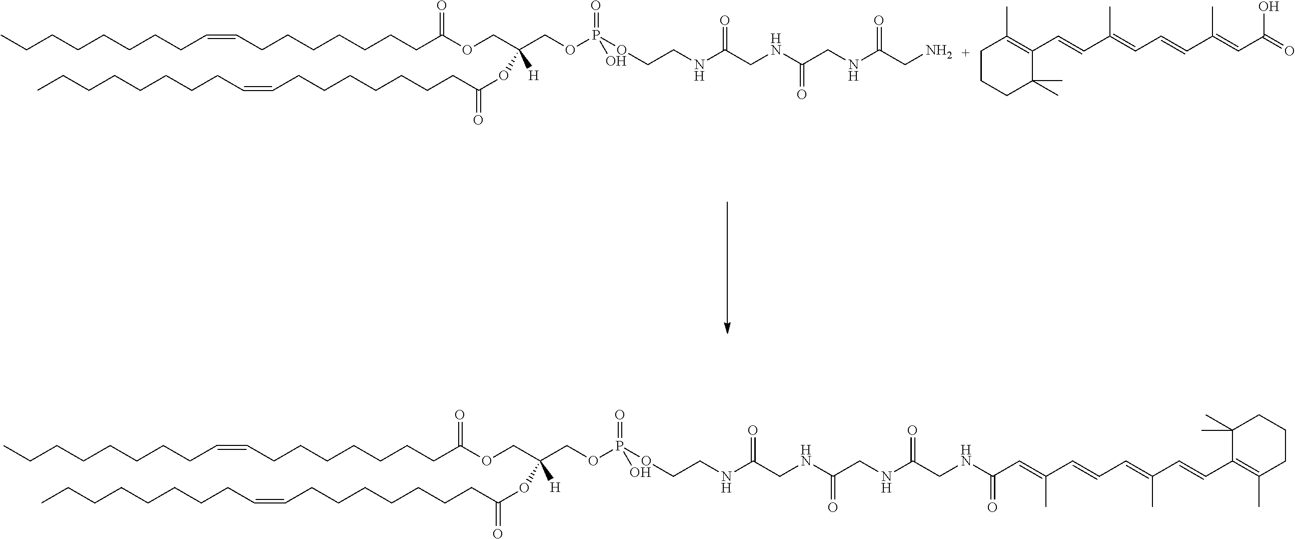

(Z)-(2R)-3-(((2-(2-(2-(2-aminoacetamido)acetamido)acetamido)ethoxy)-(hydroxy)phosphoryl)oxy)propane-1,2-diyl dioleate (765 mg, 0.836 mmol), retinoic acid (301 mg, 1.00 mmol), and N,N,N′,N′-Tetramethyl-O-(7-azabenzotriazol-1-yl)uronium hexafluorophosphate (413 mg, 1.09 mmol) were suspended in N,N-Dimethylformamide (5 mL). N,N-Diisopropylethylamine (437 μL, 2.51 mmol) was added and the reaction vessel was flushed with argon gas. Added chloroform (5 mL) to aid in the solvation of materials. Reaction was allowed to stir for ˜4 hours at room temperature in a round bottom flask covered with aluminum foil. Partitioned material between H₂O (100 mL) and dichloromethane (100 mL). Extracted with dichloromethane (3×100 mL), dried with sodium sulfate, filtered and concentrated. Material was then purified via silica gel chromatography using a dichloromethane/methanol gradient to yield (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)-propane-1,2-diyl dioleate as an orange oil (704 mg, 70%). Verified product by LCMS & NMR. ¹H NMR (400 MHz), δ_{H}: 6.90 (t, 1H), 6.21 (q, 2H), 6.08-6.12 (d, 2H), 5.83 (s, 1H), 5.31 (s, 4H), 5.30 (s, 2H), 4.37 (d, 1H), 4.15 (m, 1H), 3.91 (m, 8H), 3.59 (m, 2H), 3.29 (m, 2H), 3.01 (m, 2H), 2.28 (m, 6H), 1.95-1.98 (m, 12H), 1.44 (s, 3H), 1.5-1.6 (m, 2H), 1.44 (m, 6H), 1.24 (m, ˜48H), 1.00 (s, 6H), 0.86 (t, 3H). MS: m/z 1198.42 (M+H⁺).

### Example 17

### VA-PEG-VA

### Preparation of N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA)

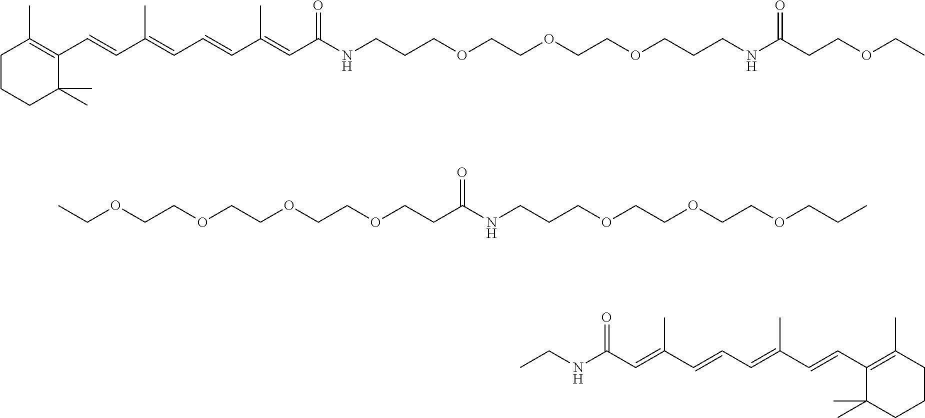

### Preparation of VA-PEG-VA

### N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide

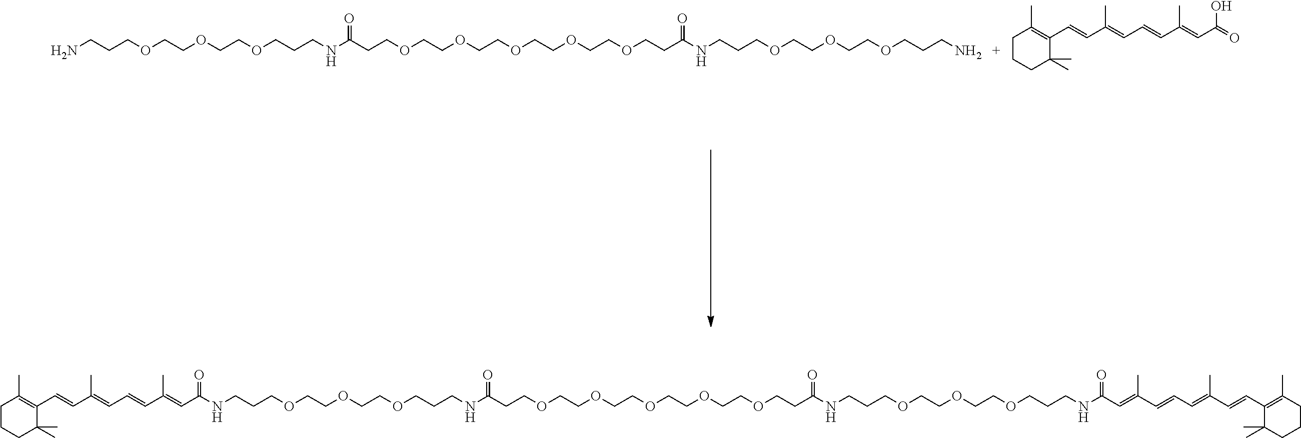

Retinoic acid (2913 mg, 9.70 mmol), N,N,N′,N′-Tetramethyl-O-(7-azabenzotriazol-1-yl)uronium hexafluorophosphate (3992 mg, 10.50 mmol) and diamido-dPEG₁₁-diamine (3000 mg, 4.04 mmol) were suspended in N,N-dimethylformamide (10 mL). N,N-Diisopropylethylamine (4222 μL, 24.24 mmol) was added and the vessel was flushed with argon. Reaction was allowed to stir at room temperature overnight in a round bottom flask covered with aluminum foil. Next day, partitioned material between ethyl acetate (125 mL) and water (125 mL). Extracted with ethyl acetate (3×125 mL), dried with sodium sulfate, filtered and concentrated. Material was then purified via silica gel chromatography with a dichloromethane/methanol gradient. Pooled fractions and concentrated to yield yellow oil (2900 mg, 54.9%). Verified product by LCMS & NMR. ¹H NMR (400 MHz), δ_{H}: 7.1 (s, 2H), 6.87 (t, 2H), 6.51 (t, 2H), 6.12-6.20 (dd, 8H), 5.66 (s, 2H), 3.6-3.8 (m, ˜44H), 3.4 (q, 4H), 3.3 (q, 4H), 2.46 (t, 4H), 2.32 (s, 6H), 1.9-2.05 (m, 10H), 1.7-1.85 (m, 15H), 1.6 (m, 4H), 1.3-1.5 (m, 6H), 1.01 (s, 12H). QTOF MS: m/z 1306 (M+H⁺).

### Example 18

### VA-PEG2000-VA

### Preparation of (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) (VA-PEG2000-VA)

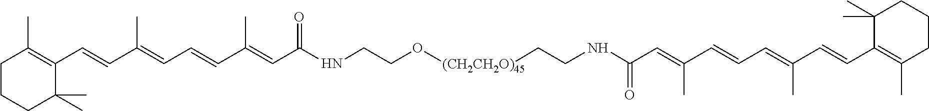

### Preparation of VA-PEG2000-VA

### (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide)

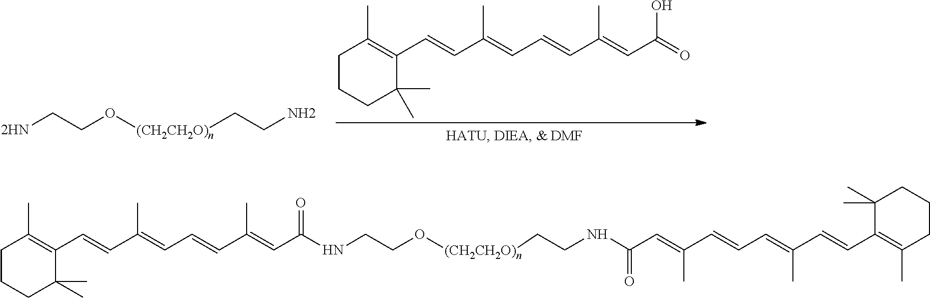

Retinoic acid (109 mg, 0.362 mmol), N,N,N′,N′-Tetramethyl-O-(7-azabenzotriazol-1-yl)uronium hexafluorophosphate (149 mg, 0.392 mmol) and amine-PEG_{2K}-amine (333 mg, 0.151 mmol) were suspended in N,N-Dimethylformamide (3 mL). N,N-Diisopropylethylamine (158 μL, 0.906 mmol) was added and the vessel was flushed with argon. Reaction was allowed to stir at room temperature overnight in a round bottom flask covered with aluminum foil. Next day, partitioned material between ethyl acetate (30 mL) and water (30 mL). Extracted with ethyl acetate (3×30 mL), dried with sodium sulfate, filtered and concentrated. Material was then purified via silica gel chromatography with a dichloromethane/methanol gradient. Pooled fractions and concentrated to yield (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) as a yellow oil (97 mg, 23%). Verified product by LCMS & NMR. ¹H NMR (400 MHz), δ_{H}: 6.85-6.92 (t, 2h), 6.20-6.32 (M, 6H), 6.08-6.12 (d, 4H), 5.72 (s, 2H), 3.55-3.70 (m, ˜180H), 3.4-3.5 (m, 4H), 2.79 (m, 4H), 2.78 (s, 6H), 2.33 (s, 6H), 2.05 (m, 4H), 1.97 (s, 6H), 1.80 (m, 2H), 1.79 (s, 6H), 1.69 (s, 6H), 1.60 (m, 4H), 1.45 (m, 4H), 1.01 (s, 12H). QTOF MS: m/z 2651 (M+H⁺).

### Example 19

### DSPE-PEG2000-VA

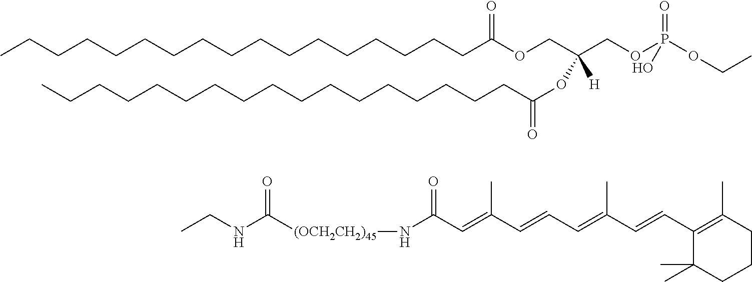

### Preparation of DSPE-PEG2000-VA

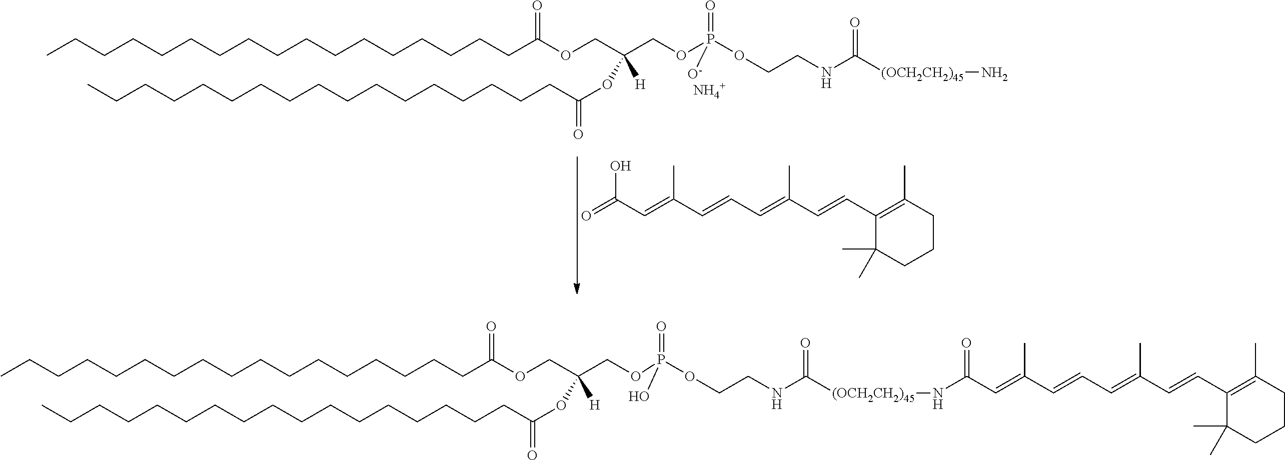

DSPE-PEG2000-NH₂ (250 mg, 0.090 mmol), retinoic acid (33 mg, 0.108 mmol) and N,N,N′,N′-Tetramethyl-O-(7-azabenzotriazol-1-yl)uronium hexafluorophosphate (45 mg, 0.117 mmol) were dissolved in N,N-Dimethylformamide. N,N-Diisopropylethylamine (47 μL, 0.270 mmol) was added to the mixture. The amber colored scintillation vial was flushed with argon and allowed to stir 3 days at room temperature. Material was then purified silica gel chromatography using a dichloromethane/methanol gradient. Pooled fractions and concentrated to yield DSPE-PEG2000-VA as a yellow oil (245 mg, 89%). Verified product by NMR. ¹H NMR (400 MHz), δ_{H}: 6.86 (dd, 1H), 6.25 (m, 1H), 6.09-6.21 (dd, 4H), 5.71 (s, 1H), 5.1-5.2 (m, 1H), 4.3-4.4 (d, 1H), 4.1-4.2 (m, 3H), 3.85-4.0 (m, 4H), 3.8 (t, 1H), 3.5-3.75 (m, ˜180H), 3.4-3.5 (m, 8H), 3.3 (m, 2H), 2.35 (s, 3H), 2.26 (m, 4H), 1.70 (s, 3H), 1.55-1.65 (m, 6H), 1.47 (m, 2H), 1.23 (s, ˜60H), 1.01 (s, 6H), 0.85 (t, 6H).

### Example 20

### diVA-PEG-diVA, Also Known as “DiVA”

### Preparation of N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA)

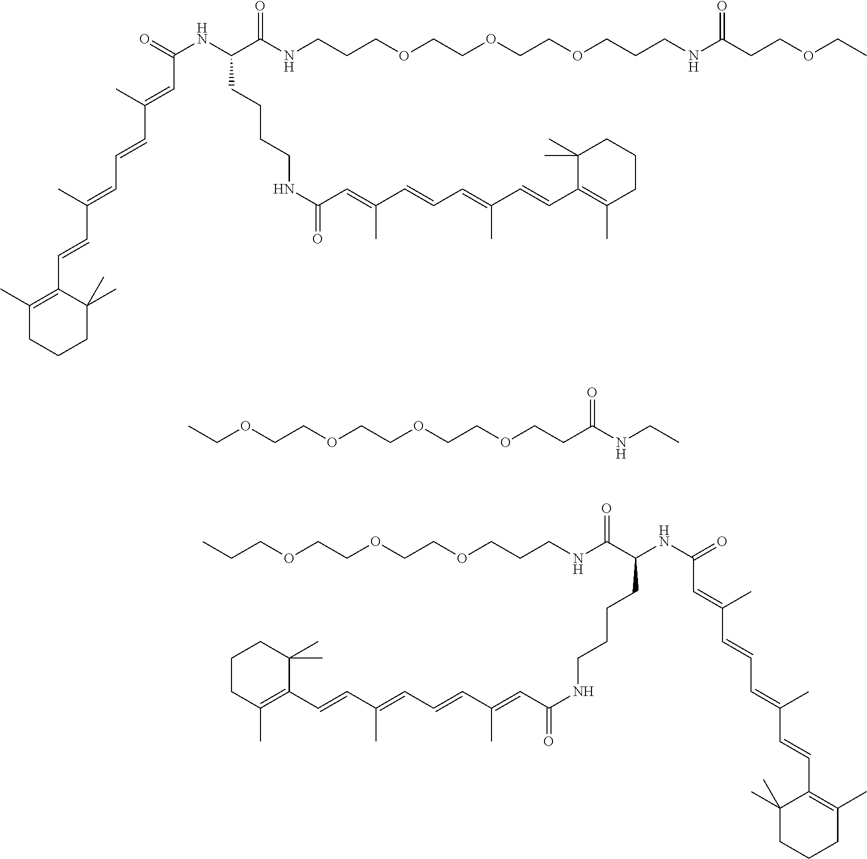

### Preparation of Intermediate 1

### tetrabenzyl ((5S,57S)-6,22,40,56-tetraoxo-11,14,17,25,28,31,34,37,45,48,51-undecaoxa-7,21,41,55-tetraazahenhexacontane-1,5,57,61-tetrayl)tetracarbamate, also known as Z-DiVA-PEG-DiVA-IN

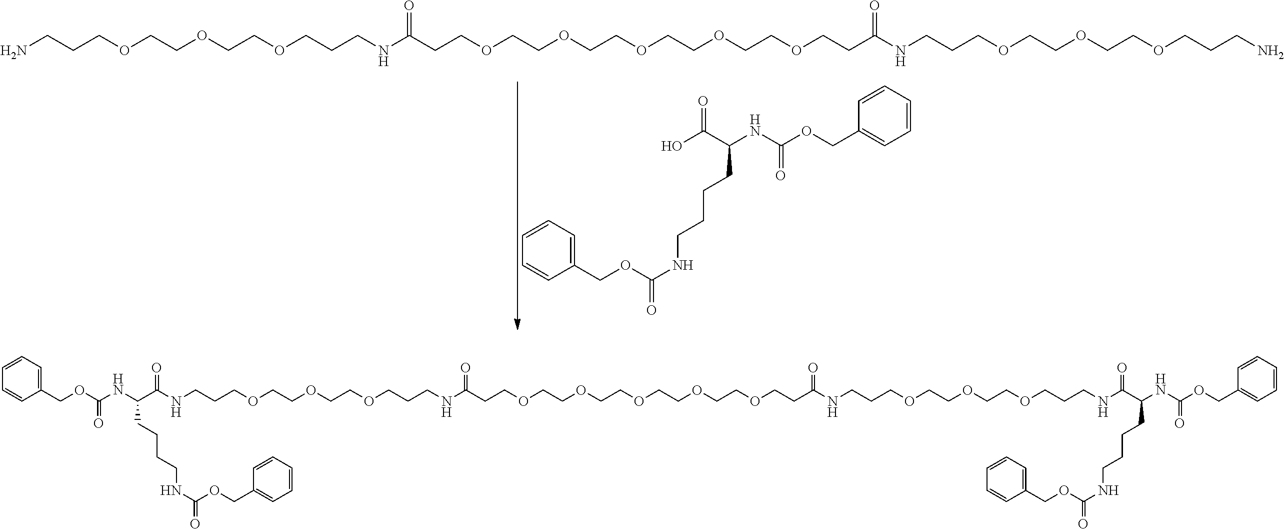 [SMILES: NCCCOCCOCCOCCCNC(=O)CCOCCOCCOCCOCCOCCC(=O)NCCCOCCOCCOCCCN ; O=C(NCCCC[C@H](NC(=O)OCc1ccccc1)C(=O)O)OCc1ccccc1 ; O=C(CCOCCOCCOCCOCCOCCC(=O)NCCCOCCOCCOCCCNC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)OCc1ccccc1]

A 1 L reaction flask cooled to 5-10° C. was purged with nitrogen and charged with dichloromethane (300 mL), d-PEG-11-diamine (Quanta lot EK1-A-1100-010, 50.0 g, 0.067 mol), Z-(L)-Lys(Z)—OH (61.5 g, 0.15 mol), and HOBt hydrate (22.5 g, 0.15 mol). 4-Methylmorpholine (4-MMP) (15.0 g, 0.15 mol) was added to the suspension and a light exothermic reaction was observed. A suspension of EDC hydrochloride (43.5 g, 0.23 mol) and 4-MMP (20.0 g, 0.20 mol) in dichloromethane (150 mL) was added over a period of 30 minutes, and moderate cooling was required in order to maintain a temperature of 20-23° C. The slightly turbid solution was stirred overnight at ambient temperature, and HPLC indicates completion of reaction. Deionized water (300 mL) was added and after having stirred for 10 minutes, a quick phase separation was observed. The aqueous phase was extracted with dichloromethane (150 mL)—with a somewhat slower phase separation. The combined organic extracts are washed with 6% sodium bicarbonate (300 mL) and dried with magnesium sulphate (24 g). Evaporation from a 40-45° C. water bath under reduced pressure gives 132 g of crude product. A solution of crude product (131 g) in 8% methanol in ethyl acetate in loaded onto a column of Silica Gel 60 (40-630, packed with 8% methanol in ethyl acetate. The column was eluted with 8% methanol in ethyl acetate (7.5 L). The fractions containing sufficiently pure product (5.00-7.25 L) was evaporated from a 45° C. water bath under reduced pressure and 83.6 g of purified product. A solution of purified product (83.6 g) in dichloromethane (200 mL) was loaded onto a column of Dowex 650 C (H⁺) (200 g), which has been washed with dichloromethane (250 mL). The column was eluted with dichloromethane (200 mL). The combined product containing fractions (300-400 mL) were dried with magnesium sulphate (14 g) and evaporated from a 45° C. water bath under reduced pressure to yield tetrabenzyl ((5S,57S)-6,22,40,56-tetraoxo-11,14,17,25,28,31,34,37,45,48,51-undecaoxa-7,21,41,55-tetraazahenhexacontane-1,5,57,61-tetrayl)tetracarbamate, also known as Z-DiVA-PEG-DiVA-IN (77.9 g, HPLC purity 94.1%).

### Preparation of Intermediate 2

### N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN

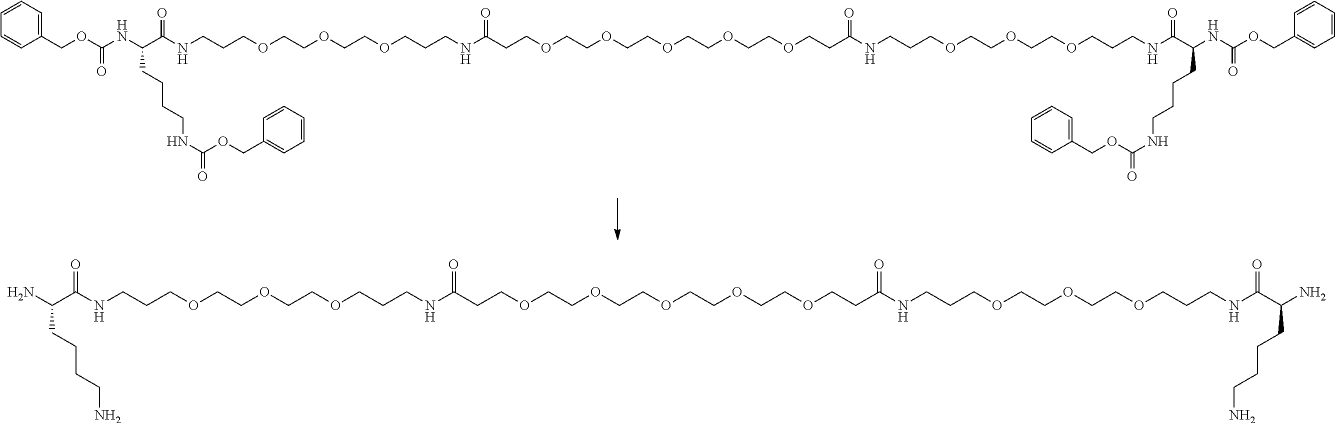 [SMILES: O=C(CCOCCOCCOCCOCCOCCC(=O)NCCCOCCOCCOCCCNC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)OCc1ccccc1 ; NCCCC[C@H](N)C(=O)NCCCOCCOCCOCCCNC(=O)CCOCCOCCOCCOCCOCCC(=O)NCCCOCCOCCOCCCNC(=O)[C@@H](N)CCCCN]

A 1 L reaction flask was purged with nitrogen and charged with methanol (600 mL) and Z-DiVA-PEG-DiVA-IN (92.9, 60.5 mmol). The mixture was stirred under nitrogen until a solution was obtained. The catalyst, 10% Pd/C/50% water (Aldrich, 10 g) was added. The mixture was evacuated, and then the pressure was equalized by nitrogen. The mixture was evacuated, and then the pressure was equalized by hydrogen. Ensuring a steady, low flow of hydrogen over the reaction mixture, the stirrer was started. Hydrogenation was continued in a flow of hydrogen for one hour. The system was then closed, and hydrogenation was continued at ˜0.1 bar for one hour. The mixture was evacuated and then re-pressurized to ˜0.1 bar with hydrogen. After another hour of hydrogenation, the mixture was evacuated and then re-pressurized to 0.1 bar with hydrogen. Stirring under hydrogen was continued for 15 hours after which time no starting material could be detected by HPLC. The mixture was evacuated, and then the pressure was equalized by nitrogen. The mixture was evacuated, and then the pressure was equalized by nitrogen. The reaction mixture was then filtered on a pad of celite 545. The filter cake was washed with methanol (100 mL). The combined filtrate was concentrated, finally at 45° C. and at a pressure of less than 50 mbar. Toluene (100 mL) was added and the resulting mixture was again concentrated finally at 45° C. and at a pressure of less than 40 mbar to yield N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN (63.4 g), as an oil that solidifies upon standing.

### Preparation of DiVA-PEG-DiVA

### N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-tri-methylcyclohex-1-en-1-yl)-4,7,10-trioxa-14,21-diazatriaconta-23,25,27,29-tetraen-1-yl)-4,7,10,13,16-pentaoxanonadecane-1,19-diamide

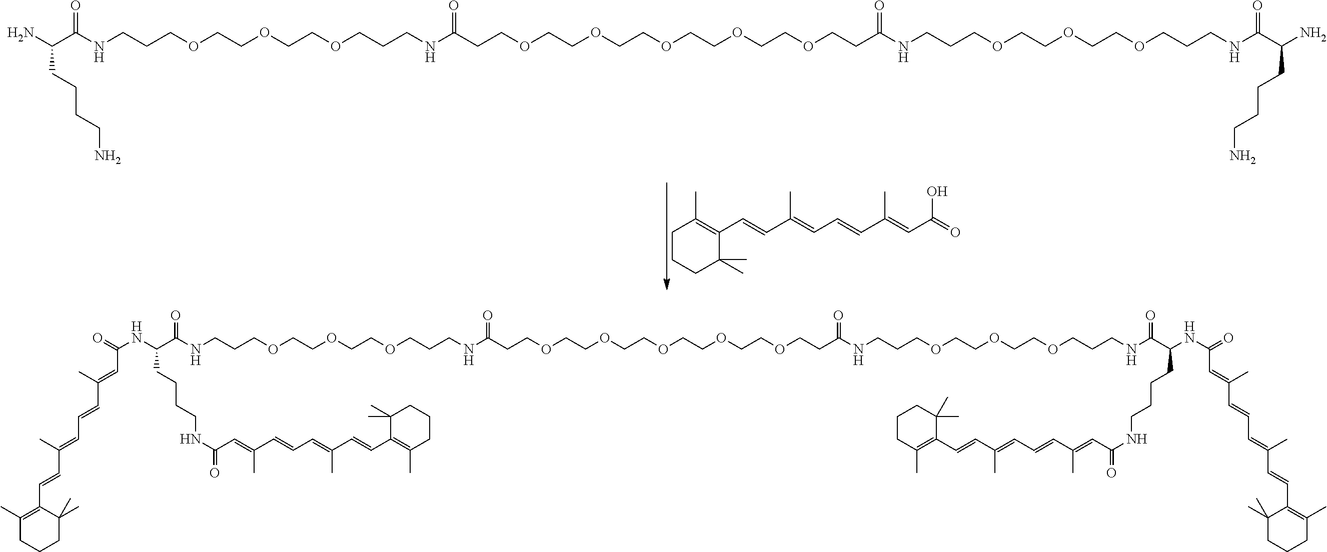

A 2 L reactor was filled with argon and charged with dichloromethane (500 mL), DiVA-PEG-DiVA-IN (52.3 g, 52.3 mmol), retinoic acid (70.6 g, 235 mmol) and 4-N,N-dimethylaminopyridine (2.6 g, 21.3 mmol). The mixture was stirred under argon until dissolved (˜20 minutes). Keeping the temperature of the reaction at 10-20° C., 1-ethyl-3-(3-dimethylaminopropyl) carbodiimide) (EDCI) (70.6 g, 369 mmol) was added portion wise over a period of 10-15 minutes (the reaction was slightly exothermic for the first 30-60 minutes). The reactor was covered with aluminium foil and the mixture was stirred at 18-21° C. for 15-20 hours. Butylated hydroxytoluene (BHT) (25 mg) was added and the reaction mixture was then poured onto aqueous 6% sodium hydrogen carbonate (500 mL) while keeping an argon atmosphere over the mixture. The organic phase was separated. The aqueous phase was washed with dichloromethane (50 mL). The combined organic phase was dried with of magnesium sulphate (150 g) under inert atmosphere and protected from light. The drying agent was filtered off (pressure filter preferred) and the filter cake was washed with dichloromethane (500 mL). The filtrate was concentrated by evaporation at reduced pressure using a water bath of 35-40° C. The oily residue was added toluene (150 mL) and evaporated again to yield a semi-solid residue of 210 g. This residue was dissolved in dichloromethane (250 mL) and applied onto a column prepared from silica gel 60 (1.6 kg) and 0.5% methanol in dichloromethane) (4 L). The column was eluted with dichloromethane (7.2 L), 2), 3% methanol in dichloromethane (13 L), 5% methanol in dichloromethane (13 L), 10% methanol in dichloromethane (18 L). One 10 L fraction was taken, and then 2.5 L fractions were taken. The fractions, protected from light were sampled, flushed with argon and sealed. The fractions taken were analyzed by TLC (10% methanol in dichloromethane, UV). Fractions holding DiVA-PEG-DiVA were further analyzed by HPLC. 5 Fractions <85% pure (gave 32 g of evaporation residue) were re-purified in the same manner, using only 25% of the original amounts of silica gel and solvents. The fractions >85% pure by HPLC were combined and evaporated at reduced pressure, using a water bath of 35-40° C. The evaporation residue (120 g) was re-dissolved in dichloromethane (1.5 L) and slowly passed (approximately 1 hour) through a column prepared from ion exchanger Dowex 650C, H⁺ form (107 g). The column was then washed with dichloromethane (1 L). The combined eluate (3277.4 g) was mixed well and a sample (25 mL, 33.33 g) was evaporated, finally at room temperature and a pressure of <0.1 mBar to afford 0.83 g of a foam. From this figure the total amount of solid material was thus calculated to a yield of 80.8 g (72.5%). The remaining 3.24 kg of solution was concentrated to 423 g. 266 g of this solution was concentrated further to yield a syrup and then re-dissolved in abs. ethanol (200 mL). Evaporation at reduced pressure, using a water bath of 35-40° C., was continued to yield a final ethanol solution of 94.8 g holding 50.8 g (53.6% w/w) of N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as DiVA-PEG-DiVA, also known as “DiVA”. Characterized by NMR & QTOF. ¹H NMR (400 MHz), δ_{H}: 7.07 (t, 2H), 7.01 (t, 2H), 6.87-6.91 (m, 4.0H), 6.20-6.24 (m, 10H), 6.10-6.13 (m, 8H), 5.79 (s, 2H), 5.71 (s, 2H), 4.4 (q, 2H), 3.70 (t, 6H), 3.55-3.65 (m, ˜34H), 3.59 (t, 6H), 3.4 (m, 2H), 3.25-3.33 (m, 10H), 3.16 (m, 2H), 2.44 (t, 4H), 2.33 (s, 12H), 1.97-2.01 (m, 12H), 1.96 (s, 6H), 1.7-1.9 (m, 12H), 1.69 (s, 12H), 1.5-1.65 (m, 12H), 1.35-1.5 (m, 24H), 1.01 (s, 24H). QTOF MS ESI+: m/z 2128 (M+H⁺).

### Example 21

### DOPE-VA

### Preparation of (Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (DOPE-VA)

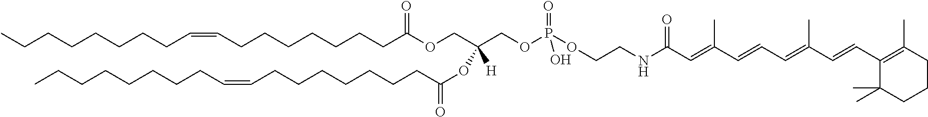 [SMILES: CCCCCCCC/C=C\CCCCCCCC(=O)OC[C@H](COP(=O)(O)OCCNC(=O)/C=C(C)/C=C/C=C(C)/C=C/C1=C(C)CCCC1(C)C)OC(=O)CCCCCCC/C=C\CCCCCCCC]

### Preparation of DOPE-VA

### (Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate

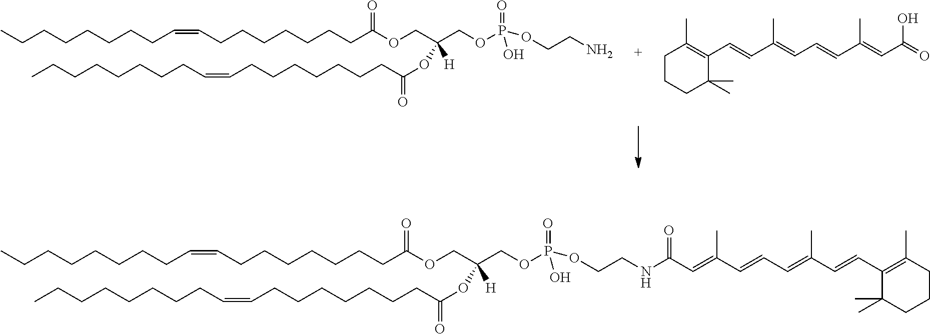 [SMILES: CCCCCCCC/C=C\CCCCCCCC(=O)OC[C@H](COP(=O)(O)OCCN)OC(=O)CCCCCCC/C=C\CCCCCCCC ; CC1=C(/C=C/C(C)=C/C=C/C(C)=C/C(=O)O)C(C)(C)CCC1 ; CCCCCCCC/C=C\CCCCCCCC(=O)OC[C@H](COP(=O)(O)OCCNC(=O)/C=C(C)/C=C/C=C(C)/C=C/C1=C(C)CCCC1(C)C)OC(=O)CCCCCCC/C=C\CCCCCCCC]

To a solution of retinoic acid (250 mg, 0.83 mmol) in diethyl ether stirring (20 mL) at −78° C., a solution of (diethylamino)sulfur trifluoride (130 μl, 0.90 mmol) in cold ether (20 mL) was added through a syringe. The reaction mixture was taken out of the cold bath and the stirring was continued at room temperature for an additional 2 hr. At the end, the solvent was removed by rotary evaporation. The residue was redissolved chloroform (50 mL) in the presence of solid Na₂CO₃(50 mg). To this solution was added 1,2-dioleoyl-sn-glycero-3-phosphoethanolamine (600 mg, 0.81 mmol) and the reaction mixture was stirred at room temperature for an additional 24 hrs. The solvent was removed by rotary evaporation. The residue was purified by silica gel chromatography with a dichloromethane/methanol gradient to yield Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (240 mg, 28%). ¹H NMR (400 MHz, CDCl₃) δ0.87 (t, 6H, CH₃), 1.01 (s, 6H, CH₃) 1.20-1.40 (m, 40H, CH₂), 1.40-1.60 (m, 8H, CH₂), 1.70 (s, 3H, CH₃—C═C), 1.80-2.10 (m, 8H), 2.32 (m, 4H, CH₂C(═O)), 3.50 (m, 2H), 3.92-4.18 (m, 5H), 4.35 (m, 2H), 5.20 (m, 1H, NHC(═O)), 5.31 (m, 4H, CH═CH), 5.80-6.90 (m, 6H, CH═CH).

### Example 22

### DC-VA

### Preparation of (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (DC-VA)

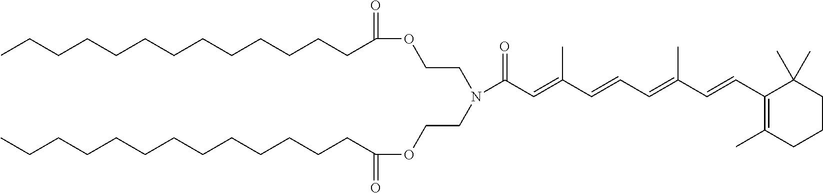 [SMILES: CCCCCCCCCCCCCC(=O)OCCN(CCOC(=O)CCCCCCCCCCCCC)C(=O)/C=C(C)/C=C/C=C(C)/C=C/C1=C(C)CCCC1(C)C]

### Preparation of DC-VA

### (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate

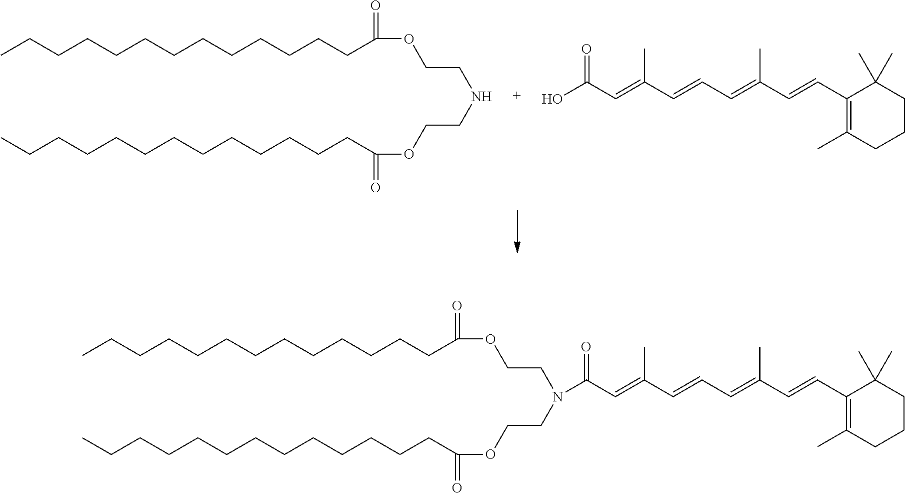 [SMILES: CCCCCCCCCCCCCC(=O)OCCNCCOC(=O)CCCCCCCCCCCCC ; CC1=C(/C=C/C(C)=C/C=C/C(C)=C/C(=O)O)C(C)(C)CCC1 ; CCCCCCCCCCCCCC(=O)OCCN(CCOC(=O)CCCCCCCCCCCCC)C(=O)/C=C(C)/C=C/C=C(C)/C=C/C1=C(C)CCCC1(C)C]

To a solution of retinoic acid (600 mg, 2.0 mmol) in diethyl ether (25 mL) stirring at −78° C., a solution of (diethylamino)sulfur trifluoride (0.3 ml, 2.1 mmol) in 5 mL of cold ether was added through a syringe. The reaction mixture was taken out of the cold bath and the stirring was continued at room temperature for an additional 1 hr. After the solvent was removed by rotary evaporation, the residue was re-dissolved in dichloromethane (20 mL) in the presence of 2 solid Na₂CO₃ (25 mg). To this solution was added the azanediylbis(ethane-2,1-diyl)ditetradecanoate (1.05 g, 2.0 mmol), and the reaction mixture was stirred at room temperature for an additional 24 hrs. The reaction mixture was diluted with dichloromethane (50 mL) and was dried over MgSO₄. After the solvent was removed by rotary evaporation, the residue was purified by silica gel chromatography with a dichloromethane/methanol gradient to yield (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (800 mg, 50%). ¹H NMR (400 MHz, CDCl₃) δ 0.87 (t, 6H, CH₃), 1.02 (s, 6H, CH₃) 1.20-1.40 (m, 40H, CH₂), 1.40-1.60 (m, 8H, CH₂), 1.70 (s, 3H, CH₃—C═C), 1.97 (s, 3H, CH₃—C═C), 2.05 (m, 2H, CH₂), 2.15 (s, 3H, CH₃—C═C), 2.32 (m, 4H, CH₂C(═O)), 3.67 (m, 4H, NCH₂CH₂O), 4.15-4.30 (m, 4H, NCH₂CH₂O), 5.80-6.90 (m, 6H, CH═CH).

### Example 23

### DC-6-VA

### Preparation of ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl)ditetradecanoate (DC-6-VA)

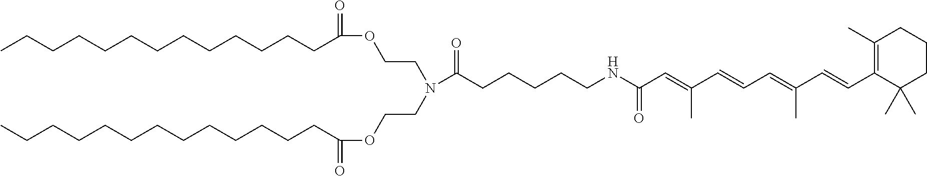 [SMILES: CCCCCCCCCCCCCC(=O)OCCN(CCOC(=O)CCCCCCCCCCCCC)C(=O)CCCCCNC(=O)/C=C(C)/C=C/C=C(C)/C=C/C1=C(C)CCCC1(C)C]

### Preparation of Intermediate 1

### ((6-aminohexanoyl)azanediyl)bis(ethane-2,1-diyl)ditetradecanoate TFA salt

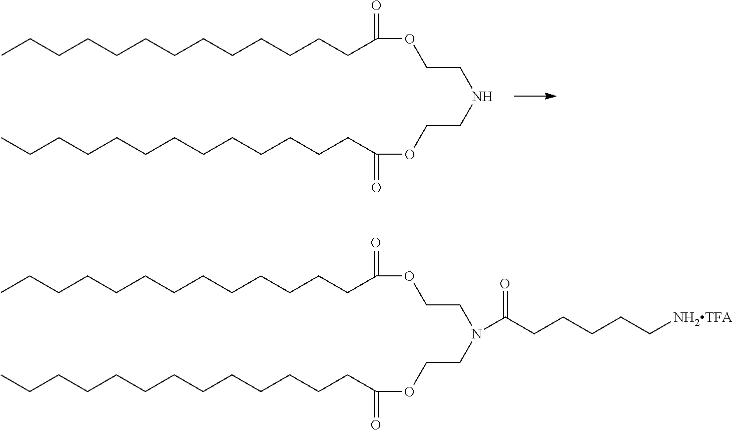

A mixture of azanediylbis(ethane-2,1-diyl)ditetradecanoate (2.5 g, 4.8 mmol), Boc-amino caproic acid (1.3 g, 5.6 mmol), N,N′-dicyclohexylcarbodiimide (1.3 g, 6.3 mmol) and N,N-diisopropylethylamine (2.6 mL, 0.015 mmol) were dissolved in pyridine (40 mL). The solution was stirred at 60° C. for overnight. The mixture was diluted with dichloromethane (50 mL) and washed with saline (3×50 mL). After being concentrated by rotary evaporation, the residue was treated with trifluoroacetic acid/dichloromethane (100 mL, 1:1). The mixture was concentrated and was re-dissolved in dichloromethane (50 mL) and washed with saline (3×50 mL). The organic layer was isolated and concentrated to yield ((6-aminohexanoyl)azanediyl)bis(ethane-2,1-diyl)ditetradecanoate TFA salt (1.5 g, 33%).

### Preparation of DC-6-VA

### ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl)

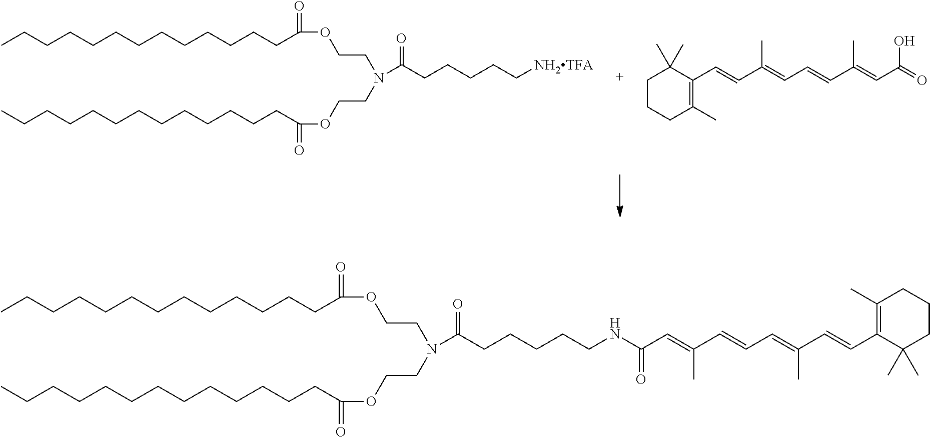

To a solution of retinoic acid (800 mg, 2.67 mmol) in diethyl ether (40 mL) stirring at −78° C., a solution of (diethylamino)sulfur trifluoride (0.4 mL, 22.80 mmol) in cold ether (7 mL) was added through a syringe. The reaction mixture was taken out of the cold bath and the stirring was continued at room temperature for an additional 1 hr. After the solvent was removed by rotary evaporation, the residue was re-dissolved in dichloromethane (25 mL) in the presence of solid Na₂CO₃ (40 mg). To this solution was added the ((6-aminohexanoyl)azanediyl)bis(ethane-2,1-diyl)ditetradecanoate TFA salt (1.5 g, 1.6 mmol) and the reaction mixture was stirred at room temperature for an additional 24 hrs. The reaction mixture was diluted with dichloromethane (50 mL) and dried over MgSO₄. After the solvent was removed by rotary evaporation, the residue was purified by column chromatography using 5% methanol/dichloromethane as eluent to yield ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl) (360 mg, 24%). ¹H NMR (400 MHz, CDCl₃) δ 0.87 (t, 6H, CH₃), 1.02 (s, 6H, CH₃) 1.20-1.40 (m, 42H, CH₂), 1.40-1.60 (m, 12H, CH₂), 1.70 (s, 3H, CH₃—C═C), 1.97 (s, 3H, CH₃—C═C), 2.05 (m, 2H, CH₂), 2.15 (s, 3H, CH₃—C═C), 2.32 (m, 6H, CH₂C(═O)), 3.20 (m, 2H, CH₂NHC(═O)), 3.56 (m, 4H, NCH₂CH₂O), 4.15-4.30 (m, 4H, NCH₂CH₂O), 5.10 (m, 1H), 5.80-6.90 (m, 6H, CH═CH).

### Example 24

### In Vitro Evaluation of VA-siRNA-Liposome Formulations for Knockdown Efficiency in LX-2 Cell Line and Rat Primary Hepatic Stellate Cells (pHSCs)

LX2 cells (Dr. S. L. Friedman, Mount Sinai School of Medicine, NY) were grown in DMEM (Invitrogen) supplemented with 10% fetal bovine serum (Invitrogen) at 37° C. in the incubator with 5% CO₂. Cells were trypsinized using TryPLExpress solution (Invitrogen) for 3 min at 37° C. in the incubator. The cell concentration was determined by cell counting in hemocytometer and 3000 cells/well were seeded into the 96-well plates. The cells were grown for 24 h prior to transfection.

Rat primary hepatic stellate cells (pHSCs) were isolated from Sprague-Dawley rats according to the previously published method (Nat. Biotechnol. 2008, 26(4):431-42). pHSCs were grown in DMEM supplemented with 10% fetal bovine serum. Cells were grown up to two passages after isolation before using them for in vitro screening. Cells were seeded at the cell density of 1000 cells/well in 96-well plates and grown for 48 h before using them for transfection.

Transfection with VA-siRNA-Liposome formulations: The transfection method is the same for LX-2 and pHSC cells. The VA-siRNA-Liposome or VA-siRNA-Lipoplex formulations were mixed with growth medium at desired concentrations. 100 μl of the mixture was added to the cells in 96-well plate and cells were incubated for 30 min at 37° C. in the incubator with 5% CO₂. After 30 min, medium was replaced with fresh growth medium after. After 48 h of transfection, cells were processed using Cell-to-Ct lysis reagents (Applied Biosystems) according to the manufacturer's instructions.

Quantitatve (q) RT-PCR for measuring HSP47 mRNA expression: HSP47 and GAPDH TaqMan® assays and One-Step RT-PCR master mix were purchased from Applied Biosystems. Each PCR reaction contained the following composition: One-step RT-PCR mix 5 μl, TaqMan® RT enzyme mix 0.25 μl, TaqMan® gene expression assay probe (HSP47) 0.25 TaqMan® gene expression assay probe (GAPDH) 0.5 RNase-free water 3.25 μl, Cell lysate 0.75 μl, Total volume of 10 μl. GAPDH was used as endogenous control for the relative quantification of HSP47 mRNA levels. Quantitative RT-PCR was performed in ViiA™ 7 realtime PCR system (Applied Biosciences) using an in-built Relative Quantification method. All values were normalized to the average HSP47 expression of the mock transfected cells and expressed as percentage of HSP47 expression compared to mock.

The siRNA referred to in the formulation protocols are double stranded siRNA sequence with 21-mer targeting HSP47/gp46 wherein HSP47 (mouse) and gp46 (rat) are homologs—the same gene in different species:

Rat HSP47-C Double Stranded siRNA Used for In Vitro Assay (Rat pHSCs)

(SEQ. ID NO. 1)
Sense (5′->3′) GGACAGGCCUCUACAACUATT
(SEQ. ID NO. 2)
Antisense (3′->5′) TTCCUGUCCGGAGAUGUUGAU.

Cationic Lipid Stock Preparation: Stock solutions of cationic lipids were prepared by combining the cationic lipid with DOPE, cholesterol, and diVA-PEG-DiVA in ethanol at concentrations of 6.0, 5.1 and 2.7 and 2.4 mg/mL respectively. If needed, solutions were warmed up to about 50° C. to facilitate the dissolution of the cationic lipids into solution.

Empty Liposome Preparation: A cationic lipid stock solution was injected into a rapidly stirring aqueous mixture of 9% sucrose at 40±1° C. through injection needle(s) at 1.5 mL/min per injection port. The cationic lipid stock solution to the aqueous solution ratio (v/v) is fixed at 35:65. Upon mixing, empty vesicles formed spontaneously. The resulting vesicles were then allowed to equilibrate at 40° C. for 10 minutes before the ethanol content was reduced to ˜12%.

Lipoplex Preparation: The empty vesicle prepared according to the above method was diluted to the final volume of 1 mM concentration of cationic lipid by 9% sucrose. To the stirring solution, 100 μL of 5% glucose in RNase-free water was added for every mL of the diluted empty vesicle (“EV”) and mixed thoroughly. 150 μL of 10 mg/mL siRNA solution in RNase-free water was then added at once and mixed thoroughly. The mixture was then diluted with 5% glucose solution with 1.750 mL for every mL of the EV used. The mixture was stirred at about 200 rpm at room temperature for 10 minutes. Using a semi-permeable membrane with 100000 MWCO in a cross-flow ultrafiltration system using appropriately chosen peristaltic pump (e.g. Midgee Hoop, UFP-100-H24LA), the mixture was concentrated to about ⅓ of the original volume (or desired volume) and then diafiltered against 5 times of the sample volume using an aqueous solution containing 3% sucrose and 2.9% glucose. The product was then filtered through a combined filter of 0.8/0.2 micron pore size under aseptic conditions before use.

Formation of non-diVA siRNA containing liposomes: Cationic lipid, DOPE, cholesterol, and PEG conjugated lipids (e.g., Peg-Lipid) were solubilized in absolute ethanol (200 proof) at a molar ratio of 50:10:38:2. The siRNA was solubilized in 50 mM citrate buffer, and the temperature was adjusted to 35-40° C. The ethanol/lipid mixture was then added to the siRNA-containing buffer while stirring to spontaneously form siRNA loaded liposomes. Lipids were combined with siRNA to reach a final total lipid to siRNA ratio of 15:1 (wt:wt) The range can be 5:1 to 15:1, preferably 7:1 to 15:1. The siRNA loaded liposomes were diafiltered against 10× volumes of PBS (pH 7.2) to remove ethanol and exchange the buffer. Final product was filtered through 0.22 μm, sterilizing grade, PES filter for bioburden reduction. This process yielded liposomes with a mean particle diameter of 50-100 nm, PDI<0.2, >85% entrapment efficiency.

Formation of siRNA containing liposomes co-solubilized with diVA: siRNA-diVA-Liposome formulations were prepared using the method described above. diVA-PEG-diVA was co-solubilized in absolute ethanol with the other lipids (cationic lipid, DOPE, cholesterol, and PEG-conjugated lipids at a ratio of 50:10:38:2) prior to addition to the siRNA containing buffer. Molar content of diVA-PEG-diVA ranged from 0.1 to 5 molar ratio. This process yielded liposomes with a mean particle diameter of 50-100 nm, PDI<0.2, >85% entrapment efficiency.

Formation of siRNA containing liposomes with cationic lipids: siRNA-diVA-Liposome formulations and siRNA-Liposome formulations were prepared using the method described above. Cationic lipid can be, for example, DODC, HEDC, HEDODC, DC-6-14, or any combination of these cationic lipids.

Formation of siRNA containing liposomes decorated with diVA: siRNA-Liposome formulations were prepared using the method described above and diluted to a siRNA concentration of 0.5 mg/mL in PBS. Cationic lipid can be DODC, HEDC, HEDODC, DC-6-14, or any combination of these cationic lipids. diVA-PEG-diVA was dissolved in absolute ethanol (200 proof) to a final concentration ranging from 10 to 50 mg/mL. An appropriate amount of ethanol solution was added to the siRNA-Liposome solution to yield a final molar percentage between 2 to 10 mol %. Solution was plunged up and down repeatedly with a pipette to mix. diVA-PEG-diVA concentration and ethanol addition volume were adjusted to keep the addition volume >1.0 μL and the final ethanol concentration <3% (vol/vol). Decorated liposomes were then gently shaken at ambient temperature for 1 hr on an orbital shaker prior to in vitro or in vivo evaluation.

### Results

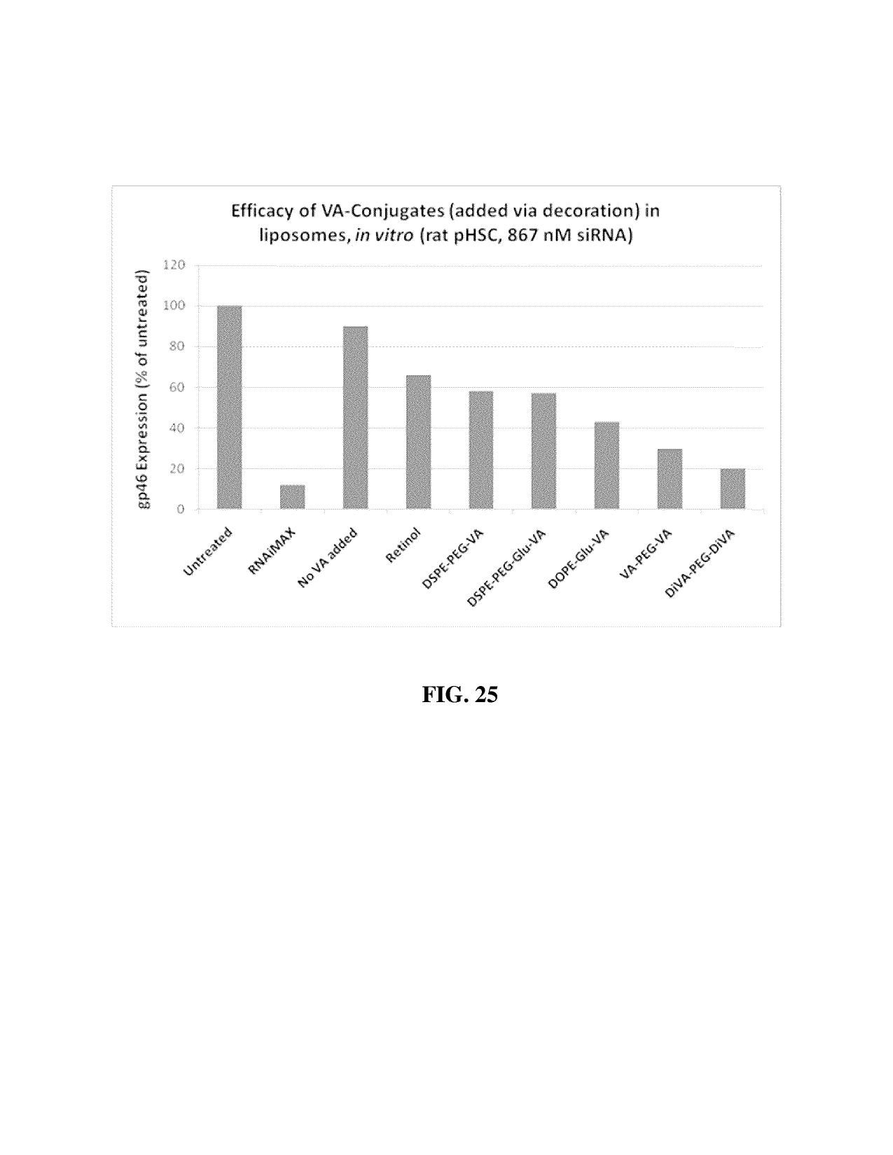

FIG. 25 shows that addition of the VA-conjugate to liposomes via decoration improved the knockdown efficacy of siRNA, enhancing siRNA activity. Peg-Lipid. The dose for all samples was 867 nM siRNA HSP47-C. The results showed that in every instance where a VA-conjugate was added to liposomes, siRNA activity was enhanced compared to liposomes without a retinoid and compared to liposomes decorated with free (non-conjugated) retinol. RNAiMAX was a commercial transfection reagent.

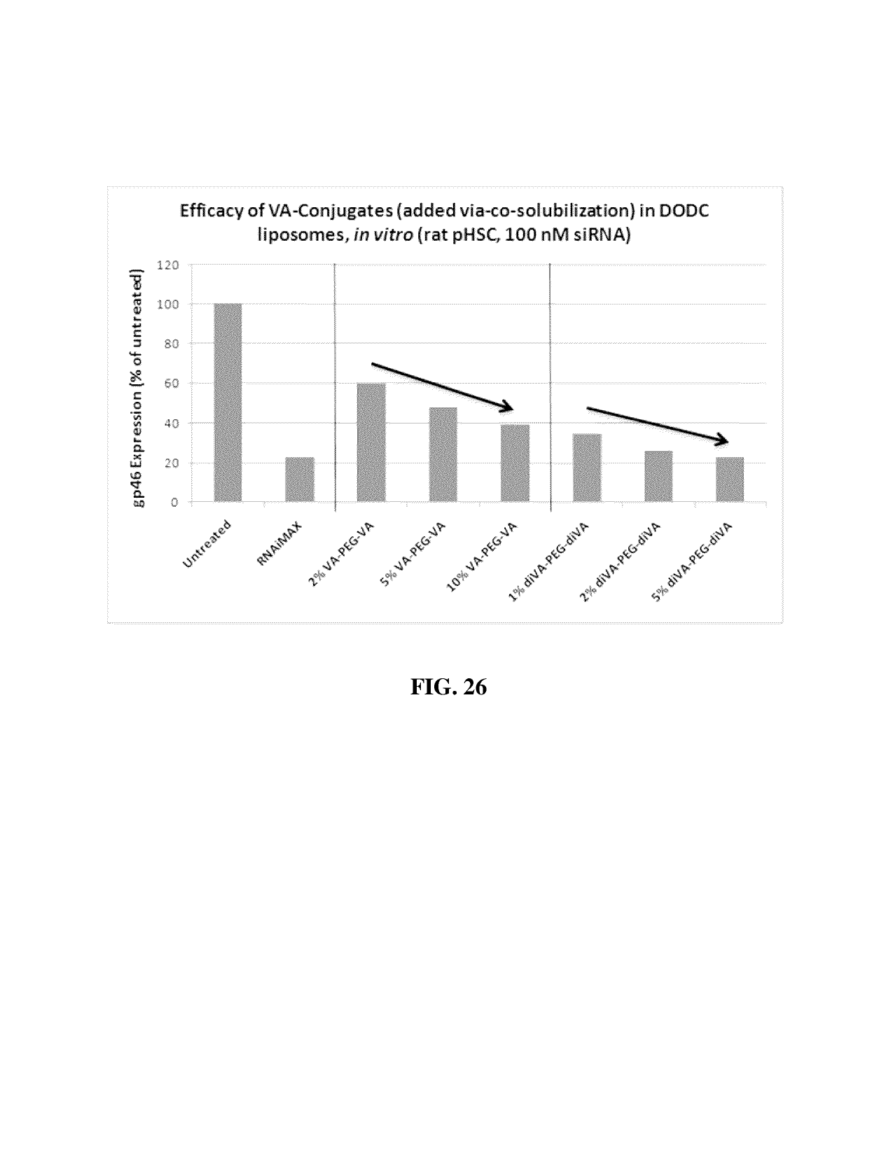

FIG. 26 shows that addition of VA-conjugates to liposomes via co-solubilization improves knockdown efficacy of siRNA. These were DODC containing liposomes with VA-conjugates added via co-solubilization. The formulation is 50:10:38:2:X, where X=1 to 10 (DODC:DOPE:cholesterol:PEG-Lipid:VA-conjugate, mole ratio). The concentration in every instance was 100 nM siRNA HSP47-C. The results show that addition of VA-conjugates to liposomes via cosolubilization enhances siRNA activity.

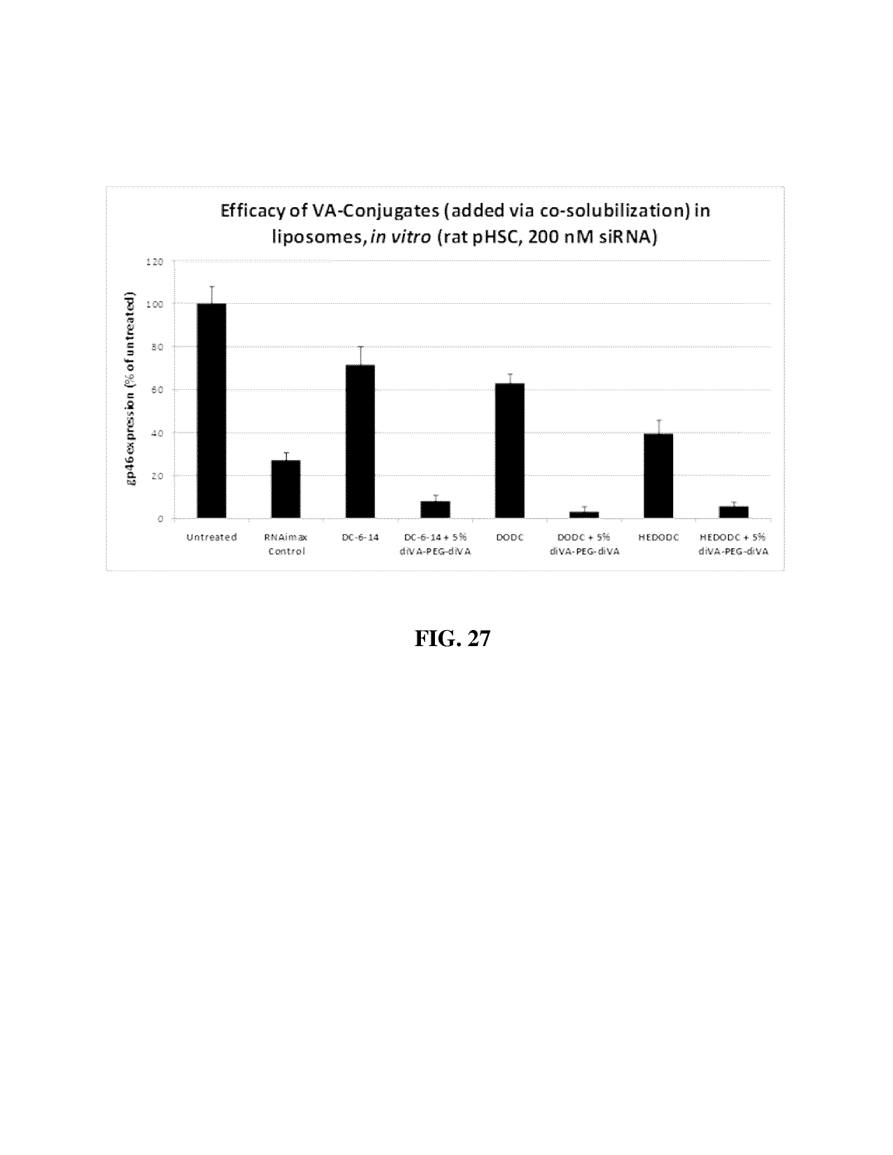

FIG. 27 shows that addition of VA-conjugate to liposomes via co-solubilization dramatically improves the knockdown efficacy of siRNA. Results include three different liposomes, DC-6-14, DODC, HEDODC with VA-conjugates added via co-solubilization. The formulation is the same for all, 50:10:38:2, cationic lipid:DOPE:cholesterol:Peg-Lipid, with only the cationic lipid varying. The concentration of siRNA is 200 nM siRNA HSP47-C is the same for all. The results show in that VA-conjugate addition to liposomes having different cationic lipids significantly enhanced siRNA activity, when prepared by co-solubilization.

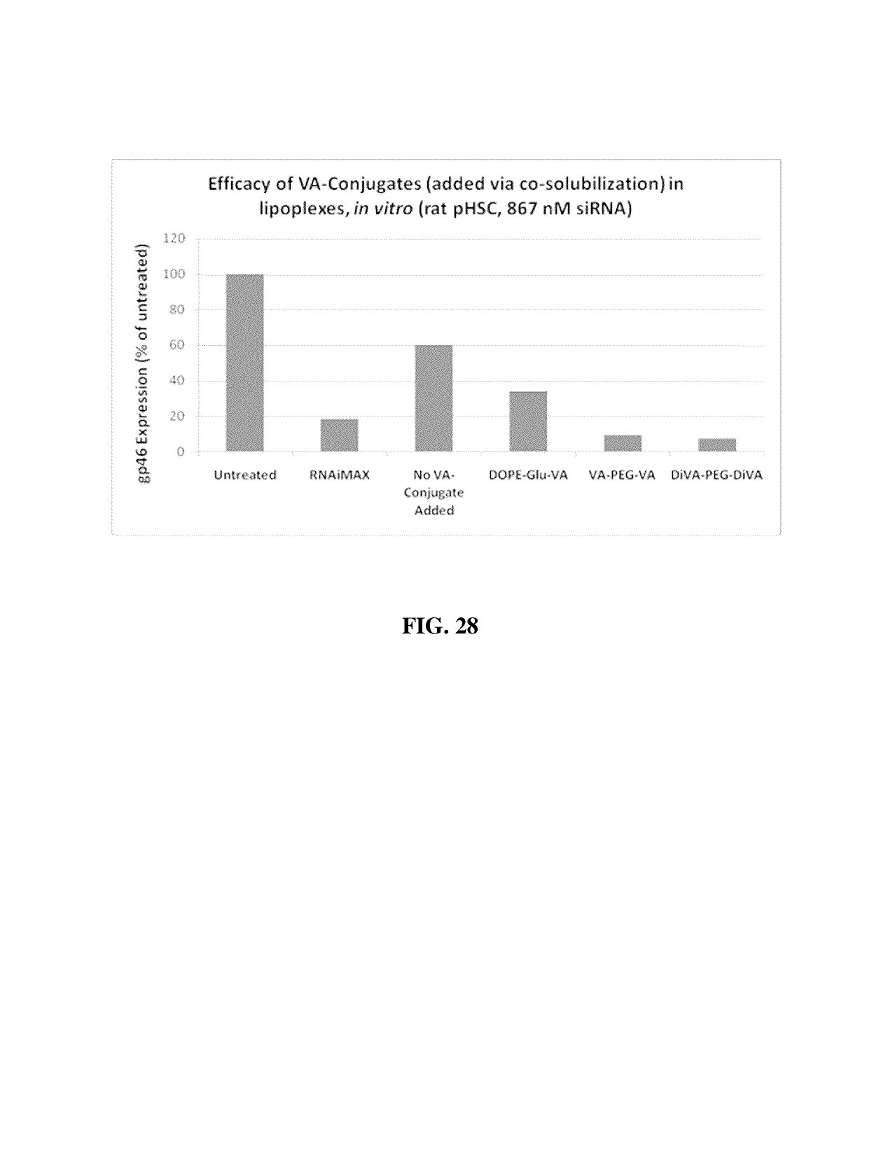

FIG. 28 shows that addition of VA-conjugates to lipoplexes having DC-6-14 cationic lipid via co-solubilization, and siRNA coating the exterior of the liposome enhances siRNA activity. The formulation is a 40% lipoplex formulation, 40:30:30, DC-6-14:DOPE:cholesterol. The concentration for all samples is 867 nM siRNA HSP47-C. The results show that VA-conjugate addition to lipoplexes via co-solubilization enhance siRNA activity.

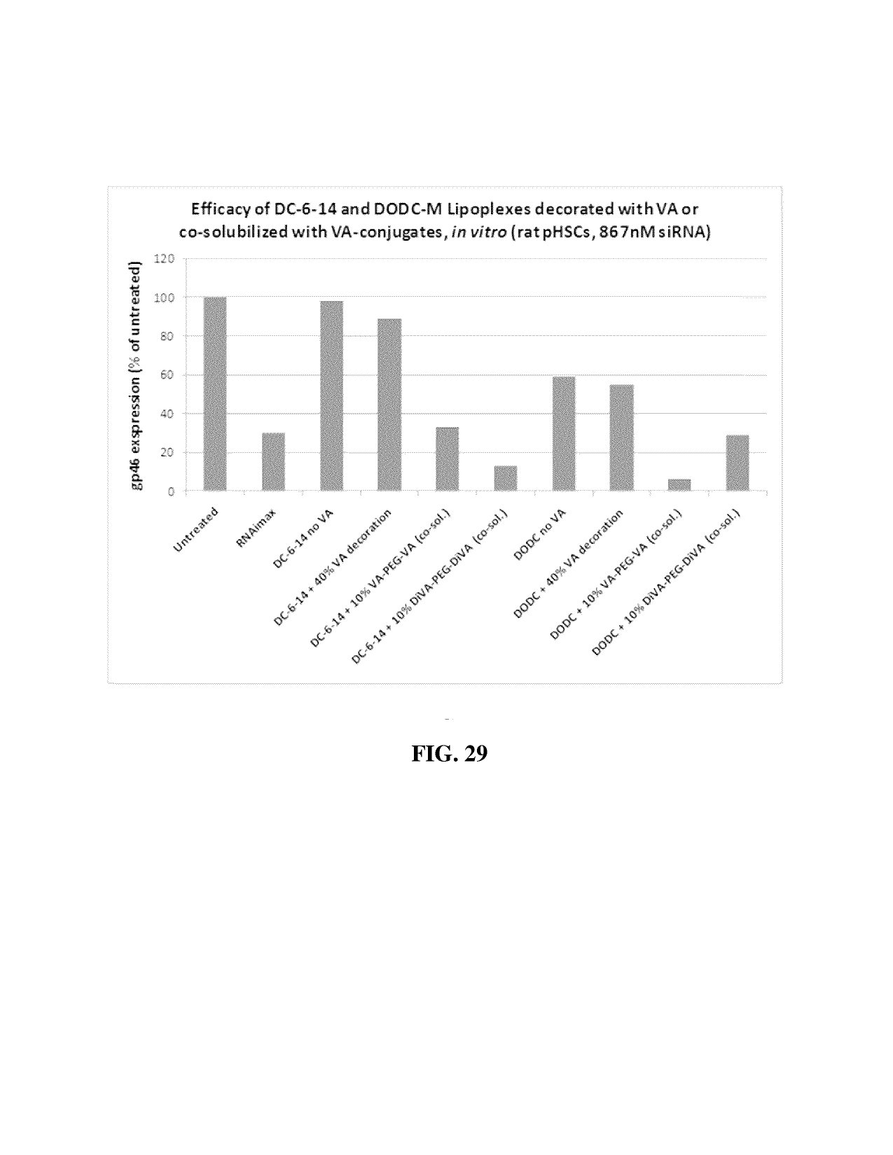

FIG. 29 shows that addition of VA-conjugate to lipoplexes formed via co-solubilization compared to lipoplexes with VA-conjugate added via decoration. These results are from DC-6-14 and DODC lipoplexes. The formulation consists of 40:30:30, DC-6-14:DOPE:cholesterol. The concentration in each sample is 867 nM siRNA HSP47-C. VA-conjugate addition via co-solubilization significantly improves knockdown efficacy in vitro, relative to VA-conjugates added by decoration.

### Example 25

### In Vivo Experiments

Female C57Bl/6 retired breeder mice (Charles River) with a weight range of 24-30 grams were used. Animals were randomly distributed by weight into 10 groups of 10 animals each. All animal procedures were approved by Bio-Quant's IACUC and/or Attending Veterinarian as necessary and all animal welfare concerns were addressed and documented. Mice were anesthetized with Isoflurane and exsanguinated via the inferior vena cava.

Mouse HSP47-C Double Stranded siRNA Used in Formulations for In Vivo Assay (Mouse CCl4 Model)

(SEQ. ID NO. 3)
Sense (5′->3′) GGACAGGCCUGUACAACUATT
(SEQ. ID NO. 4)
Antisense (3′->5′) TTCCUGUCCGGACAUGUUGAU

Upregulation of heat shock protein 47 (HSP47) was induced via intraperitoneal injection of CCl₄ (CCl₄ in olive oil, 1:7 (vol/vol), 1 μL per gram body weight) given every other day for 7 days (day 0, 2, 4, 6). On day 3 mice were treated for 4 consecutive days (day 3, 4, 5, 6) with liposome or lipoplex formulations of the invention or PBS by IV injection into the tail vein. One group of ten mice (naïve) received neither CCl₄ treatment nor IV injection and served as the control group for normal HSP47 gene expression.

Experimental Timeline

| | Day | | | | | | | |
|---|---|---|---|---|---|---|---|---|
| | 0 | 1 | 2 | 3 | 4 | 5 | 6 | 7 |
| CCl₄ IP Injection | X | X | X | X | X | X | X | |
| Test Article IV | | | | X | X | X | X | |
| Injection | | | | | | | | |
| Sample Collection | | | | | | | | X |
| (n = 10) | | | | | | | | |

On day 7 and approximately 24 hours after final IV injection, all remaining mice were sacrificed and the livers were perfused with PBS prior to collecting liver samples for PCR analysis. An approximate 150 mg sample from each mouse liver was collected and placed in 1.5 mL RNAlater stabilization reagent (Qiagen) and stored at 2-8° C. until analysis. Liver samples were not collected from areas of clear and marked liver damage and/or necrosis.

Total RNA from mouse livers was extracted using RNeasy® columns (Qiagen) according to the manufacturer's protocol. 20 ng of total RNA was used for quantitative RT-PCR for measuring HSP47 expression. HSP47 and GAPDH TaqMan® assays and One-Step RT-PCR master mix were purchased from Applied Biosystems. Each PCR reaction contained the following composition: One-step RT-PCR mix 5 μl, TaqMan® RT enzyme mix 0.25 μl, TaqMan® gene expression assay probe (HSP47) 0.25 μl, TaqMan® gene expression assay probe (GAPDH) 0.5 RNase-free water 3.25 RNA 0.75 Total volume of 10 μl. GAPDH was used as endogenous control for the relative quantification of HSP47 mRNA levels. Quantitative RT-PCR was performed in ViiA™ 7 realtime PCR system (Applied Biosciences) using an in-built Relative Quantification method. All values were normalized to the average HSP47 expression of the naïve animal group and expressed as percentage of HSP47 expression compared to naïve group.

### Example 26

### Synthesis of satDiVA

### Preparation of N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide (satDIVA)

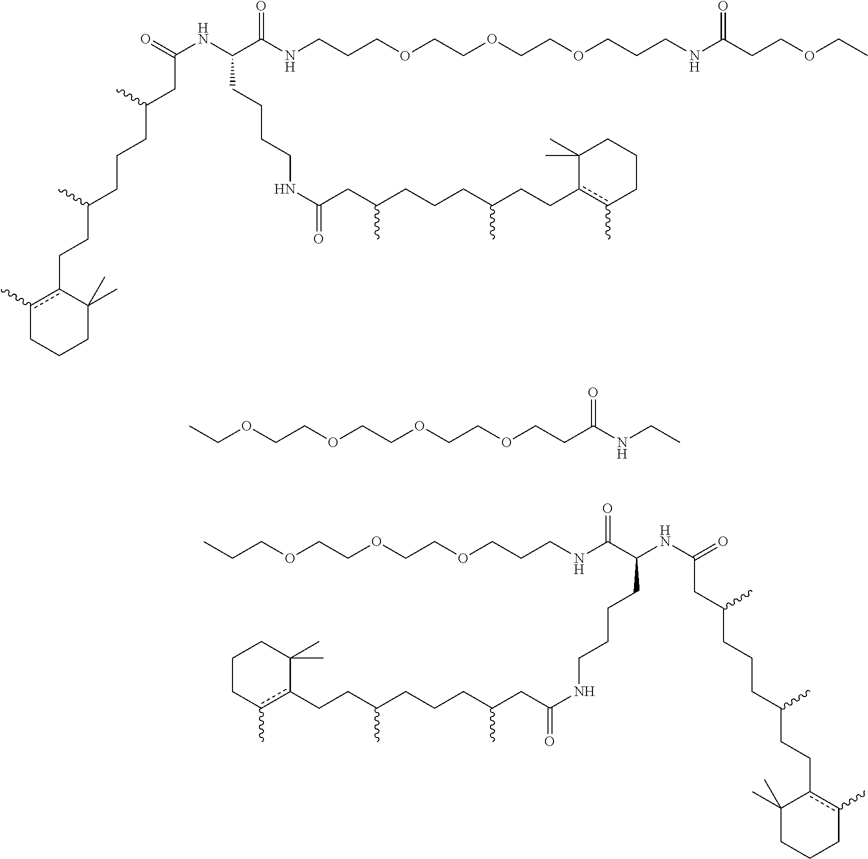

### Preparation of Intermediate 1

### 3,7-dimethyl-9-(2,6,6-trimethylcyclohex-1-en-1-yl)nonanoic acid

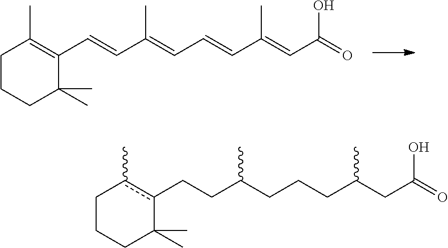

All-trans retinoic acid (2000 mg, 6.66 mmol) was dissolved in hexanes/IPA (3:1, 40 mL) with the aid of sonication. Material was placed in a Parr-shaker bottle and flushed with inert gas. 10% Pd/C (200 mg) was added and the vessel was once again flushed with inert gas. Material was placed on the Parr-Shaker overnight with >70 psi Hydrogen gas. The reaction mixture was then filtered through a pad of celite and concentrated to yield 3,7-dimethyl-9-(2,6,6-trimethylcyclohex-1-en-1-yl)nonanoic acid (2 g).

### Preparation of satDIVA

### N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide

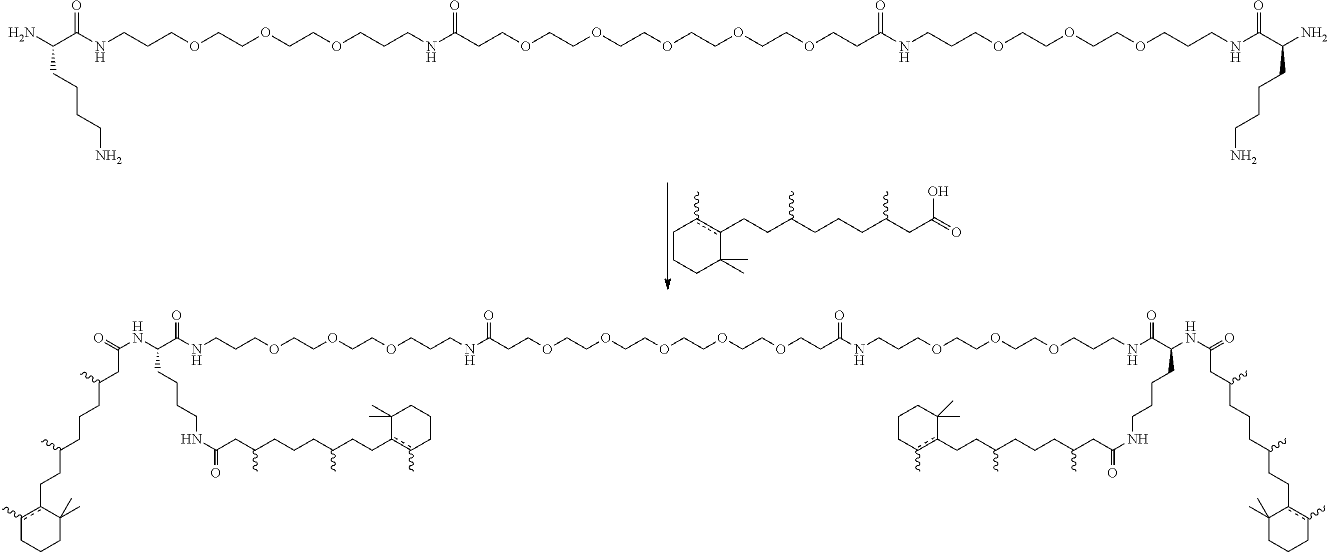

N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide, also known as satDIVA, was prepared in similar fashion as diva-PEG-diVA from previously described N1,N19-bis((S)-16,20-diamino-15-oxo-4,7,10-trioxa-14-azaicosyl)-4,7,10,13,16-pentaoxanonadecane-1,19-diamide with the substitution of 3,7-dimethyl-9-(2,6,6-trimethylcyclohex-1-en-1-yl)nonanoic acid for all-trans retinoic acid. QTOF MS ESI+: m/z 2161, 2163, 2165 & 2167 (M+H+)

### Example 27

### Synthesis of simDiVA

### Preparation of N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide (simDiVA)

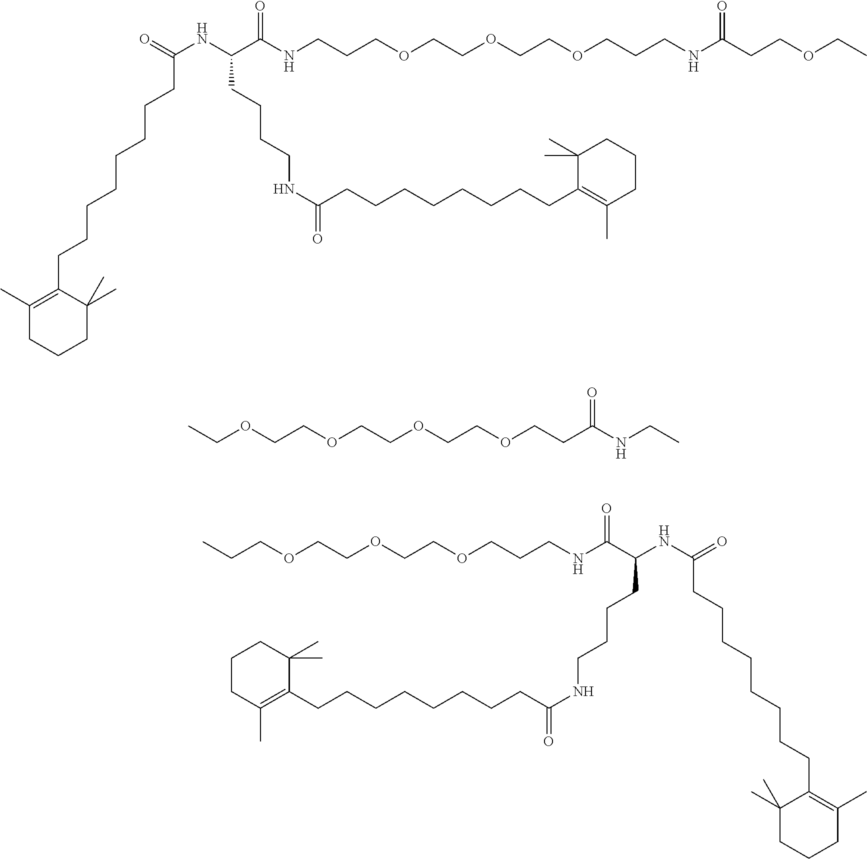

### Preparation of Intermediate 1

### 2,6,6-trimethylcyclohex-1-en-1-yl trifluoromethanesulfonate

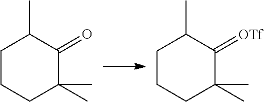

To a solution of 2,2,6-trimethylcyclohexanone in dry THF at −78° C. under nitrogen was added dropwise a 2 M lithium diisopropylamide solution. The mixture was stirred at −78° C. for 3 h. A solution of N-phenyl-bis(trifluoromethanesulfonimide) in THF was then added dropwise (at −78° C.). The reaction flask was packed in dry-ice and stirred overnight. The stirring was continued at room temperature for 3 h under which time all material had dissolved. The reaction mixture was concentrated and the residue was added slowly to hexane (350 mL) under vigorous stirring. The solid material was removed by filtration and washed with hexane (2×50 mL). The filtrate was concentrated and more hexane (150 mL) was added. The solid material was removed by filtration and the filtrate was concentrated. The precipitation was repeated one more time after which the residue was purified by flash chromatography (silica, hexane) to give 2,6,6-trimethylcyclohex-1-en-1-yl trifluoromethanesulfonate as a colorless oil (23.2 g, 60% yield).

### Preparation of Intermediate 2

### ethyl 9-(bromozincio)nonanoate

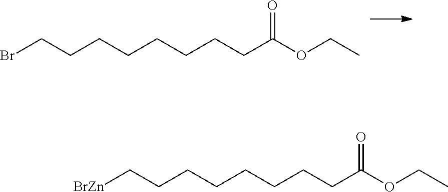

In a dry reaction tube under nitrogen were charged zinc dust (3.70 g, 56.6 mmol), iodine (479 mg, 1.89 mmol) and dry DMA (20 mL). The mixture was stirred at room temperature until the color of iodine disappeared. Ethyl 9-bromononanoate was added, and the mixture was stirred at 80° C. for 4 hours and then at room temperature overnight. (Completion of the zinc insertion reaction was checked by GCMS analysis of the hydrolyzed reaction mixture.) The reaction mixture was used without further treatment in the subsequent step. GCMS m/z 186 [M]+(ethyl nonanoate).

### Preparation of Intermediate 3

### ethyl 9-(2,6,6-trimethylcyclohex-1-en-1-yl)nonanoate

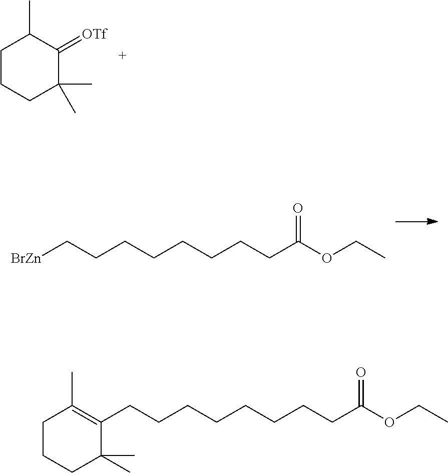

To freshly prepared ethyl 9-(bromozincio)nonanoate (37.7 mmol) in dimethylacetamide under nitrogen in a reaction tube was added 2,6,6-trimethylcyclohex-1-en-1-yl trifluoromethanesulfonate (10.8 g, 39.6 mmol) followed by tetrakis(triphenylphosphine)palladium(0) (872 mg, 0.754 mmol). The tube was sealed and the mixture was stirred at 95° C. for 2 h. The reaction mixture was allowed to cool and was then poured into diethyl ether (100 mL). The upper layer was decanted and the lower layer was washed twice with diethyl ether (2×25 mL). The combined ether layers were washed with sat NH₄Cl and brine, dried (MgSO₄) and concentrated to give crude material (˜12 g). The material was purified by flash chromatography (silica, 0 to 1.5% EtOAc in hexane). The obtained oil was stirred under vacuum for 8 h in order to remove most of the side-product, ethyl nonanoate, and was then purified by a second flash chromatography (silica, 0 to 15% toluene in hexane). The fractions were analyzed by LCMS and GCMS. The purest fractions were collected and concentrated at a temperature below 25° C. to give ethyl 9-(2,6,6-trimethylcyclohex-1-en-1-yl)nonanoate as a colorless oil (6.16 g, 53% yield over two steps). LCMS ESI+ m/z 309 [M+H]+; GCMS m/z 308 [M]+.

### Preparation of Intermediate 4

### 9-(2,6,6-trimethylcyclohex-1-en-1-yl)nonanoic acid

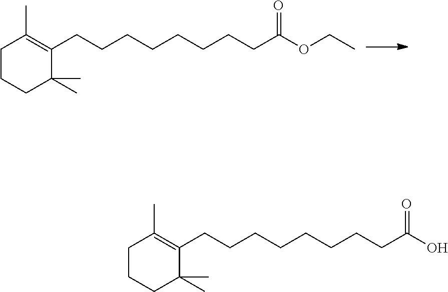 [SMILES: CCOC(=O)CCCCCCCCC1=C(C)CCCC1(C)C ; CC1=C(CCCCCCCCC(=O)O)C(C)(C)CCC1]

To ethyl 9-(2,6,6-trimethylcyclohex-1-en-1-yl)nonanoate (13.2 g, 42.9 mmol) in ethanol (80 mL) was added 4 M KOH (43 mL). The mixture was stirred at room temperature for 1.5 h. Water (350 mL) was added and the solution was washed with tert-butyl methyl ether (2×100 mL). The SimVA, aqueous phase was cooled, acidified with 4 M HCl (˜45 mL) and extracted with pentane (3×100 mL). The combined pentane extracts were washed with water (200 mL), dried (MgSO4), filtered, concentrated and dried under high vacuum. The material was redissolved in pentane (100 mL), concentrated and dried under high vacuum one more time to give 9-(2,6,6-trimethylcyclohex-1-en-1-yl)nonanoic acid as a colorless oil (11.1 g, 92% yield). MS ESI− m/z 279 [M−H]−.

### Preparation of simdiVA

### N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide

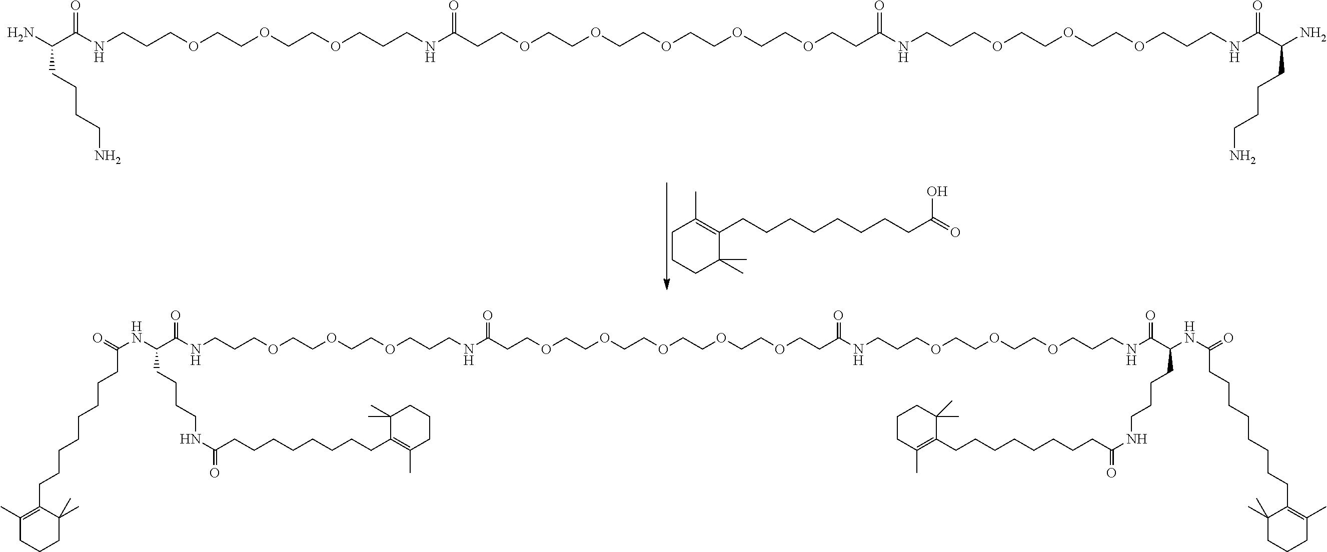

simDIVA was prepared in similar fashion as diVA from previously described N1,N19-bis((S)-16,20-diamino-15-oxo-4,7,10-trioxa-14-azaicosyl)-4,7,10,13,16-pentaoxanonadecane-1,19-diamide with the substitution of 9-(2,6,6-trimethylcyclohex-1-en-1-yl)nonanoic acid for all-trans retinoic acid. QTOF MS ESI+: m/z 2050 (M+H+)

### Example 28

### Synthesis of DiVA-PEG18

### Preparation of (2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide) (DIVA-PEG18)

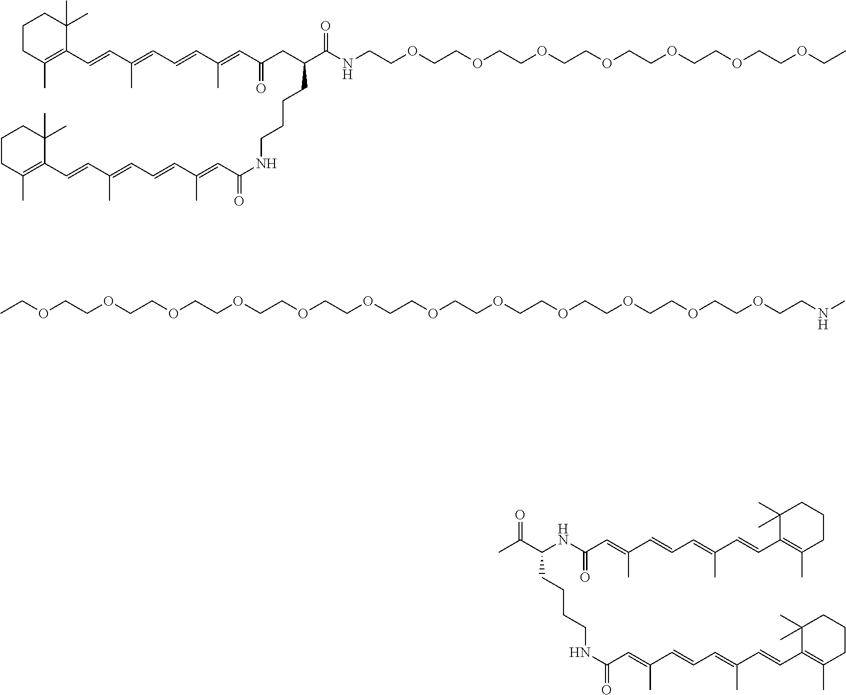

(2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide), also known as DIVA-PEG18 was prepared in similar fashion as diVA with the substitution of PEG₁₈ diamine for diamido-dPEG₁₁-diamine. LCMS ESI+: m/z 2305 (M+Na).

### Example 29

### Synthesis of TriVA

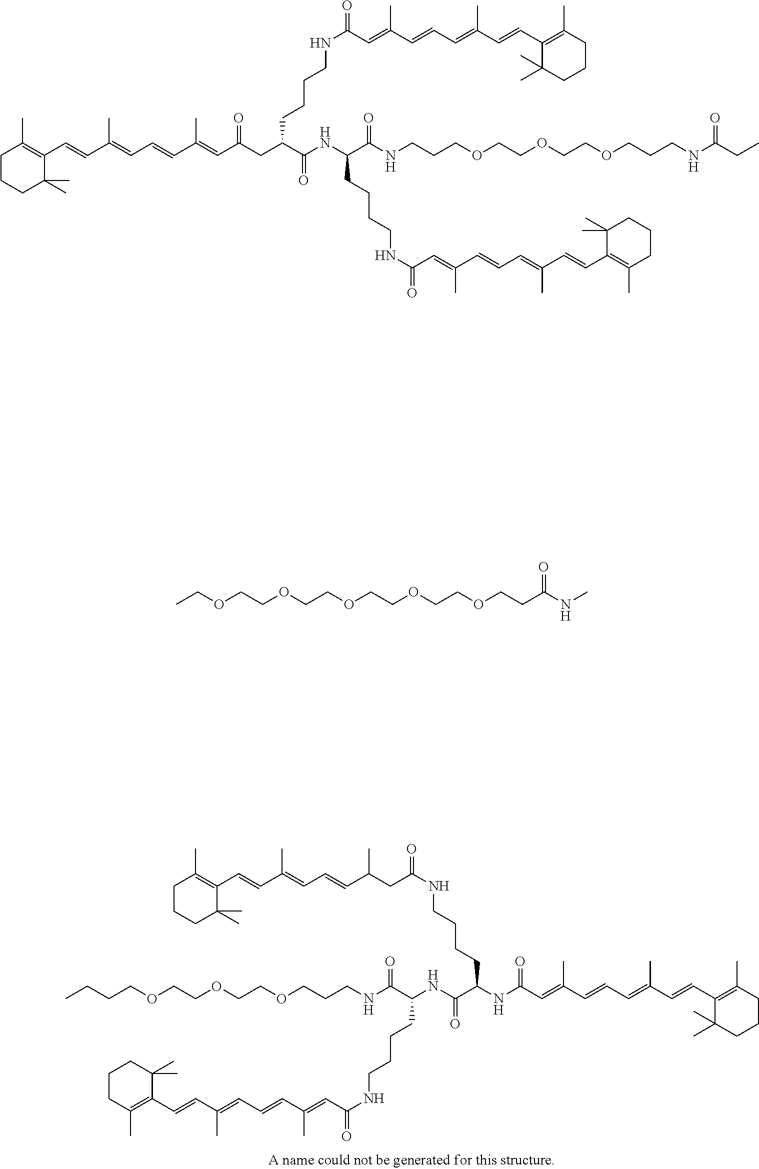

### Preparation of Intermediate 1

### (S)-methyl 6-(((benzyloxy)carbonyl)amino)-2-((S)-2,6-bis(((benzyloxy) carbonyl)amino)hexanamido) hexanoate

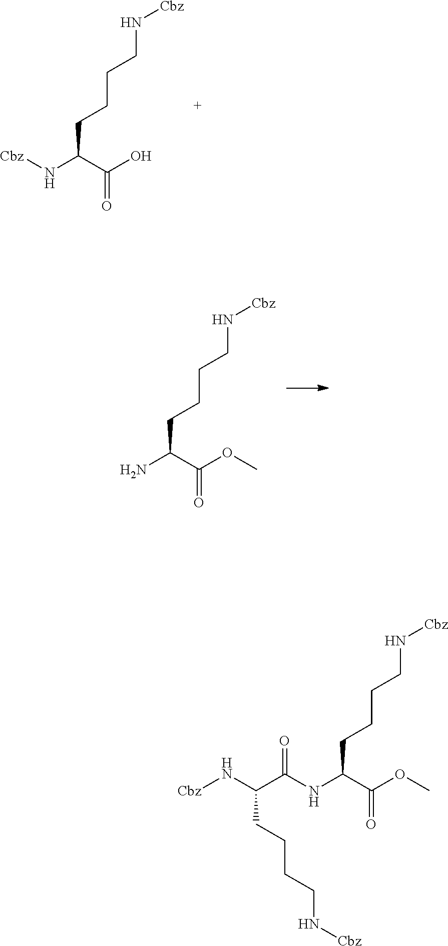 [SMILES: O=C(NCCCC[C@H](NC(=O)OCc1ccccc1)C(=O)O)OCc1ccccc1 ; COC(=O)[C@@H](N)CCCCNC(=O)OCc1ccccc1 ; COC(=O)[C@H](CCCCNC(=O)OCc1ccccc1)NC(=O)[C@H](CCCCNC(=O)OCc1ccccc1)NC(=O)OCc1ccccc1]

A flask was purged with inert gas and H-Lys(Z)—OMe HCl salt (4 g, 12.1 mmol), HOBt hydrate (1.84 g, 13.6 mmol), Z-Lys(Z)—OH (5.64 g, 13.6 mmol) are suspended in dichloromethane (50 mL). NMM (1.5 mL, 13.6 mmol) was added to the suspension and the solution became clear. A suspension EDC HCl salt (4.01 g, 20.9 mmol) and NMM (2.0 mL, 18.2 mmol) in dichloromethane (50 mL) was added over a period of 10 minutes. The reaction was stirred overnight at room temperature, then washed with 1M HCl (100 mL), H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by silica gel chromatography with a dichloromethane/methanol gradient to yield (S)-methyl 6-(((benzyloxy)carbonyl)amino)-2-((S)-2,6-bis(((benzyloxy)carbonyl)amino)hexanamido) hexanoate (6.91 g).

### Preparation of Intermediate 2

### (S)-6-(((benzyloxy)carbonyl)amino)-2-((S)-2,6-bis(((benzyloxy)carbonyl)amino)hexanamido)hexanoic acid

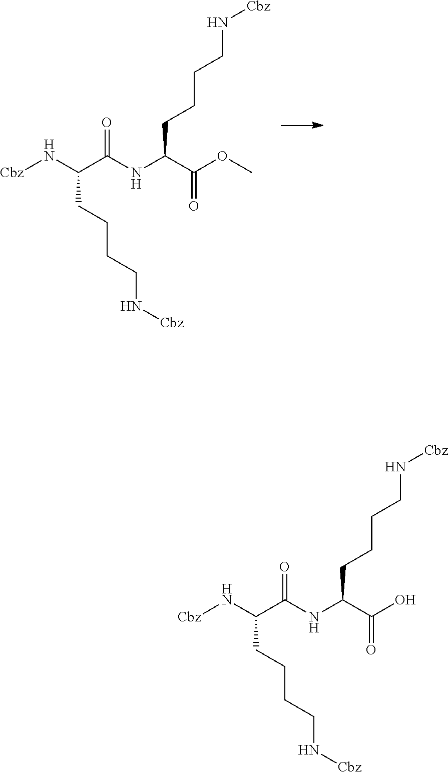 [SMILES: COC(=O)[C@H](CCCCNC(=O)OCc1ccccc1)NC(=O)[C@H](CCCCNC(=O)OCc1ccccc1)NC(=O)OCc1ccccc1 ; O=C(NCCCC[C@H](NC(=O)[C@H](CCCCNC(=O)OCc1ccccc1)NC(=O)OCc1ccccc1)C(=O)O)OCc1ccccc1]

6-(((benzyloxy)carbonyl)amino)-2-((S)-2,6-bis(((benzyloxy)carbonyl)-amino)hexanamido) hexanoate (6.91 g, 10 mmol) was dissolved with methanol (50 mL). Added KOH (2.24 g, 40 mmol) and allowed mixture to stir at 35° C. After 2 hours, quenched reaction by adding H₂O (200 mL) and washed mixture with diethyl ether (50 mL). Afterwards, adjusted the pH to ˜2 with 1M HCl acid. Extracted product with dichloromethane (3×100 mL), dried with Na₂SO₄, filtered and concentrated to yield (S)-6-(((benzyloxy)carbonyl)amino)-2-((S)-2,6-bis(((benzyloxy)carbonyl)amino)hexanamido)-hexanoic acid (4 g).

### Preparation of Intermediate 3

### (Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide

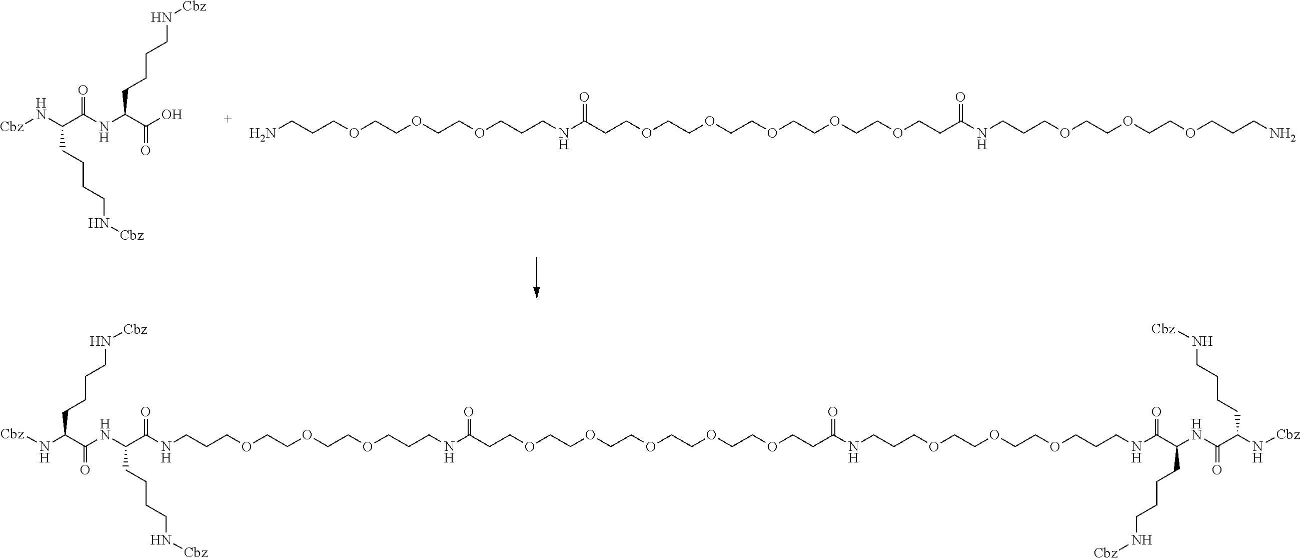 [SMILES: O=C(NCCCC[C@H](NC(=O)[C@H](CCCCNC(=O)OCc1ccccc1)NC(=O)OCc1ccccc1)C(=O)O)OCc1ccccc1 ; NCCCOCCOCCOCCCNC(=O)CCOCCOCCOCCOCCOCCC(=O)NCCCOCCOCCOCCCN ; O=C(CCOCCOCCOCCOCCOCCC(=O)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1]

A round bottom flask was purged with inert gas and diamido-dPEG₁₁-diamine (1 g, 1.35 mmol), (S)-6-(((benzyloxy)carbonyl)amino)-2-(S)-2,6-bis(((benzyloxy)carbonyl)-amino)hexanamido)hexanoic acid (2.05 g, 3.03 mmol), HOBt hydrate (409 mg, 3.03 mmol) are suspended in dichloromethane (25 mL). NMM (333 uL, 3.03 mmol) was added to the suspension and the solution became clear. A suspension EDC HCl salt (893 mg, 4.66 mmol) and NMM (445 uL, 4.05 mmol) in dichloromethane (25 mL) was added over a period of 10 minutes. The reaction was allowed to stir overnight at room temperature, then washed with 1M HCl (100 mL), H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by silica gel chromatography with a dichloromethane/methanol gradient to yield (Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (480 mg).

### Preparation of Intermediate 4

### N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide

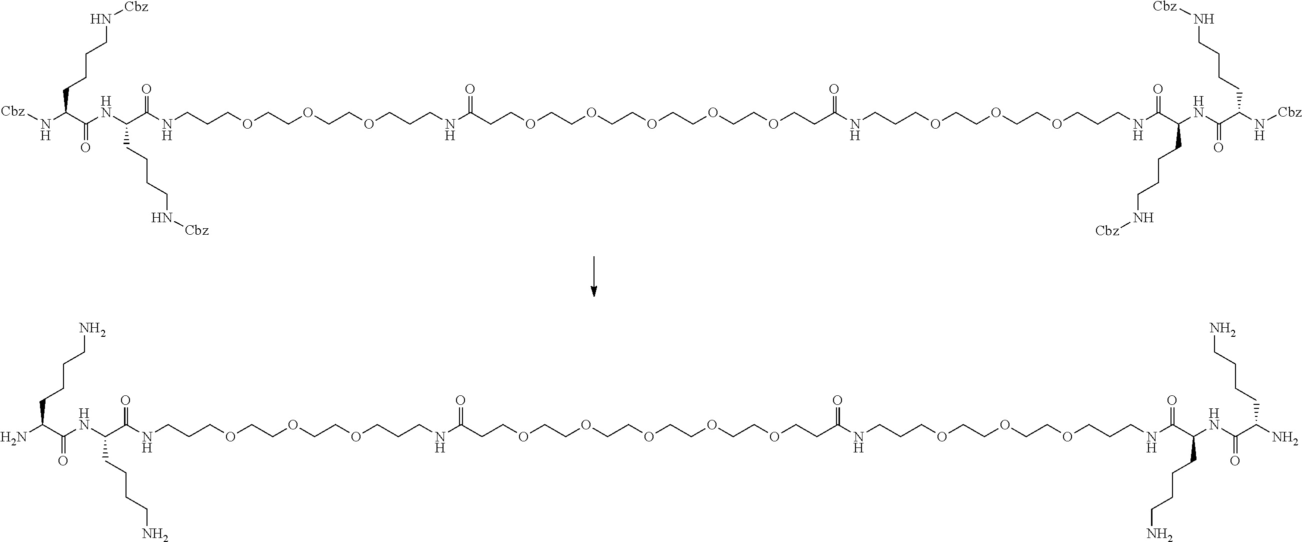 [SMILES: O=C(CCOCCOCCOCCOCCOCCC(=O)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1 ; NCCCC[C@H](NC(=O)[C@@H](N)CCCCN)C(=O)NCCCOCCOCCOCCCNC(=O)CCOCCOCCOCCOCCOCCC(=O)NCCCOCCOCCOCCCNC(=O)[C@H](CCCCN)NC(=O)[C@@H](N)CCCCN]

(Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was dissolved in methanol (30 mL) in a round bottom flask and flushed with an inert gas. 10% Pd/C (135 mg) was added and the flask was once again flushed with inert gas and then all air was removed via vacuum pump. An 8″ H₂ balloon was added and the reaction was allowed to stir at room temperature. After 2 hours, the Pd/C was removed by filtering through a pad of celite washing with methanol, and concentrated to yield N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (823 mg).

### Preparation of TriVA

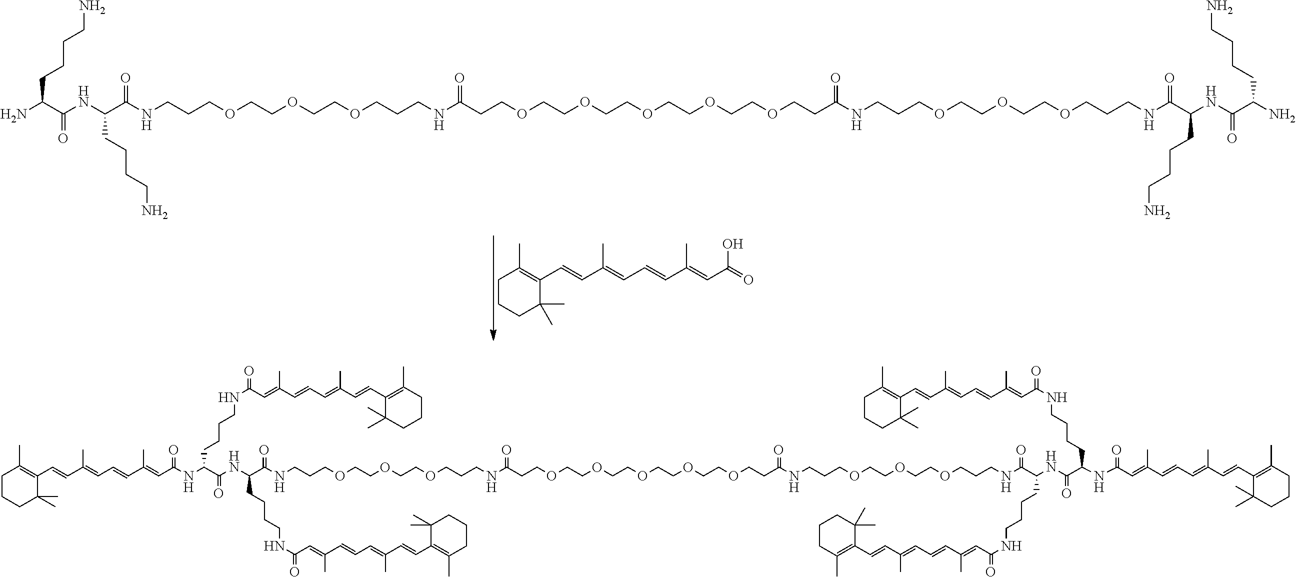

N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was stirred in dichloromethane and DMAP and retinoic acid was added. NMM was added and the solution was stirred in an aluminum foil covered round bottom flask flushed with inert gas at room temperature. A suspension of EDC HCl salt & NMM in dichloromethane (20 mL) was slowly added to reaction over a period of 10 minutes. Reaction was allowed to stir overnight at room temperature. Next day, diluted with dichloromethane to 100 mL. Washed with H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by basic alumina chromatography eluating with dichloromethane/ethanol gradient to yield TriVA (780 mg). LCMS ESI+: m/z 2972 (M+Na).

### Example 30

### Synthesis of 4TTNPB

### Preparation of N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide (4TTNPB)

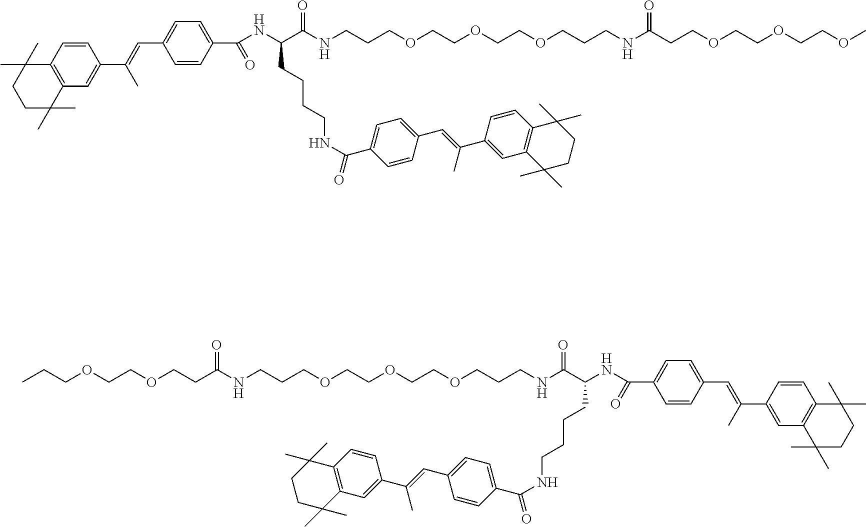

N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((4E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide, also known as 4TTNPB, was prepared in similar fashion as N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as diVA, from N1,N19-bis((S)-16,20-diamino-15-oxo-4,7,10-trioxa-14-azaicosyl)-4,7,10,13,16-pentaoxanonadecane-1,19-diamide with the substitution of TTNPB for all-trans retinoic acid. LCMS ESI+: m/z 2343 (M+Na).

### Example 31

### Synthesis of 4Myr

### Preparation of N1,N19-bis((R)-15,22-dioxo-16-tetradecanamido-4,7,10-trioxa-14,21-diazapenta-triacontyl)-4,7,10,13,16-pentaoxanonadecane-1,19-diamide (4Myr)

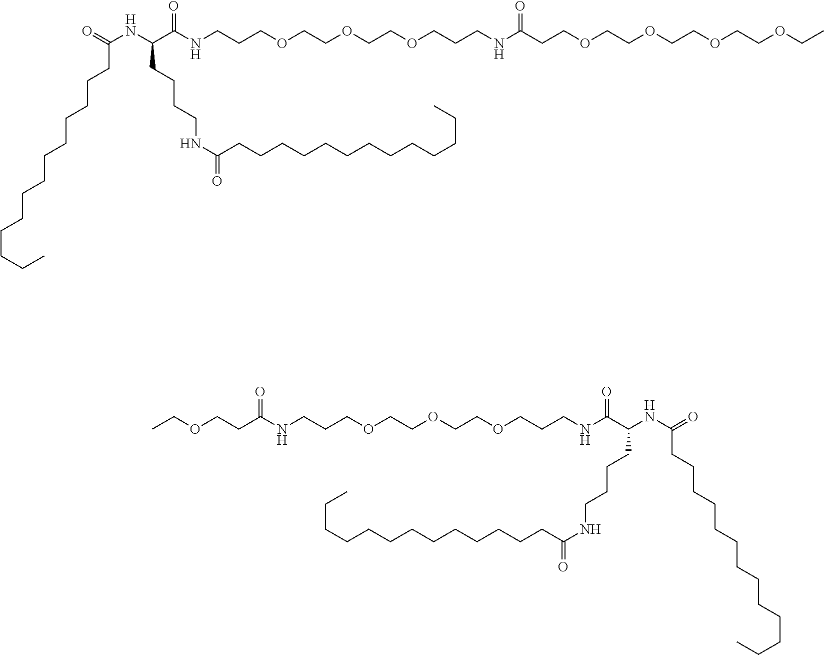

### Preparation of 4Myr

### N1,N19-bis((R)-15,22-dioxo-16-tetradecanamido-4,7,10-trioxa-14,21-diaza-penta-triacontyl)-4,7,10,13,16-pentaoxanonadecane-1,19-diamide

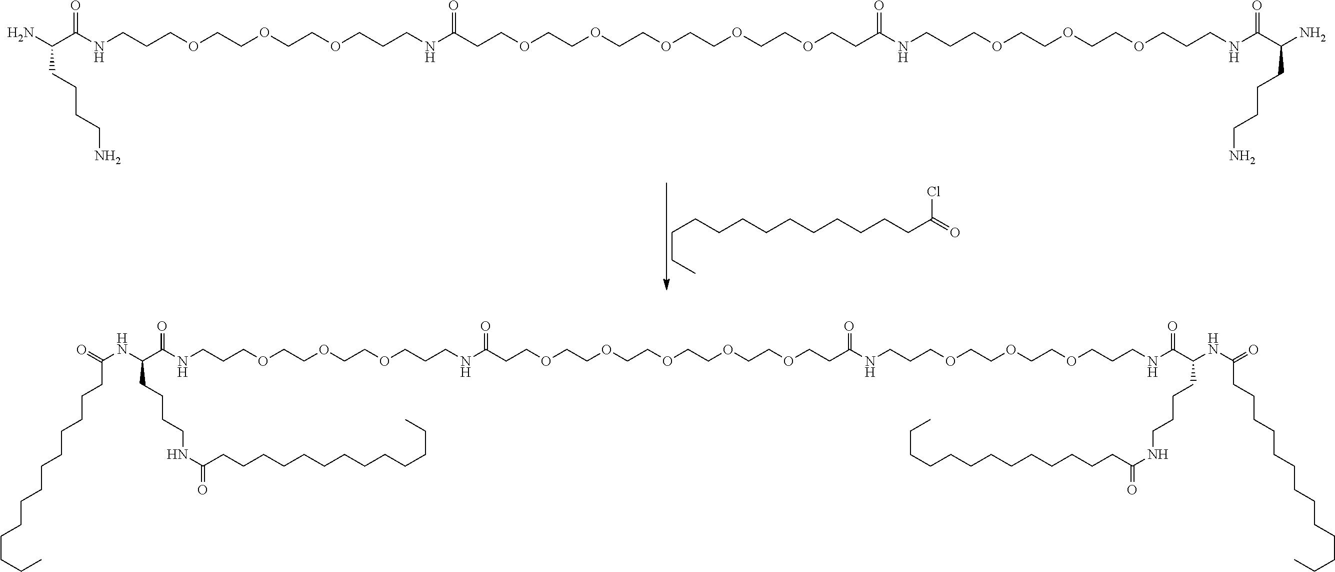 [SMILES: NCCCC[C@H](N)C(=O)NCCCOCCOCCOCCCNC(=O)CCOCCOCCOCCOCCOCCC(=O)NCCCOCCOCCOCCCNC(=O)[C@@H](N)CCCCN ; CCCCCCCCCCCCCC(=O)Cl ; CCCCCCCCCCCCCC(=O)NCCCC[C@@H](NC(=O)CCCCCCCCCCCCC)C(=O)NCCCOCCOCCOCCCNC(=O)CCOCCOCCOCCOCCOCCC(=O)NCCCOCCOCCOCCCNC(=O)[C@@H](CCCCNC(=O)CCCCCCCCCCCCC)NC(=O)CCCCCCCCCCCCC]

N1,N19-bis((S)-16,20-diamino-15-oxo-4,7,10-trioxa-14-azaicosyl)-4,7,10,13,16-pentaoxanonadecane-1,19-diamide (synthesis previously described) was dissolved in dichloromethane and placed in an ice-bath. Myristoyl chloride was added followed by triethylamine. The ice-bath was removed and the reaction was allowed to stir overnight at room temperature under a blanket of inert gas. Next day, diluted with dichloromethane to 100 mL and washed with 1M HCl (75 mL), H₂O (75 mL), saturated bicarbonate solution (75 mL) and saturated brine solution (75 mL). Back extracted all aqueous washes with dichloromethane (25 mL). Dried organics with MgSO₄, filtered and concentrated. Purification by silica gel chromatography with a dichloromethane/methanol gradient yielded N1,N19-bis((R)-15,22-dioxo-16-tetradecanamido-4,7,10-trioxa-14,21-diaza-penta-triacontyl)-4,7,10,13,16-pentaoxanonadecane-1,19-diamide (410 mg). LCMS ESI+: m/z 1841 (M+H).

### Example 32

### Synthesis of DiVA-242

### Preparation of N1,N16-bis((R,18E,20E,22E,24E)-11-((2E,4E,6E,8E)-3,7-dimethyl-9-(2,6,6-trimethyl-cyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide, also known as DIVA-242

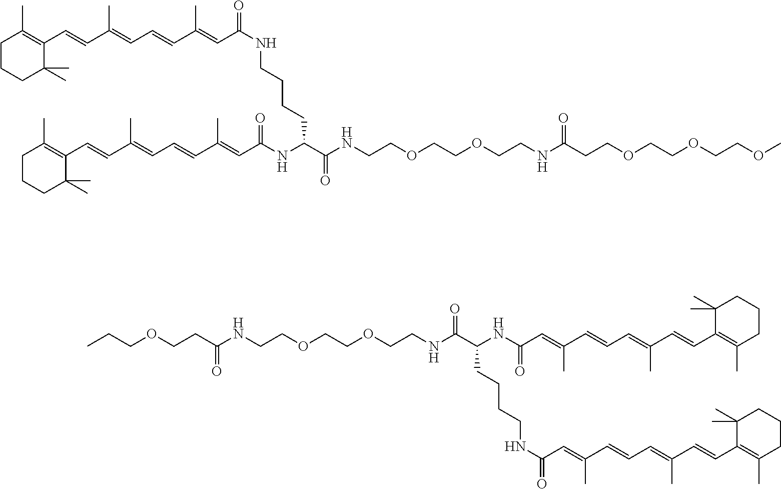

### Preparation of Intermediate 1

### di-tert-butyl (10,25-dioxo-3,6,13,16,19,22,29,32-octaoxa-9,26-diazatetratriacontane-1,34-diyl)dicarbamate

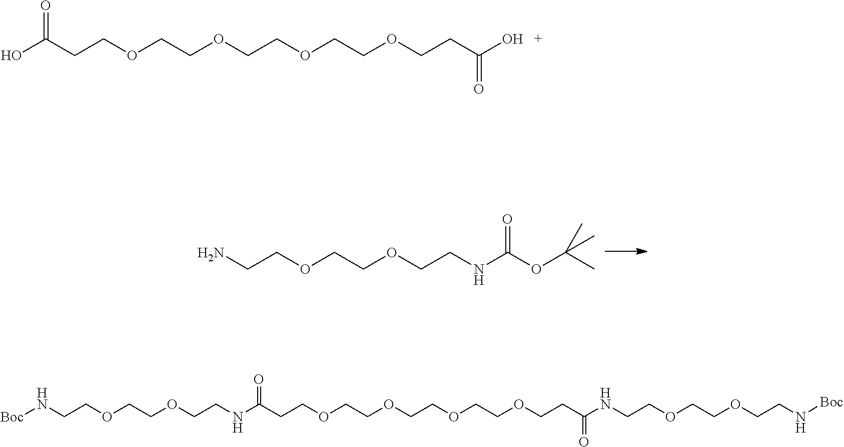

A round bottom flask containing dichloromethane (25 mL) was purged with inert gas and Bis-dPeg₄ acid (1000 mg, 3.40 mmol), N-Boc-3,6-dioxa-1,8-octane diamine (1816 uL, 7.65 mmol) and HOBt hydrate (1034 mg, 7.65 mmol) were added. NMM (841 uL, 7.65 mmol) was added to the suspension and the solution became clear. A suspension of EDC HCl salt (2249 mg, 11.7 mmol) & NMM (1121 uL, 10.2 mmol) in dichloromethane (25 mL) was added followed by DMAP (62 mg, 0.51 mmol). The reaction was allowed to stir overnight at room temperature. It was then diluted with dichloromethane to 100 mL and washed with H₂O (100 mL), 10% K₂CO₃ (100 mL) and saturated brine solution (100 mL), back extracted all aqueous washes with dichloromethane (30 mL), dried with MgSO₄, filtered and concentrated. Purification by silica gel chromatography with a dichloromethane/methanol gradient yielded di-tert-butyl (10,25-dioxo-3,6,13,16,19,22,29,32-octaoxa-9,26-diazatetratriacontane-1,34-diyl)dicarbamate (2.57 g).

### Preparation of intermediate 2

### N1,N16-bis(2-(2-(2-aminoethoxy)ethoxy)ethyl)-4,7,10,13-tetraoxahexa-decane-1,16-diamide TFA salt

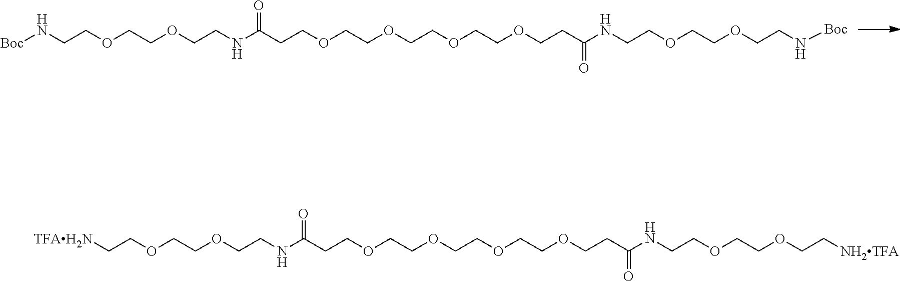

Di-tert-butyl (10,25-dioxo-3,6,13,16,19,22,29,32-octaoxa-9,26-diazatetratriacontane-1,34-diyl)dicarbamate was dissolved in dichloromethane (15 mL) and placed into an ice bath, The round bottom flask was flushed with inert gas and TFA (15 mL) was added. Mixture was allowed to stir for 20 minutes. Afterwards, the reaction mixture was concentrated to yield N1,N16-bis(2-(2-(2-aminoethoxy)ethoxy)ethyl)-4,7,10,13-tetraoxahexadecane-1,16-diamide TFA salt (1885 mg).

### Preparation of DIVA-242

### N1,N16-bis((R,18E,20E,22E,24E)-11-((2E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide

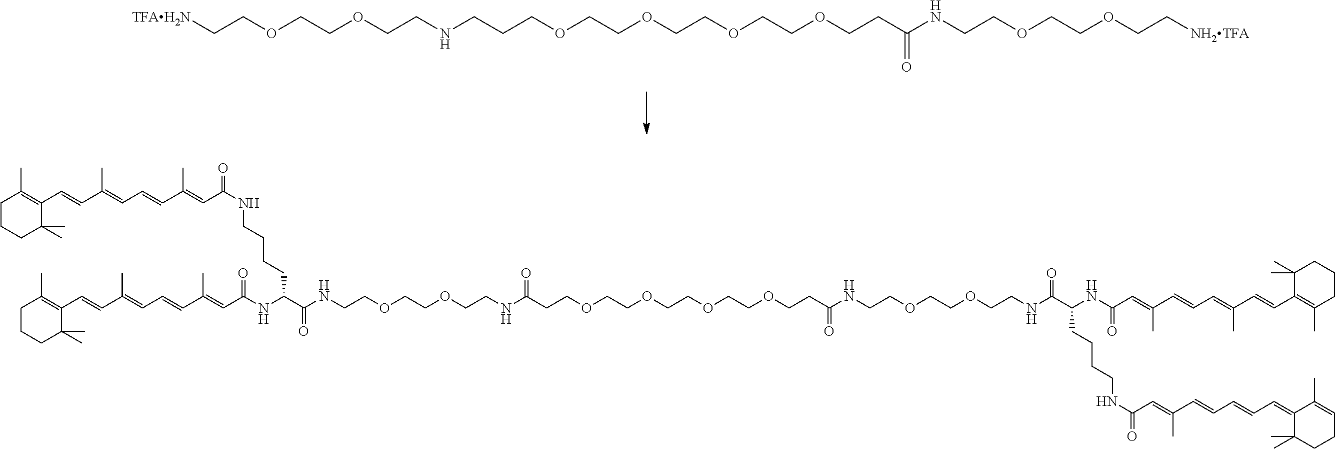

Synthesis of N1,N16-bis((R,18E,20E,22E,24E)-11-((2E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide (DIVA-242) follows the same protocol as diVA from N1,N16-bis(2-(2-(2-aminoethoxy)ethoxy)ethyl)-4,7,10,13-tetraoxahexadecane-1,16-diamide TFA salt. LCMS ESI+: m/z 1940 (M+H).

### Example 33

### In Vitro Efficacy of Fat-Soluble Vitamin Targeting Conjugate

Liposome formulations with 50 nM siRNA were tested. The liposomes were either: HEDC:S104:DOPE:Chol:PEG-DMPE:DiVA (+DiVA) or controls lacking vitamin A moieties (-DiVA) and were incubated in 96-well culture plates containing rat hepatic stellate cells for 30 minutes. After 30 minutes, medium was replaced with fresh growth medium. Forty eight hours later, cells were lysed and gp46 and GAPDH mRNA levels measured by quantitative RT-PCR (TaqMan®) assay, and gp46 levels were normalized to GAPDH levels.

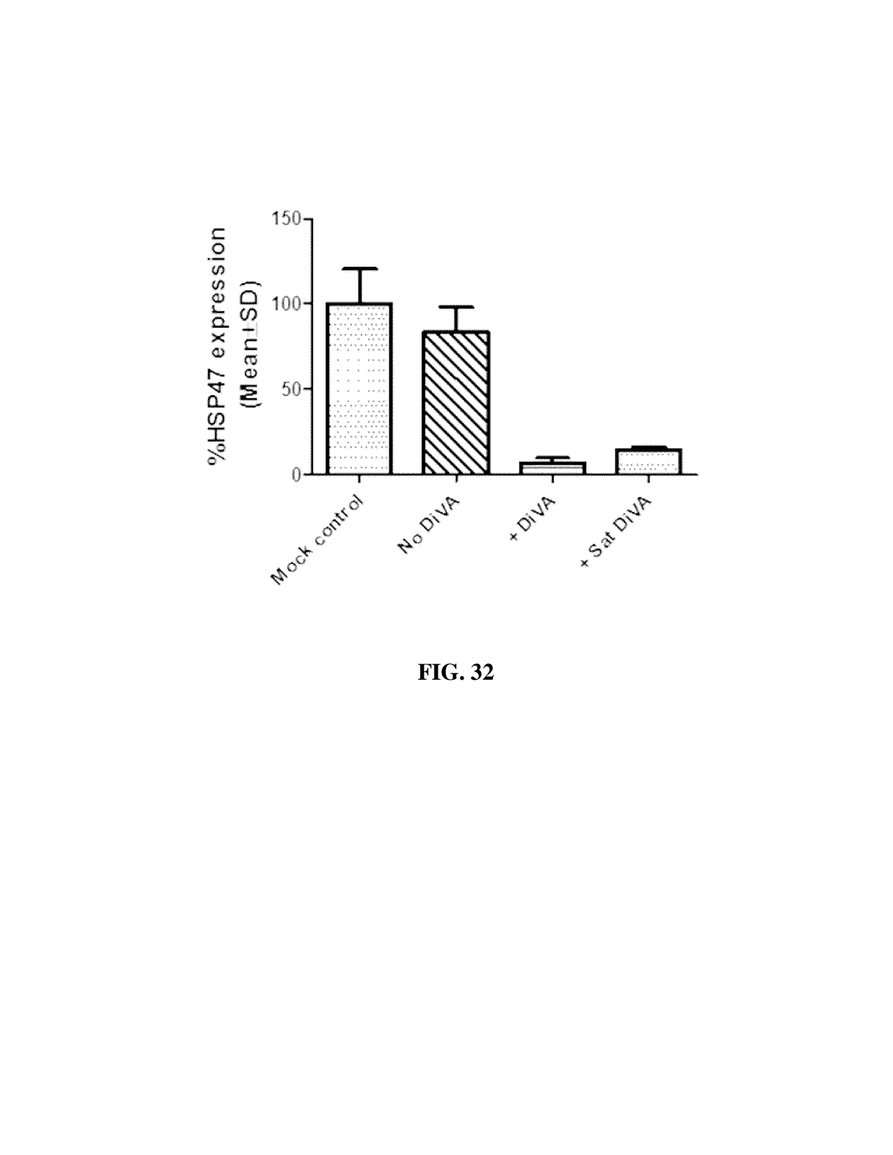

As shown in FIG. 32, in vitro efficacy (pHSC), effect of 2% DiVA siRNA was efficacious with 2% diVA and had an EC₅₀ of 14 nM. This figure shows PHSCs in 96-well plate were incubated with formulation that lacked vitamin A moieties for targeting (-DiVA), or formulation that included vitamin A moieties (+DiVA) at 50 nM siRNA. After 30 minutes, medium was replaced with fresh growth medium. Forty eight hours later, cells were lysed and gp46 and GAPDH mRNA levels measured by quantitative RT-PCR (TaqMan®) assay, and gp46 levels were normalized to GAPDH levels. Normalized gp46 levels were expressed as percent of mock control cells. Error bars indicate standard deviations (n=3). The mean gp46 level following DiVA containing treatment is significantly different from the mock control treatment (P<0.001) based on one-tailed t-test.

Comparison of DiVA AND satDiVA

Liposome formulations were transfected into rat pHSCs for 30 min in 96-well plates. After 48 hours, the cells were processed using Cells-to-C®t lysis reagents (Applied Biosystems) and HSP47 mRNA levels were quantified by qRT-PCR. HSP47 expression was normalized mock control. EC₅₀ was determined by measuring HSP47 knockdown (KD) at six half-log doses of siRNA and fitting the data to the “Classic sigmoidal dose response function” in Graphpad Prism® 5.04.

Results show that both DiVA and Sat DiVA increased KD efficacy (Table below, and FIG. 8). The EC₅₀ is 12 nM for DiVA and the EC₅₀ is 14 nM for Sat DiVA.

| Retinoid | | in vitro (pHSC) | in vivo (rat DMNQ) |
|---|---|---|---|
| Conjugate | Formulation | EC₅₀ or % KD | % KD |
| DiVA | 20:20 HEDC:S104 | EC₅₀ = 12 nM | 60% @ 0.75 mpk |
| | with 2% DiVA | | |
| satDiVA | 20:20 HEDC:S104 | EC₅₀ = 14 nM | 74% @ 0.75 mpk |
| | with 2% satDiVA | | |

### Retinoid Conjugate Vs Non-Retinoid Conjugate

Retinoid conjugates were found to be consistently more potent in vitro relative to the non-retinoid equivalents (see 4TTNBB and 4Myr vs. the retinoid conjugate equivalents satDiVA and DiVA).

| Compound | | in vitro (pHSC) |
|---|---|---|
| (Type of Conjugate) | Formulation | EC₅₀ or % KD |
| DiVA (retinoid) | 20:20 HEDC:S104 with 2% | 74% @ 50 nM |
| | DiVA | |
| satDiVA (retinoid) | 20:20 HEDC:S104 with 2% | 73% @ 50 nM |
| | satDiVA | |
| 4TTNPB (non-retinoid) | 20:20 HEDC:S104 with 2% | 34% @ 50 nM |
| | 4TTNPB | |
| 4Myr (non-retinoid) | 20:20 HEDC:S104 with 2% 4Myr | 27% @ 50 nM |

### Example 34

### In Vivo Efficacy of Fat-Soluble Vitamin Targeting Conjugate HEDC:S104:DOPE:Chol:PEG-DMPE:diva

In vivo activity of target formulation was evaluated in the short-term liver damage model (referred to as the Quick Model, DMNQ). In this model, short-term liver damage is induced by treatment with a hepatotoxic agent such as dimethylnitrosamine (DMN), and is accompanied by the elevation of gp46 mRNA levels. To induce these changes, male Sprague-Dawley rats were injected intraperitoneally with DMN on six consecutive days. At the end of the DMN treatment period, the animals were randomized to groups based upon individual animal body weight. Formulations were administered as a single IV dose, and given one hour after the last injection of DMN. Twenty four hours later, liver lobes were excised and both gp46 and MRPL19 mRNA levels were determined by quantitative RT-PCR (TaqMan®) assay. mRNA levels for gp46 were normalized to MRPL19 levels.

The results (FIG. 9) show a correlation between the amount of retinoid conjugate and efficacy is evident. Only 0.25 mol % is required to see a significant effect in the rat DMNQ model. With 2 mol % DiVA a robust knockdown of gp46 expression is observed. FIG. 9 shows male Sprague-Dawley rats that were treated with DMN at 10 mg/kg on day 1, 2, 3 and 5 mg/kg on day 4, 5, 6 through intraperitoneal dosing to induce liver damage. Animals (n=8/group) were injected intravenously either with formulations containing 0, 0.25, 0.5, 1, and 2% DiVA at a dose of 0.75 mg/kg siRNA, or PBS (vehicle), one hour after the last injection of DMN. Twenty four hours later, total siRNA was purified from a section of the right liver lobe from each animal and stored at 4° C. until RNA isolation. Control groups included a PBS vehicle group (DMN-treated) and naïve (untreated; no DMN) group. After subtracting background gp46 mRNA levels determined from the naïve group, all test group values were normalized to the average gp46 mRNA of the vehicle group (expressed as a percent of the vehicle group).

Male Sprague Dawley rats (130-160 g) were treated DMN through intraperitoneal dosing to induce liver fibrosis. The DMN treatment regimen was 3 times each week (Mon, Wed, and Fri) with 10 mg/kg (i.e., 5.0 mg/mL of DMN at a dose of 2.0 mL/kg body weight) for first 3 weeks and half dose of 5 mg/kg (i.e., 5 mg/mL of DMN at a dose of 1.0 mL/kg) from day 22 to 57. The sham group animals were injected with PBS (solvent for DMN) using the same schedule. On Day 22, 24 h post the last DMN treatment, blood samples were collected and assayed for liver disease biomarkers to confirm the effectiveness of the DMN treatment. DMN treated animals were assigned to different treatment groups based on body weight and ensure that the mean body weights and the range of body weights of the animals in each group have no significant difference. Animals from pretreatment group were sacrificed on day 25 to evaluate the disease progression stage prior to treatment begins. Treatments with formulations containing gp46 siRNA were started at day 25 with 2 treatments/week at specified siRNA dose for a total of 10 times. On day 59, 48 hours after last formulation treatment and 72 hours after last DMN treatment, animals were sacrificed by CO₂ inhalation. Liver lobes were excised and both gp46 and MRPL19 mRNA levels were determined by quantitative RT-PCR (TaqMan) assay. mRNA levels for gp46 were normalized to MRPL19 levels.

What is claimed is:

1. A stellate cell-specific drug carrier comprising a stellate cell-specific amount of a retinoid derivative and/or a vitamin A analogue, and a drug carrier component other than the retinoid derivative and/or a vitamin A analogue.

2. The drug carrier according to claim 1, wherein the retinoid derivative comprises a compound consisting of the structure (retinoid)ₘ-linker-(retinoid)ₙ, wherein m and n are independently 0, 1, 2, or 3, except that m and n are not both zero; and wherein the linker comprises a polyethylene glycol (PEG) or PEG-like molecule.

3. The drug carrier according to claim 2, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

4. The drug carrier according to claim 2, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

5. The drug carrier according to claim 2, wherein the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

6. The drug carrier according to claim 5, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

7. The drug carrier according to claim 6, wherein the compound is of formula 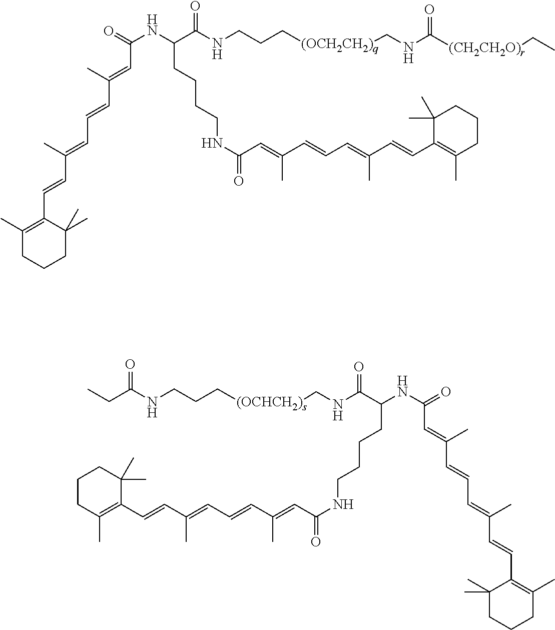
wherein q, r, and s are each independently 1, 2, 3, 4, 5, 6, 7, 8, 9, or 10.

8. The drug carrier according to claim 6, wherein the compound is of formula

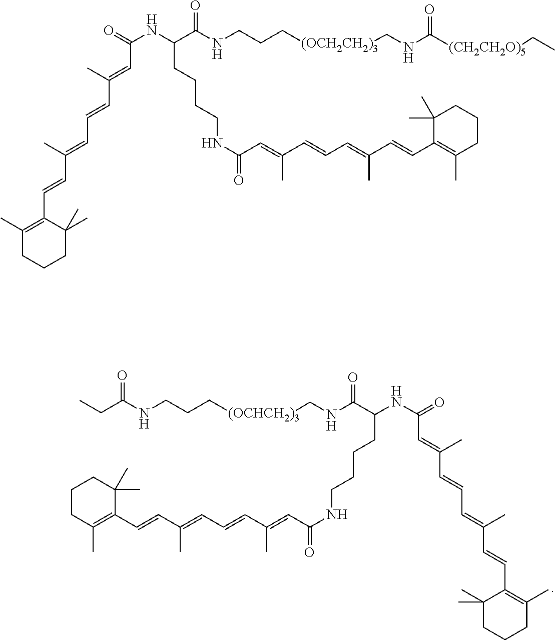

9. The drug carrier according to claim 2, wherein the PEG is monodisperse.

10. The drug carrier according to claim 1, wherein the retinoid derivative comprises a compound consisting of the structure (lipid)ₘ-linker-(retinoid)ₙ, wherein m and n are independently 0, 1, 2, or 3, except that m and n are not both zero; and wherein the linker comprises a polyethylene glycol (PEG) molecule.

11. The drug carrier according to claim 10, wherein the lipid is selected from one or more of the group consisting of DODC, HEDODC, DSPE, DOPE, and DC-6-14.

12. The drug carrier according to claim 10, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

13. The drug carrier according to claim 10, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

14. The drug carrier according to claim 13, wherein the retinoid derivative is selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

15. The drug carrier according to claim 1, further comprising a liposomal composition.

16. The drug carrier according to claim 15, wherein the liposomal composition comprises a lipid vesicle comprising a bilayer of lipid molecules.

17. The drug carrier according to claim 16, wherein the lipid molecules comprise one or more lipids selected from the group consisting of HEDC, DODC, HEDODC, DSPE, DOPE, and DC-6-14.

18. The drug carrier according to claim 17, wherein the lipid molecules further comprise S104.

19. A medicine comprising: (i) a stellate cell-specific drug carrier of claim 1, and (ii) a drug in an amount effective for controlling the activity or growth of stellate cells.

20. The medicine according to claim 19, wherein the drug for controlling the activity or growth of stellate cells is selected from the group consisting of an siRNA, ribozyme, antisense nucleic acid and DNA/RNA chimera polynucleotide; or a vector expressing same.


SEQUENCE LISTING (7 sequences)

SEQ ID NO: 1 - DNA, 21 bp, Artificial Sequence
  Synthetic oligonucleotide
ggacaggccucuacaacuatt

SEQ ID NO: 2 - DNA, 21 bp, Artificial Sequence
  Synthetic oligonucleotide
uaguuguagaggccugucctt

SEQ ID NO: 3 - DNA, 21 bp, Artificial Sequence
  Synthetic oligonucleotide
ggacaggccuguacaacuatt

SEQ ID NO: 4 - DNA, 21 bp, Artificial Sequence
  Synthetic oligonucleotide
uaguuguacaggccugucctt

SEQ ID NO: 5 - RNA, 25 nt, Artificial sequence
  Synthetic oligonucleotide
guuccaccauaagaugguagacaac

SEQ ID NO: 6 - RNA, 25 nt, Artificial sequence
  Synthetic oligonucleotide
ccacaaguuuuauauccaaucuagc

SEQ ID NO: 7 - RNA, 19 nt, Artificial sequence
  Synthetic oligonucleotide
gaaaccuguagaggccgca